SEQUENCE LISTING (5 sequences)

SEQ ID NO: 1 - protein, 1049 aa, Bacillus megaterium [sequence omitted]

SEQ ID NO: 2 - DNA, 3150 bp, Bacillus megaterium
  source 1..3150: mol_type = genomic DNA
atgacaattaaagaaatgcctcagccaaaaacgtttggagagcttaaaaatttaccgttattaaacacagataaaccggttcaagctttgatgaaaattgcggatgaattaggagaaatctttaaattcgaggcgcctggtcgtgtaacgcgctacttatcaagtcagcgtctaattaaagaagcatgcgatgaatcacgctttgataaaaacttaagtcaagcgcttaaatttgtacgtgattttgcaggagacgggttatttacaagctggacgcatgaaaaaaattggaaaaaagcgcataatatcttacttccaagcttcagtcagcaggcaatgaaaggctatcatgcgatgatggtcgatatcgccgtgcagcttgttcaaaagtgggagcgtctaaatgcagatgagcatattgaagtaccggaagacatgacacgtttaacgcttgatacaattggtctttgcggctttaactatcgctttaacagcttttaccgagatcagcctcatccatttattacaagtatggtccgtgcactggatgaagcaatgaacaagctgcagcgagcaaatccagacgacccagcttatgatgaaaacaagcgccagtttcaagaagatatcaaggtgatgaacgacctagtagataaaattattgcagatcgcaaagcaagcggtgaacaaagcgatgatttattaacgcatatgctaaacggaaaagatccagaaacgggtgagccgcttgatgacgagaacattcgctatcaaattattacattcttaattgcgggacacgaaacaacaagtggtcttttatcatttgcgctgtatttcttagtgaaaaatccacatgtattacaaaaagcagcagaagaagcagcacgagttctagtagatcctgttccaagctacaaacaagtcaaacagcttaaatatgtcggcatggtcttaaacgaagcgctgcgcttatggccaactgctcctgcgttttccctatatgcaaaagaagatacggtgcttggaggagaatatcctttagaaaaaggcgacgaactaatggttctgattcctcagcttcaccgtgataaaacaatttggggagacgatgtggaagagttccgtccagagcgttttgaaaatccaagtgcgattccgcagcatgcgtttaaaccgtttggaaacggtcagcgtgcgtgtatcggtcagcagttcgctcttcatgaagcaacgctggtacttggtatgatgctaaaacactttgactttgaagatcatacaaactacgagctggatattaaagaaactttaacgttaaaacctgaaggctttgtggtaaaagcaaaatcgaaaaaaattccgcttggcggtattccttcacctagcactgaacagtctgctaaaaaagtacgcaaaaaggcagaaaacgctcataatacgccgctgcttgtgctatacggttcaaatatgggaacagctgaaggaacggcgcgtgatttagcagatattgcaatgagcaaaggatttgcaccgcaggtcgcaacgcttgattcacacgccggaaatcttccgcgcgaaggagctgtattaattgtaacggcgtcttataacggtcatccgcctgataacgcaaagcaatttgtcgactggttagaccaagcgtctgctgatgaagtaaaaggcgttcgctactccgtatttggatgcggcgataaaaactgggctactacgtatcaaaaagtgcctgcttttatcgatgaaacgcttgccgctaaaggggcagaaaacatcgctgaccgcggtgaagcagatgcaagcgacgactttgaaggcacatatgaagaatggcgtgaacatatgtggagtgacgtagcagcctactttaacctcgacattgaaaacagtgaagataataaatctactctttcacttcaatttgtcgacagcgccgcggatatgccgcttgcgaaaatgcacggtgcgttttcaacgaacgtcgtagcaagcaaagaacttcaacagccaggcagtgcacgaagcacgcgacatcttgaaattgaacttccaaaagaagcttcttatcaagaaggagatcatttaggtgttattcctcgcaactatgaaggaatagtaaaccgtgtaacagcaaggttcggcctagatgcatcacagcaaatccgtctggaagcagaagaagaaaaattagctcatttgccactcgctaaaacagtatccgtagaagagcttctgcaatacgtggagcttcaagatcctgttacgcgcacgcagcttcgcgcaatggctgctaaaacggtctgcccgccgcataaagtagagcttgaagccttgcttgaaaagcaagcctacaaagaacaagtgctggcaaaacgtttaacaatgcttgaactgcttgaaaaatacccggcgtgtgaaatgaaattcagcgaatttatcgcccttctgccaagcatacgcccgcgctattactcgatttcttcatcacctcgtgtcgatgaaaaacaagcaagcatcacggtcagcgttgtctcaggagaagcgtggagcggatatggagaatataaaggaattgcgtcgaactatcttgccgagctgcaagaaggagatacgattacgtgctttatttccacaccgcagtcagaatttacgctgccaaaagaccctgaaacgccgcttatcatggtcggaccgggaacaggcgtcgcgccgtttagaggctttgtgcaggcgcgcaaacagctaaaagaacaaggacagtcacttggagaagcacatttatacttcggctgccgttcacctcatgaagactatctgtatcaagaagagcttgaaaacgcccaaagcgaaggcatcattacgcttcataccgctttttctcgcatgccaaatcagccgaaaacatacgttcagcacgtaatggaacaagacggcaagaaattgattgaacttcttgatcaaggagcgcacttctatatttgcggagacggaagccaaatggcacctgccgttgaagcaacgcttatgaaaagctatgctgacgttcaccaagtgagtgaagcagacgctcgcttatggctgcagcagctagaagaaaaaggccgatacgcaaaagacgtgtgggctgggtaa

SEQ ID NO: 3 - protein, 1080 aa, unidentified
  source 1..1080: note = Myceliophthora thermophila
MADKTTETVPIPGPPGLPLVGNALAFDSELPLRTFQEFAEEYGEIYRLTLPTGTTLVVSSQALVHELCDDKRFKKPVAAALAEVRNGVNDGLFTAREEEPNWGIAHRILMPAFGPASIQGMFTEMHEIASQLALKWARHGPDTPIFVTDDFTRLTLDTLALCTMNFRFNSYYHDELHPFINAMGNFLTESGARAMRPAITSIFHQAANRKYWEDIEVLRKTAQGVLDTRRKHPTNRKDLLSAMLDGVDAKTGQKLSDSSIIDNLITFLIAGHETTSGLLSFAFYLLIKHQDAYRKAQEEVDRVIGKGPIKVEHIKKLPYIAAVLRETLRLCPTIPIINRAAKQDEVIGGKYAVAKDQRLALLLAQSHLDPAVYGETAKQFIPERMLDENFERLNREYPDCWKPFGTGMRACIGRPFAWQEAVLVMAMLLQNFDFVLHDPYYELHYKQTLTTKPKDFYMRAILRDGLTATELEHRLAGNAASVARSGGGGGGPSKPTAQKTSPAEAKPMSIFYGSNTGTCESLAQRLATDAASHGYAAAAVEPLDTATEKLPTDRPVVIITASFEGQPPDNAAKFCGWLKNLEGDELKNVSYAVFGCGHHDWSQTFHRIPKLVHQTMKAHGASPICDEGLTDVAEGNMFTDFEQWEDDVFWPAVRARYGAAGAVAETEDAPGSDGLNIHFSSPRSSTLRQDVREATVVGEALLTAPDAPPKKHIEVQLPDGATYKVGDYLAVLPVNSKESIGRVMRKFQLSWDSHVTIASDRWTALPTGTPVPAYDVLGSYVELSQPATKRGILRLADAAEDEATKAELQKLAGDLYTSEISLKRASVLDLLDRFPSISLPFGTFLSLLPPIRPRQYSISSSPLNDPSRATLTYSLLDSPSLANPSRRFVGVATSYLSSLVRGDKLLVSVRPTHTAFRLPDEDKMGETAIICVGAGSGLAPFRGFIQERAALLAKGTQLAAALLFYGCRSPEKDDLYRDEFDKWQESGAVDVRRAFSRVDSDDTEARGCRHVQDRLWHDREEVKALWDRGARVYVCGSRQVGEGVKTAMGRIVLGEEDAEDAISKWYETVRNDRYATDVFD

SEQ ID NO: 4 - DNA, 3243 bp, synthetic construct
  misc_feature 1..3243: note = Synthetic
  source 1..3243: mol_type = other DNA
atggcggataagaccaccgaaaccgtgccgattccgggtccgccgggcctgccgctggttggtaatgcgctggcgtttgatagcgaactgccgctgcgtaccttccaggaatttgcggaggaatacggcgagatctatcgtctgaccctgccgaccggtaccaccctggtggttagcagccaagcgctggttcacgaactgtgcgacgataagcgtttcaagaagccggttgctgcggcgctggcggaagtgcgtaacggcgttaacgacggtctgtttaccgcgcgtgaagaggagccgaactggggcatcgcgcaccgtattctgatgccggcgtttggtccggcgagcattcagggcatgtttaccgaaatgcacgagatcgcgagccaactggcgctgaaatgggcgcgtcacggtccggacaccccgattttcgttaccgacgattttacccgtctgaccctggataccctggcgctgtgcaccatgaacttccgttttaacagctactatcacgacgaactgcacccgttcatcaacgcgatgggcaactttctgaccgagagcggtgcgcgtgcgatgcgtccggcgatcaccagcattttccaccaggcggcgaaccgtaagtactgggaagatattgaggttctgcgtaaaaccgcgcaaggtgtgctggacacccgtcgtaagcacccgaccaaccgtaaagatctgctgagcgcgatgctggacggcgtggatgcgaaaaccggtcagaaactgagcgacagcagcatcattgataacctgatcacctttctgattgcgggccacgaaaccaccagcggtctgctgagcttcgcgttttacctgctgattaagcaccaggacgcgtatcgtaaagcgcaagaagaggtggatcgtgttatcggcaagggcccgattaaagttgaacacatcaagaaactgccgtacatcgcggcggtgctgcgtgaaaccctgcgtctgtgcccgaccattccgatcattaaccgtgcggcgaagcaggacgaagttatcggtggcaagtacgcggtggcgaaagatcagcgtctggcgctgctgctggcgcaaagccacctggacccggcggtttatggcgaaaccgcgaagcaattcattccggagcgtatgctggacgaaaactttgagcgtctgaaccgtgagtatccggattgctggaaaccgttcggtaccggcatgcgtgcgtgcatcggtcgtccgtttgcgtggcaggaagcggtgctggttatggcgatgctgctgcaaaacttcgactttgttctgcacgatccgtactatgagctgcactacaagcagaccctgaccaccaagccgaaagacttctatatgcgtgcgatcctgcgtgatggcctgaccgcgaccgaactggagcaccgtctggcgggtaacgcggcgagcgtggcgcgtagcggtggcggtggcggtggcccgagcaaaccgaccgcgcagaaaaccagcccggcggaagcgaaaccgatgagcatcttctacggcagcaacaccggtacctgcgagagcctggcgcaacgtctggcgaccgatgcggcgagccacggttatgctgcggcggcggtggaaccgctggacaccgcgaccgagaagctgccgaccgatcgtccggtggttatcattaccgcgagcttcgagggtcagccgccggacaacgcggcgaagttttgcggctggctgaaaaacctggaaggtgatgagctgaaaaacgtgagctacgcggttttcggttgcggccaccacgactggagccagacctttcaccgtattccgaagctggttcaccaaaccatgaaagcgcacggtgcgagcccgatctgcgacgaaggcctgaccgatgtggcggagggtaacatgttcaccgattttgaacaatgggaggacgatgtgttctggccggcggttcgtgcgcgttatggcgcggcgggtgcggttgcggaaaccgaggacgcgccgggtagcgatggtctgaacatccactttagcagcccgcgtagcagcaccctgcgtcaggacgtgcgtgaagcgaccgtggttggtgaagcgctgctgaccgcgccggatgcgccgccgaagaaacacattgaagttcaactgccggacggcgcgacctacaaagtgggtgattatctggcggtgctgccggttaacagcaaggagagcattggtcgtgttatgcgtaaattccagctgagctgggacagccacgtgaccatcgcgagcgatcgttggaccgcgctgccgaccggtaccccggtgccggcgtacgacgttctgggtagctatgtggagctgagccaaccggcgaccaaacgtggtatcctgcgtctggcggatgcggcggaagatgaggcgaccaaggcggaactgcaaaaactggcgggtgatctgtacaccagcgagattagcctgaaacgtgcgagcgttctggacctgctggatcgtttcccgagcatcagcctgccgttcggtacctttctgagcctgctgccgccgattcgtccgcgtcaatacagcatcagcagcagcccgctgaacgacccgagccgtgcgaccctgacctatagcctgctggatagcccgagcctggcgaacccgagccgtcgtttcgtgggcgttgcgaccagctacctgagcagcctggttcgtggtgacaagctgctggtgagcgttcgtccgacccacaccgcgtttcgtctgccggacgaagataaaatgggtgaaaccgcgatcatttgcgtgggtgcgggtagcggtctggcgccgttccgtggttttatccaggaacgtgcggcgctgctggcgaaaggtacccaactggcggcggcgctgctgttctacggttgccgtagcccggagaaggacgatctgtatcgtgacgaattcgataaatggcaagagagcggtgcggtggatgttcgtcgtgcgtttagccgtgttgatagcgacgataccgaggcgcgtggttgccgtcacgttcaggaccgtctgtggcacgatcgtgaagaggtgaaggcgctgtgggaccgtggcgcgcgtgtgtacgtttgcggtagccgtcaagtgggcgaaggtgttaaaaccgcgatgggccgtatcgtgctgggtgaagaggacgcggaggatgcgatcagcaagtggtatgaaaccgtgcgtaatgaccgttatgcgaccgatgtgttcgactaa

SEQ ID NO: 5 - protein, 342 aa, unidentified
  source 1..342: note = Candida antarctica
MKLLSLTGVAGVLATCVAATPLVKRLPSGSDPAFSQPKSVLDAGLTCQGASPSSVSKPILLVPGTGTTGPQSFDSNWIPLSTQLGYTPCWISPPPFMLNDTQVNTEYMVNAITALYAGSGNNKLPVLTWSQGGLVAQWGLTFFPSIRSKVDRLMAFAPDYKGTVLAGPLDALAVSAPSVWQQTTGSALTTALRNAGGLTQIVPTTNLYSATDEIVQPQVSNSPLDSSYLFNGKNVQAQAVCGPLFVIDHAGSLTSQFSYVVGRSALRSTTGQARSADYGITDCNPLPANDLTPEQKVAAAALLAPAAAAIVAGPKQNCEPDLMPYARPFAVGKRTCSGIVTP

(19) United States
(12) Patent Application Publication
(10) Pub. No.: US 2024/0287561 A1
(43) Pub. Date: Aug. 29, 2024

(54) BIOSYNTHETIC PRODUCTION OF MACROCYCLIC MUSK LACTONES FROM FATTY ACIDS

(71) Applicant: Conagen Inc., Bedford, MA (US)

(72) Inventors: Hui Chen, North Bilerica, MA (US); Manuel Garcia Venegas, West Chester, OH (US); Oliver Yu, Lexington, MA (US); Michael Nazaro, Bedford, MA (US); Andrew Stewart, Bedford, MA (US)

(73) Assignee: Conagen Inc., Bedford, MA (US)

(21) Appl. No.: 18/463,336

(22) Filed: Sep. 8, 2023

Related U.S. Application Data
(63) Continuation of application No. PCT/US22/19529, filed on Mar. 9, 2022.
(60) Provisional application No. 63/158,843, filed on Mar. 9, 2021.
(60) Provisional application No. 63/182,242, filed on Apr. 30, 2021.

(51) Int. Cl. C12P 17/08; C07D 313/00

(52) U.S. Cl. CPC C12P 17/08; C07D 313/00

(57) ABSTRACT

Provided herein are biosynthetic methods for producing lactones, such as macrocyclic lactones, from fatty acids. The lactones produced using and/or during the biosynthetic methods are also provided.

### RELATED APPLICATIONS

This application claims the benefit under 35 U.S.C. § 119(e) to U.S. Provisional Application No. 63/158,843, filed on Mar. 9, 2021 and entitled “BIOSYNTHETIC PRODUCTION OF MACROCYCLIC MUSK LACTONES FROM FATTY ACIDS” and to U.S. Provisional Application No. 63/182,242, filed on Apr. 30, 2021 and entitled “BIOSYNTHETIC PRODUCTION OF MACROCYCLIC MUSK LACTONES FROM FATTY ACIDS,” the entire contents of each of which are incorporated herein by reference.

### SEQUENCE LISTING PARAGRAPH

The instant application contains a Sequence Listing which has been submitted in ASCII format via EFS-Web and is hereby incorporated by reference in its entirety. Said ASCII copy, created on Mar. 9, 2022, is named C149770047WO00-SEQ-ZJG and is 30,286 bytes in size.

### FIELD OF THE INVENTION

The field of the invention relates to methods and processes useful in the production of lactone compounds (e.g., macrocyclic lactone compounds that may be flavor- or fragrance-bearing).

### BACKGROUND

Musk lactones are generally scarce and expensive. Currently, only a limited number of macrocyclic musk lactones are available and only with limited differentiated musky notes.

Most of the existing musk lactones are chemically synthesized, and few are of natural.

### SUMMARY

The present disclosure, in some aspects, provide methods (e.g., biosynthetic methods) of producing lactones, such as macrocyclic lactones, from fatty acids. In some embodiments, the lactones, such as macrocyclic lactones, produced using the methods described herein have differentiated musky notes. As described herein, Cytochrome P450 enzymes that have hydroxylase activity can carry out the first step of the biosynthetic method described herein, i.e., converting fatty acids to ω-1, ω-2, ω-3 hydroxyl fatty acids, or combinations thereof. The second step of the biosynthetic methods described herein, the macrolactonization of the ω-1, ω-2, ω-3 hydroxyl fatty acids, can be carried out by a lipase. Novel lactones, such as macrocyclic lactones are also provided.

Accordingly, some aspects of the present disclosure provide methods of producing a lactone, the method comprising:
(i) preparing a first reaction mixture comprising one or more fatty acids, a cytochrome P450 hydroxylase, and NADPH;
(ii) incubating the first reaction mixture of for a sufficient time to produce hydroxyl fatty acids selected from ω-1 hydroxyl fatty acids, ω-2 hydroxyl fatty acid, ω-3 hydroxyl fatty acid, and combinations thereof;
(iii) preparing a second reaction mixture comprising the hydroxyl fatty acids produced in step (ii) and a lipase; and
(iv) incubating the second reaction mixture for a sufficient time to produce the lactone.

In some embodiments, wherein step (ii) further comprises isolating the hydroxyl fatty acids from the first reaction mixture.

In some embodiments, the cytochrome P450 hydroxylase comprises an amino acid sequence that is at least 80% identical to the amino acid sequence of SEQ ID NO: 1 or SEQ ID NO: 3. In some embodiments, the cytochrome P450 hydroxylase comprises the amino acid sequence of SEQ ID NO: 1 or SEQ ID NO: 3.

In some embodiments, the lipase is lipase B from Candida antarctica. In some embodiments, the lipase comprises an amino acid sequence that is at least 80% identical to the amino acid sequence of SEQ ID NO: 5. In some embodiments, the lipase comprises the amino acid sequence of SEQ ID NO: 5. In some embodiments, the lipase is immobilized on a solid support. In some embodiments, the second reaction mixture further comprises a solvent, optionally wherein the solvent is toluene or dichloroethane. In some embodiments, the hydroxyl fatty acids in the second reaction mixture are at a total concentration of 0.02-0.1 M, optionally wherein the hydroxyl fatty acids are at a total concentration of 0.025-0.5 M. In some embodiments, the lipase in the second reaction mixture is at a concentration of 20-150 g/L, optionally wherein the lipase is at a concentration of 50-100 g/L. In some embodiments, step (iv) further comprises isolating the lactone.

In some embodiments, the one or more fatty acids of step (i) comprise a linear fatty acid comprising 12-28 carbon atoms, optionally wherein the one or more fatty acids of step (i) comprise a linear fatty acid comprising 15, 16, 17, 18, or 20 carbon atoms. In some embodiments, the one or more fatty acids of step (i) comprise a saturated fatty acid. In some embodiments, the one or more fatty acids of step (i) comprise an unsaturated fatty acid, optionally wherein the unsaturated fatty acid comprises at least one double bond, optionally wherein the unsaturated fatty acid comprises at least one Z double bond.

In some embodiments, the one or more fatty acids of step (i) are selected from the group consisting of:

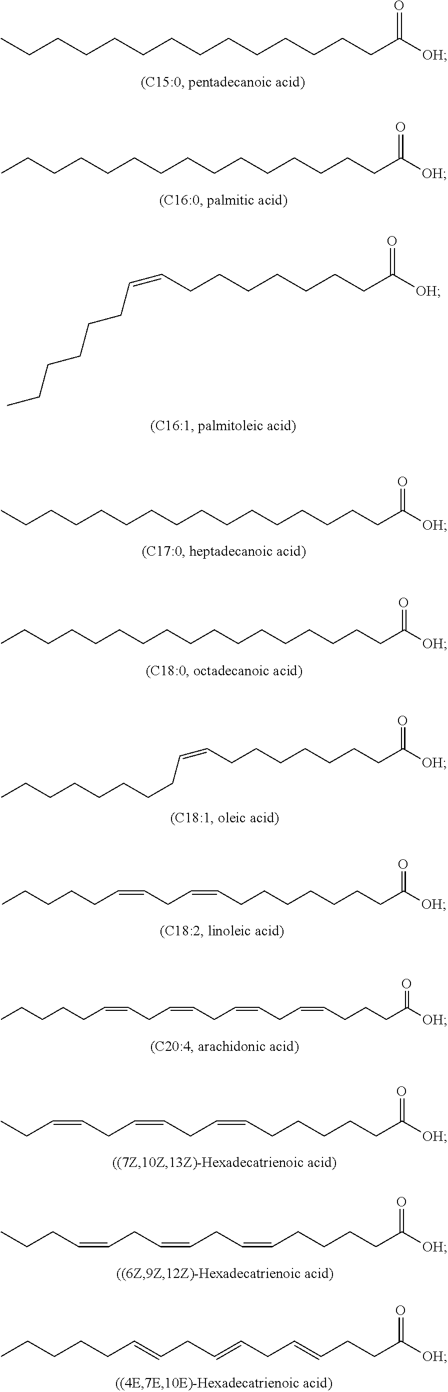

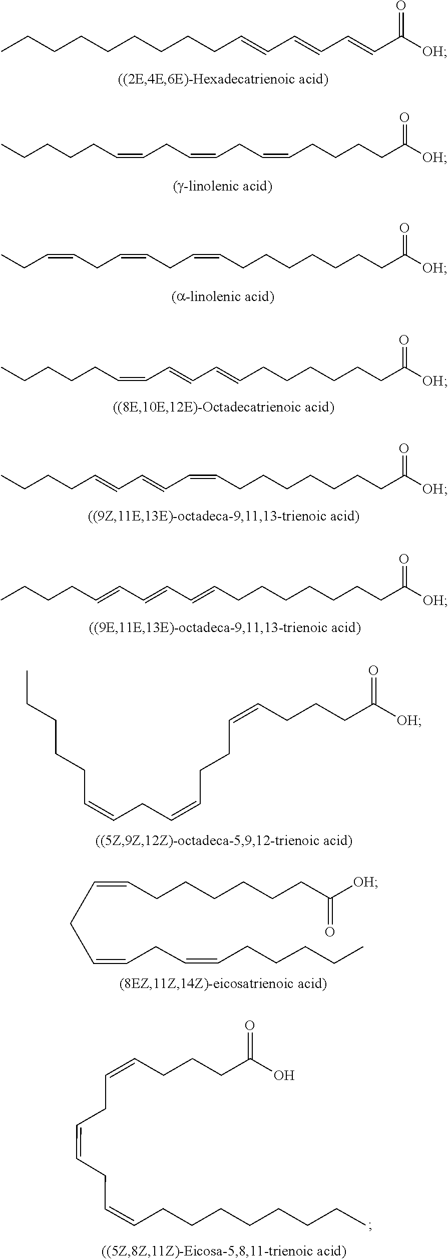

and combinations thereof.

In some embodiments, the one or more fatty acids of step (i) are selected from the group consisting of:

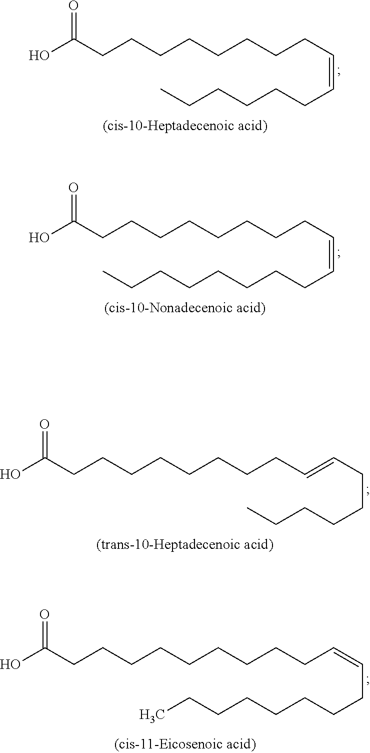 [SMILES: CCCCCC/C=C\CCCCCCCCC(=O)O ; CCCCCCCC/C=C\CCCCCCCCC(=O)O ; CCCCCC/C=C/CCCCCCCCC(=O)O ; CCCCCCCC/C=C\CCCCCCCCCC(=O)O]

and combinations thereof. In some embodiments, the lactone comprises one or more compounds of the formula:

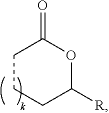

wherein:
   R is methyl, ethyl, or n-propyl;
   each [symbol] is independently a single bond, E double bond, Z double bond, or triple bond, as valency permits; and
   k is an integer between 6 and 30, inclusive.

In some embodiments, the lactone comprises one or more compounds of the formula:

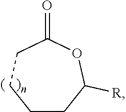

wherein:
   R is methyl, ethyl, or n-propyl;
   each [symbol] is independently a single bond, E double bond, Z double bond, or triple bond, as valency permits; and
   n is an integer between 6 and 20, inclusive.

In some embodiments, the one or more fatty acids of step (i) comprise

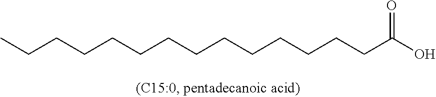 [SMILES: CCCCCCCCCCCCCCC(=O)O]

and the lactone produced in step (iv) comprises:

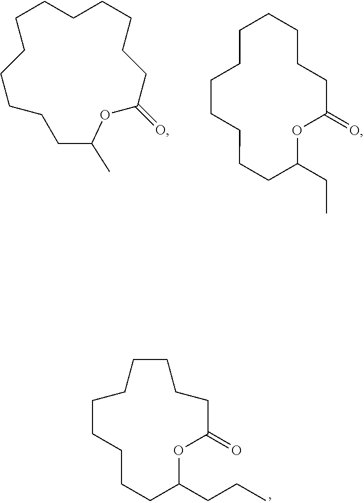 [SMILES: CC1CCCCCCCCCCCCC(=O)O1 ; CCC1CCCCCCCCCCCC(=O)O1 ; CCCC1CCCCCCCCCCC(=O)O1]

and combinations thereof.

In some embodiments, the one or more fatty acids of step (i) comprise

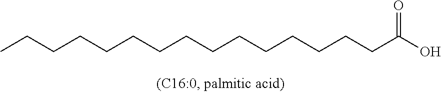 [SMILES: CCCCCCCCCCCCCCCC(=O)O]

and the lactone produced in step (iv) comprises:

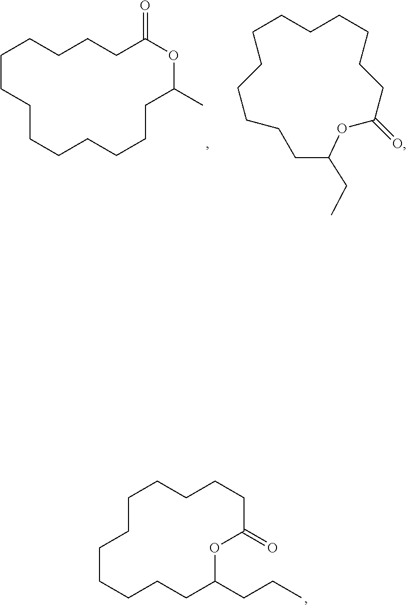

and combinations thereof.

In some embodiments, the one or more fatty acids of step (i) comprise

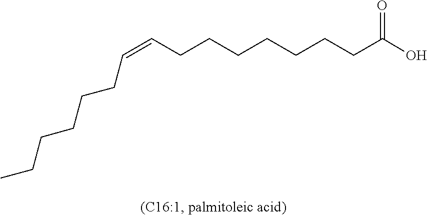 [SMILES: CCCCCC/C=C\CCCCCCCC(=O)O]

and the lactone produced in step (iv) comprises:

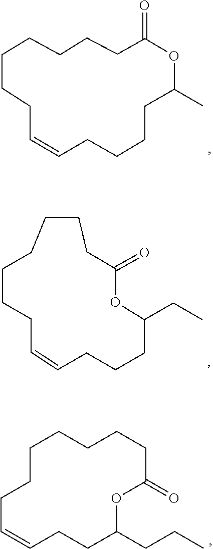 [SMILES: CC1CCCC/C=C\CCCCCCCC(=O)O1 ; CCC1CCC/C=C\CCCCCCCC(=O)O1 ; CCCC1CC/C=C\CCCCCCCC(=O)O1]

and combinations thereof.

In some embodiments, the one or more fatty acids of step (i) comprise

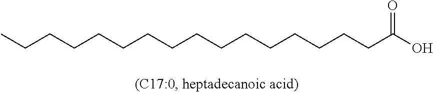 [SMILES: CCCCCCCCCCCCCCCCC(=O)O]

and the lactone produced in step (iv) comprises:

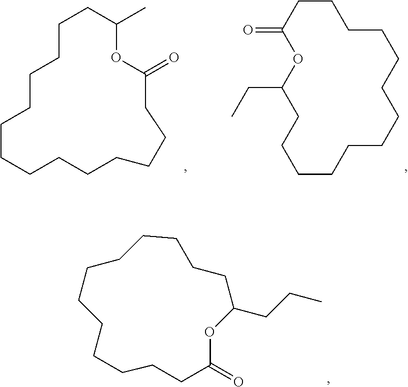 [SMILES: CC1CCCCCCCCCCCCCCC(=O)O1 ; CCC1CCCCCCCCCCCCCC(=O)O1 ; CCCC1CCCCCCCCCCCCC(=O)O1]

and combinations thereof.

In some embodiments, the one or more fatty acids of step (i) comprise

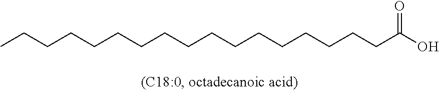 [SMILES: CCCCCCCCCCCCCCCCCC(=O)O]

and the lactone produced in step (iv) comprises:

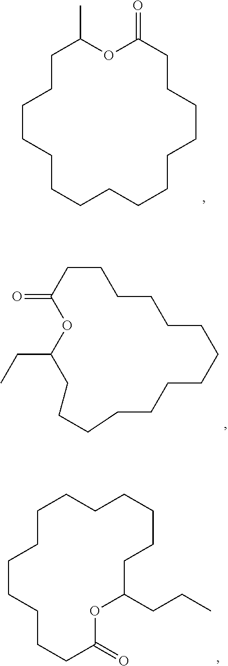 [SMILES: CC1CCCCCCCCCCCCCCCC(=O)O1 ; CCC1CCCCCCCCCCCCCCC(=O)O1 ; CCCC1CCCCCCCCCCCCCC(=O)O1]

and combinations thereof.

In some embodiments, the one or more fatty acids of step (i) comprise

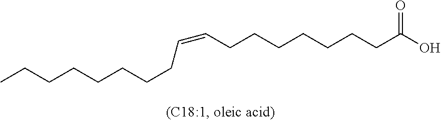 [SMILES: CCCCCCCC/C=C\CCCCCCCC(=O)O]

and the lactone produced in step (iv) comprises:

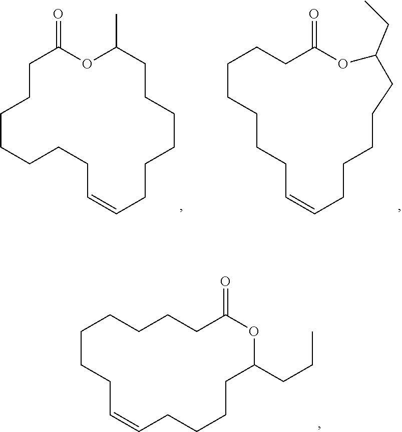 [SMILES: CC1CCCCCC/C=C\CCCCCCCC(=O)O1 ; CCC1CCCCC/C=C\CCCCCCCC(=O)O1 ; CCCC1CCCC/C=C\CCCCCCCC(=O)O1]

and combinations thereof.

In some embodiments, the one or more fatty acids of step (i) comprise

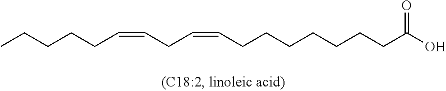 [SMILES: CCCCC/C=C\C/C=C\CCCCCCCC(=O)O]

and the lactone produced in step (iv) comprises:

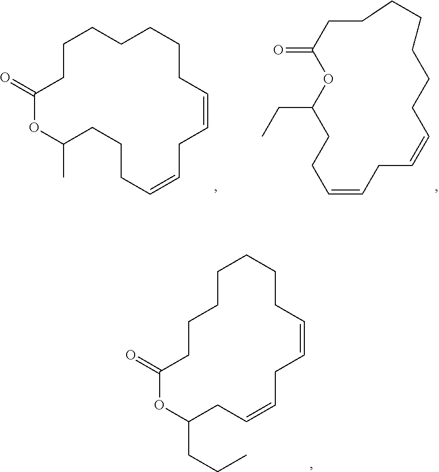 [SMILES: CC1CCC/C=C\C/C=C\CCCCCCCC(=O)O1 ; CCC1CC/C=C\C/C=C\CCCCCCCC(=O)O1 ; CCCC1C/C=C\C/C=C\CCCCCCCC(=O)O1]

and combinations thereof.

In some embodiments, the one or more fatty acids of step (i) comprise

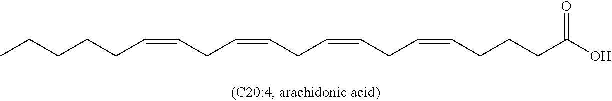 [SMILES: CCCCC/C=C\C/C=C\C/C=C\C/C=C\CCCC(=O)O]

and the lactone produced in step (iv) comprises:

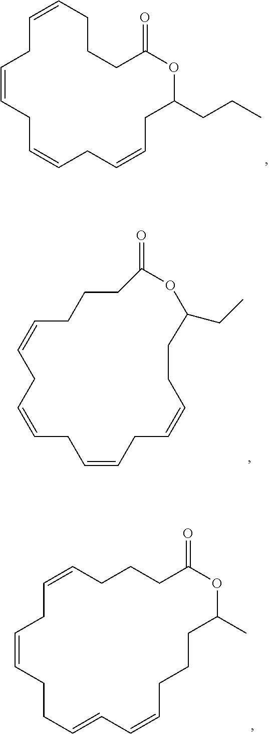 [SMILES: CCCC1C/C=C\C/C=C\C/C=C\C/C=C\CCCC(=O)O1 ; CCC1CC/C=C\C/C=C\C/C=C\C/C=C\CCCC(=O)O1 ; CC1CCC/C=C\C=C\CC/C=C\C/C=C\CCCC(=O)O1]

and combinations thereof.

In some embodiments, the one or more fatty acids used as substrates in the first reaction mixture in step (i) comprise

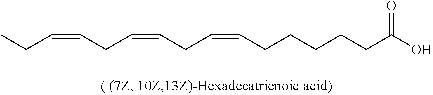 [SMILES: CC/C=C\C/C=C\C/C=C\CCCCCC(=O)O]

and the lactone produced in step (iv) comprises:

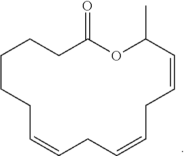 [SMILES: CC1/C=C\C/C=C\C/C=C\CCCCCC(=O)O1]

In some embodiments, the one or more fatty acids used as substrates in the first reaction mixture in step (i) comprise

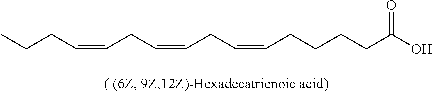 [SMILES: CCC/C=C\C/C=C\C/C=C\CCCCC(=O)O]

and the lactone produced in step (iv) comprises:

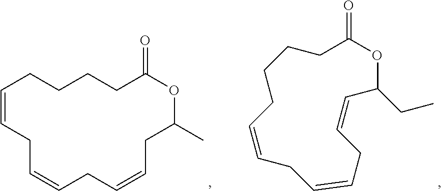 [SMILES: CC1C/C=C\C/C=C\C/C=C\CCCCC(=O)O1 ; CCC1/C=C\C/C=C\C/C=C\CCCCC(=O)O1]

and combinations thereof.

In some embodiments, the one or more fatty acids used as substrates in the first reaction mixture in step (i) comprise

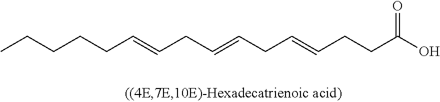 [SMILES: CCCCC/C=C/C/C=C/C/C=C/CCC(=O)O]

and the lactone produced in step (iv) comprises:

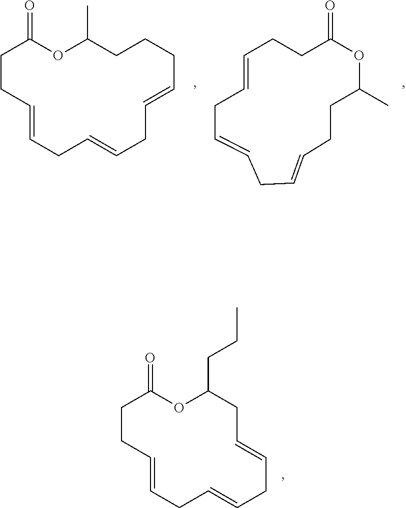

and combinations thereof.

In some embodiments, the one or more fatty acids used as substrates in the first reaction mixture in step (i) comprise

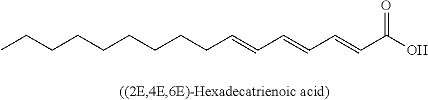 [SMILES: CCCCCCCCC/C=C/C=C/C=C/C(=O)O]

and the lactone produced in step (iv) comprises:

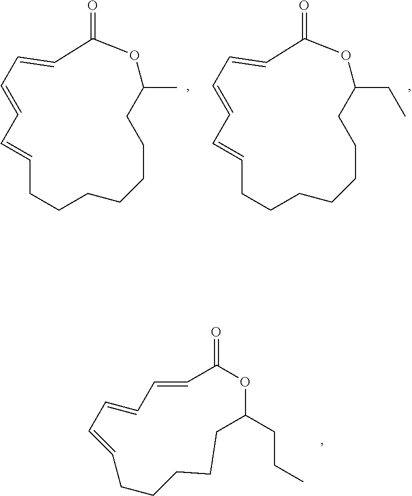

and combinations thereof.

In some embodiments, the one or more fatty acids used as substrates in the first reaction mixture in step (i) comprise

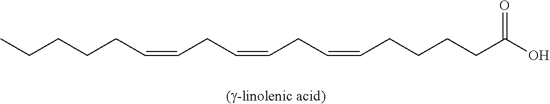 [SMILES: CCCCC/C=C\C/C=C\C/C=C\CCCCC(=O)O]

and the lactone produced in step (iv) comprises:

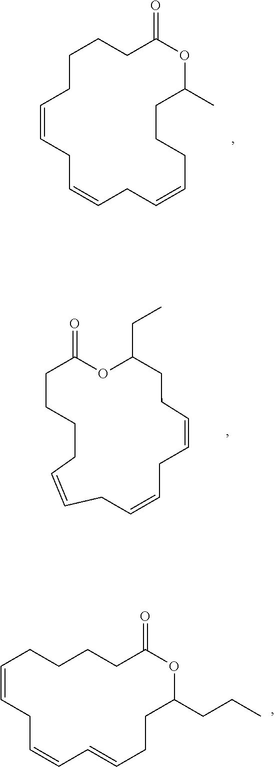 [SMILES: CC1CCC/C=C\C/C=C\C/C=C\CCCCC(=O)O1 ; CCC1CC/C=C\C/C=C\C/C=C\CCCCC(=O)O1 ; CCCC1CC/C=C/C=C\C/C=C\CCCCC(=O)O1]

and combinations thereof.

In some embodiments, the one or more fatty acids used as substrates in the first reaction mixture in step (i) comprise

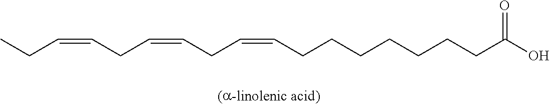 [SMILES: CC/C=C\C/C=C\C/C=C\CCCCCCCC(=O)O]

(α-linolenic acid) and the lactone produced in step (iv) comprises:

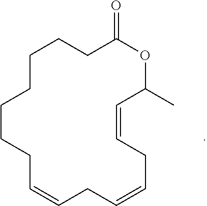 [SMILES: CC1/C=C\C/C=C\C/C=C\CCCCCCCC(=O)O1]

In some embodiments, the one or more fatty acids used as substrates in the first reaction mixture in step (i) comprise

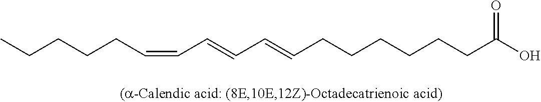 [SMILES: CCCCC\C=C/C=C/C=C/CCCCCCC(=O)O]

and the lactone produced in step (iv) comprises:

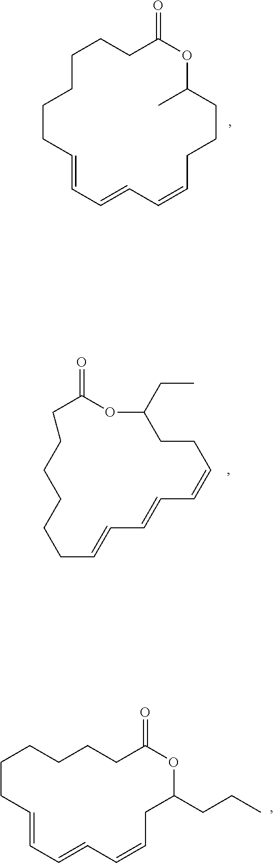 [SMILES: CC1CCC\C=C/C=C/C=C/CCCCCCC(=O)O1 ; CCC1CC\C=C/C=C/C=C/CCCCCCC(=O)O1 ; CCCC1C\C=C/C=C/C=C/CCCCCCC(=O)O1]

and combinations thereof.

In some embodiments, the one or more fatty acids used as substrates in the first reaction mixture in step (i) comprise

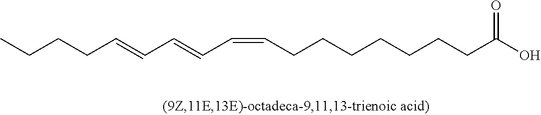 [SMILES: CCCC/C=C/C=C/C=C\CCCCCCCC(=O)O]

and the lactone produced in step (iv) comprises:

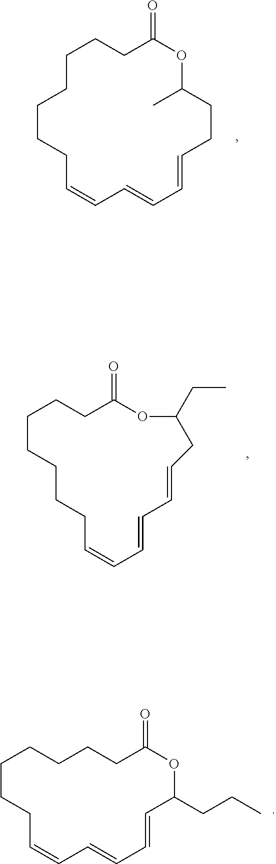 [SMILES: CC1CC/C=C/C=C/C=C\CCCCCCCC(=O)O1 ; CCC1C/C=C/C=C/C=C\CCCCCCCC(=O)O1 ; CCCC1/C=C/C=C/C=C\CCCCCCCC(=O)O1]

and combinations thereof.

In some embodiments, the one or more fatty acids used as substrates in the first reaction mixture in step (i) comprise

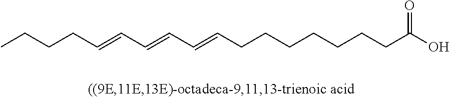 [SMILES: CCCC/C=C/C=C/C=C/CCCCCCCC(=O)O]

and the lactone produced in step (iv) comprises:

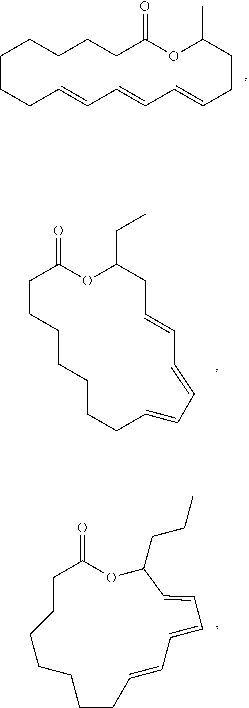 [SMILES: CC1CC/C=C/C=C/C=C/CCCCCCCC(=O)O1 ; CCC1C/C=C/C=C/C=C/CCCCCCCC(=O)O1 ; CCCC1/C=C/C=C/C=C/CCCCCCCC(=O)O1]

and combinations thereof.

In some embodiments, the one or more fatty acids used as substrates in the first reaction mixture in step (i) comprise

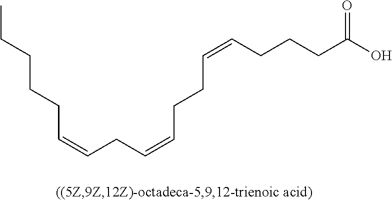 [SMILES: CCCCC/C=C\C/C=C\CC/C=C\CCCC(=O)O]

((5Z,9Z,12Z)-octadeca-5,9,12-trienoic acid) and the lactone produced in step (iv) comprises:

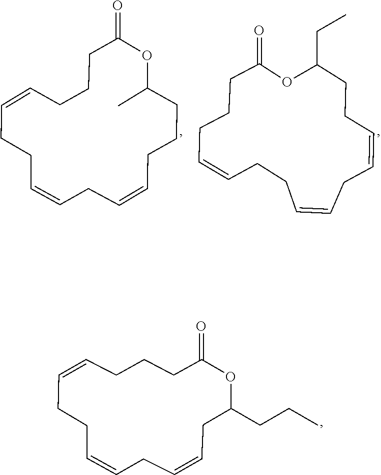

and combinations thereof.

In some embodiments, the one or more fatty acids used as substrates in the first reaction mixture in step (i) comprise

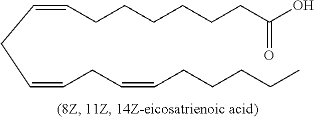 [SMILES: CCCCC/C=C\C/C=C\C/C=C\CCCCCCC(=O)O]

and the lactone produced in step (iv) comprises:

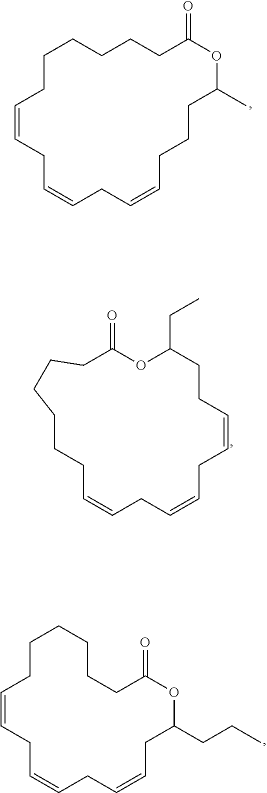 [SMILES: CC1CCC/C=C\C/C=C\C/C=C\CCCCCCC(=O)O1 ; CCC1CC/C=C\C/C=C\C/C=C\CCCCCCC(=O)O1 ; CCCC1C/C=C\C/C=C\C/C=C\CCCCCCC(=O)O1]

and combinations thereof.

In some embodiments, the one or more fatty acids used as substrates in the first reaction mixture in step (i) comprise

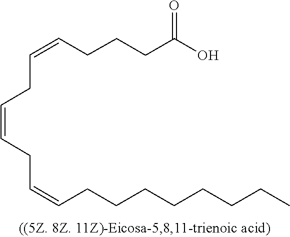 [SMILES: CCCCCCCC/C=C\C/C=C\C/C=C\CCCC(=O)O]

and the lactone produced in step (iv) comprises:

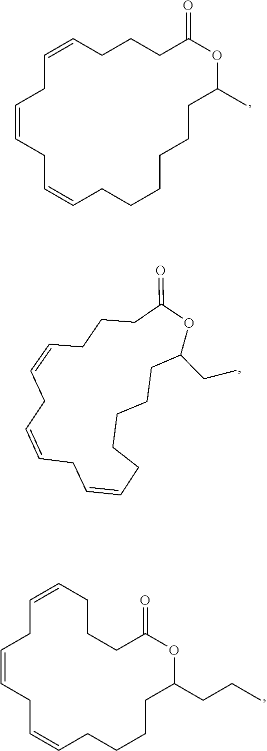 [SMILES: CC1CCCCCC/C=C\C/C=C\C/C=C\CCCC(=O)O1 ; CCC1CCCCC/C=C\C/C=C\C/C=C\CCCC(=O)O1 ; CCCC1CCCC/C=C\C/C=C\C/C=C\CCCC(=O)O1]

and combinations thereof.

In some embodiments, the one or more fatty acids used as substrates in the first reaction mixture in step (i) comprise

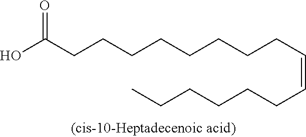 [SMILES: CCCCCC/C=C\CCCCCCCCC(=O)O]

and the lactone produced in step (iv) comprises:

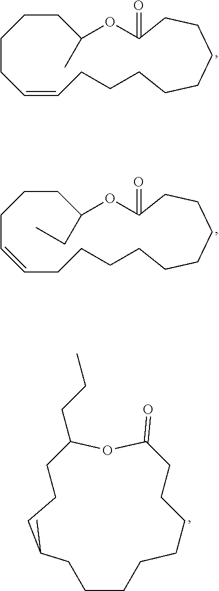 [SMILES: CC1CCCC/C=C\CCCCCCCCC(=O)O1 ; CCC1CCC/C=C\CCCCCCCCCC(=O)O1 ; CCCC1CCCC(C)CCCCCCCCC(=O)O1]

and combinations thereof.

In some embodiments, the one or more fatty acids used as substrates in the first reaction mixture in step (i) comprise

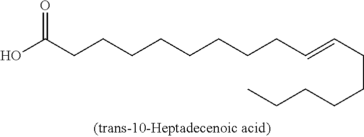 [SMILES: CCCCCC/C=C/CCCCCCCCC(=O)O]

and the lactone produced in step (iv) comprises:

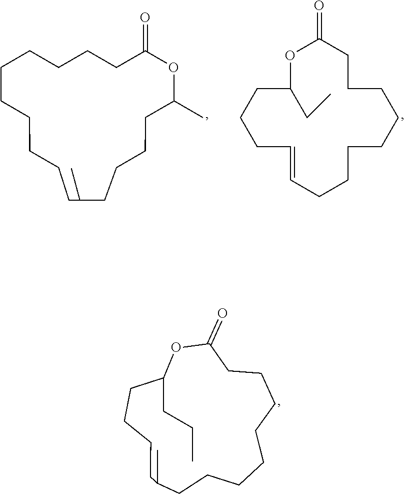

and combinations thereof.

In some embodiments, the one or more fatty acids used as substrates in the first reaction mixture in step (i) comprise

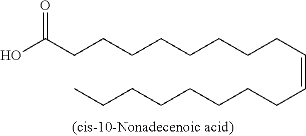 [SMILES: CCCCCCCC/C=C\CCCCCCCCC(=O)O]

and the lactone produced in step (iv) comprises:

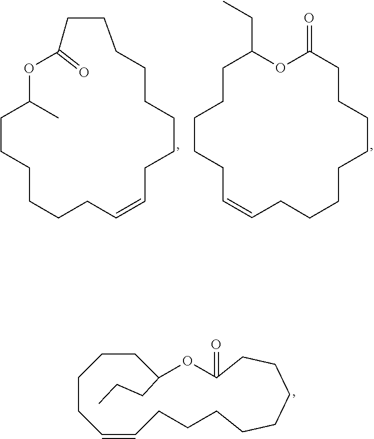

and combinations thereof.

In some embodiments, the one or more fatty acids used as substrates in the first reaction mixture in step (i) comprise

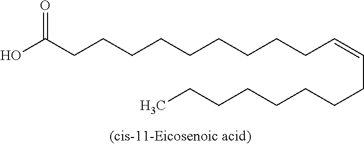 [SMILES: CCCCCCCC/C=C\CCCCCCCCCC(=O)O]

and the lactone produced in step (iv) comprises:

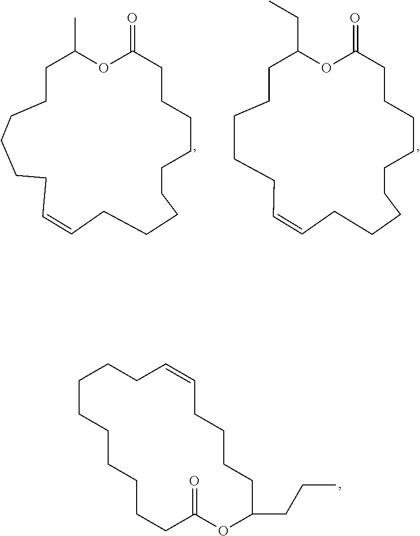

and combinations thereof.

In some embodiments, the first reaction mixture is in vitro. In some embodiments, the first reaction is a cell-based reaction mixture. In some embodiments, the cell-based reaction mixture comprises a cell selected from the group consisting of a yeast, a plant, an alga, a fungus, and a bacterium. In some embodiments, the cell-based reaction mixture comprises a bacterial cell of a genus selected from the group consisting of Escherichia; Salmonella; Bacillus; Acinetobacter; Corynebacterium; Methylosinus; Methylomonas; Rhodococcus; Pseudomonas; Rhodobacter; Synechocystis; Brevibacteria; Microbacterium; Arthrobacter; Citrobacter; Escherichia; Klebsiella; Pantoea; Salmonella; Corynebacterium; and Clostridium, optionally wherein the cell-based reaction mixture comprises an E. coli cell. In some embodiments, the cell-based reaction mixture comprises a fungus of a genus selected from the group consisting of Saccharomyces; Zygosaccharomyces; Kluyveromyces; Candida; Streptomyces; Hansenula; Debaryomyces; Mucor; Pichia; Torulopsis; Aspergillus; and Arthrobotlys.

In some embodiments, the lactone produced in step (iv) has a purity of at least 70%. In some embodiments, rein the lactone produced in step (iv) has musk notes.

Other aspects of the present disclosure provide lactones of the formula:

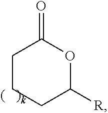

wherein:
   R is methyl, ethyl, or n-propyl;
   each [symbol] is independently a single bond, E double bond, Z double bond, or triple bond, as valency permits; and
   k is an integer between 6 and 30, inclusive;
   provided that the lactone is not of the formula:

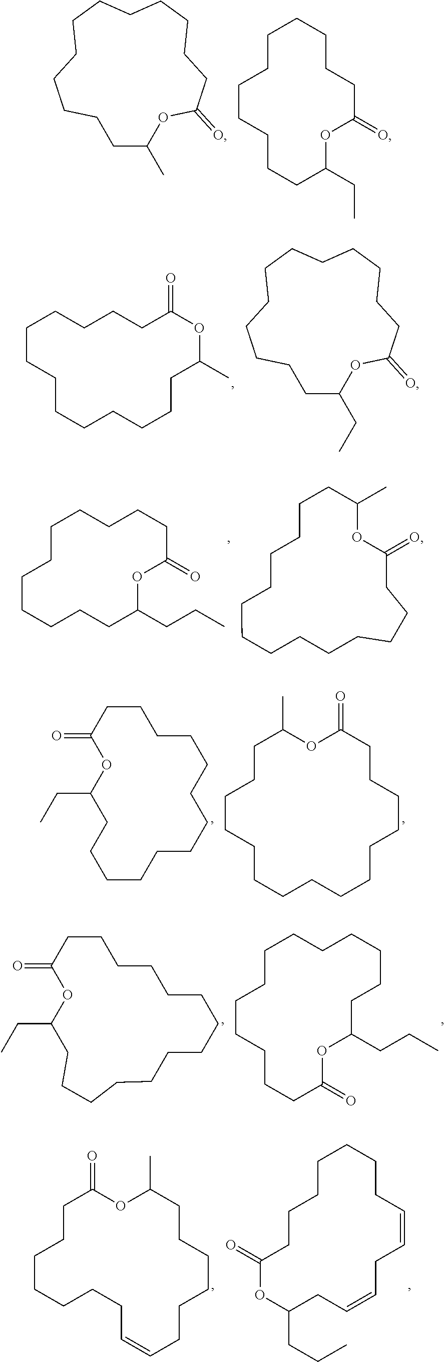

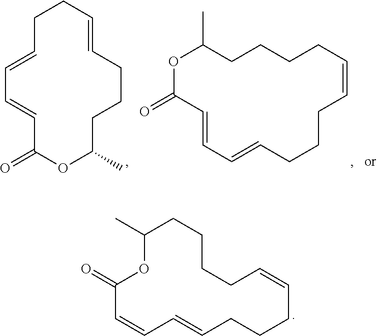

In some embodiments, the lactone is of the formula:

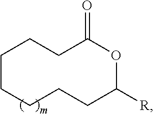

wherein:
   R is methyl, ethyl, or n-propyl;
   each [symbol] is independently a single bond or Z double bond, as valency permits, wherein 0, 1, 2, or 4 [symbol] are Z double bonds; and
   m is an integer between 4 and 11, inclusive;
   provided that the lactone is not of the formula:

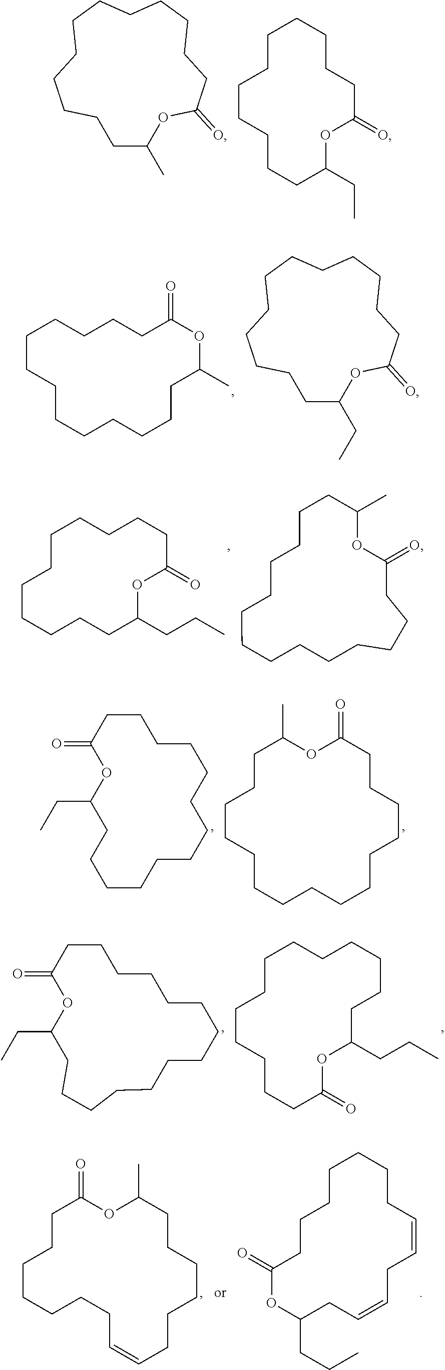

In some embodiments, each [symbol] is a single bond. In some embodiments, one [symbol] is an E or Z double bond, and the remining [symbol] are single bonds. In some embodiments, two [symbol] are independently E or Z double bonds, and the remining [symbol] are single bonds. In some embodiments, three [symbol] are independently E or Z double bonds, and the remining [symbol] are single bonds. In some embodiments, four [symbol] are independently E or Z double bonds, and the remining [symbol] are single bonds. In some embodiments, each double bond if present is a Z double bond. In some embodiments, each double bond if present is an E double bond. In some embodiments, the lactone does not comprise any one of C═C═C, C═C≡C, and C≡C═C. In some embodiments, k is 8, 9, 10, 11, 12, 13, or 15.

In some embodiments, the lactone is of the formula:

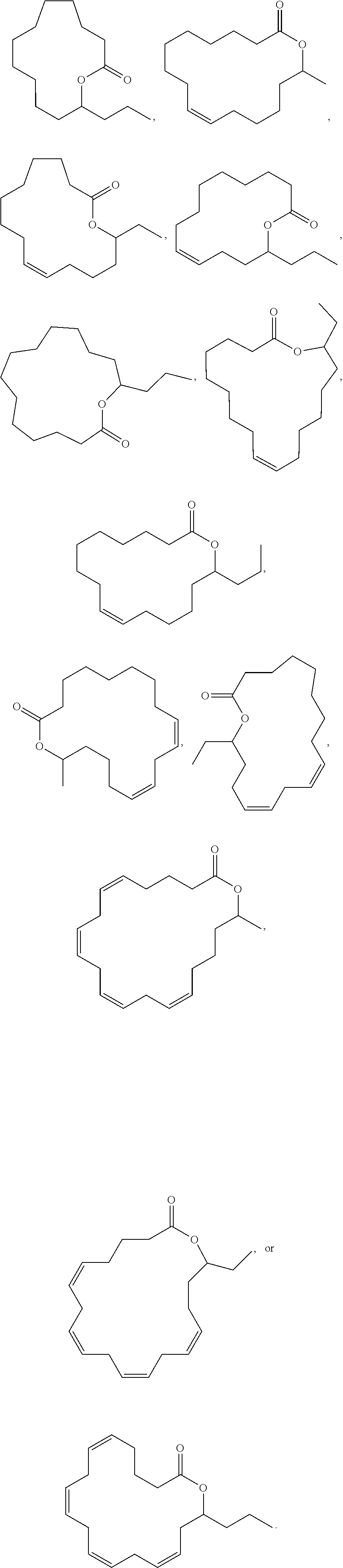

In some embodiments, the lactone is of the formula:

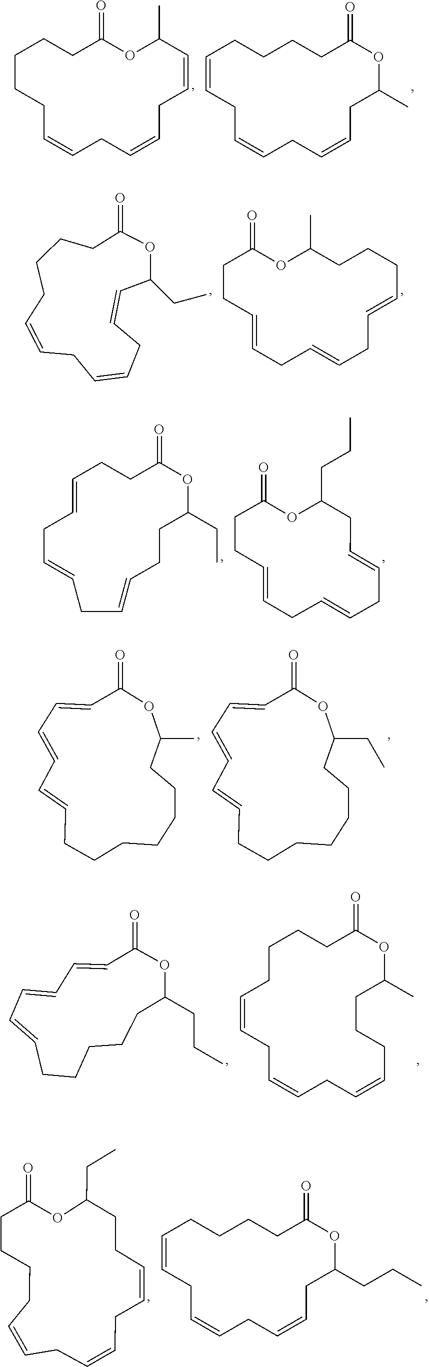

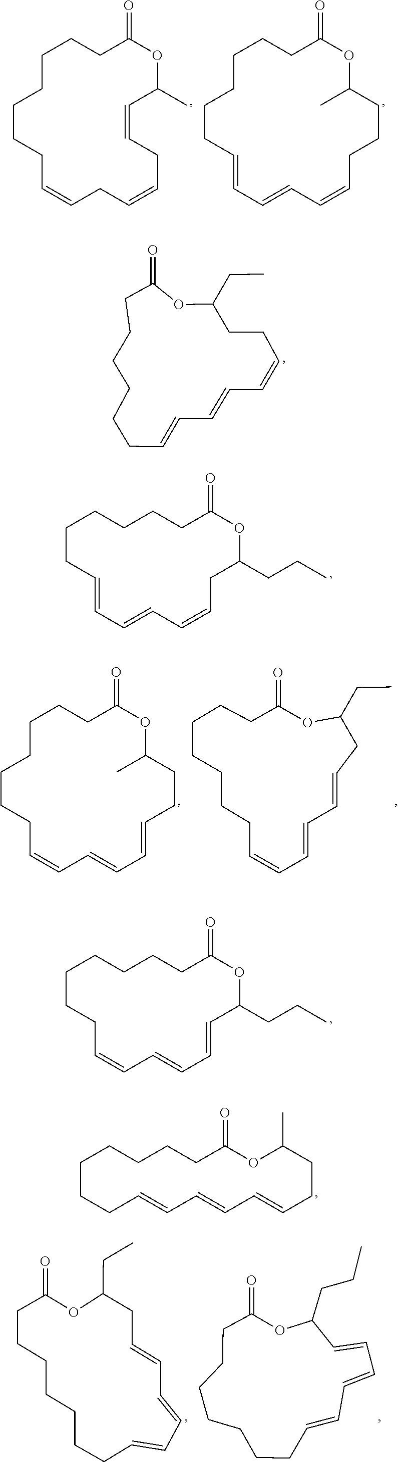

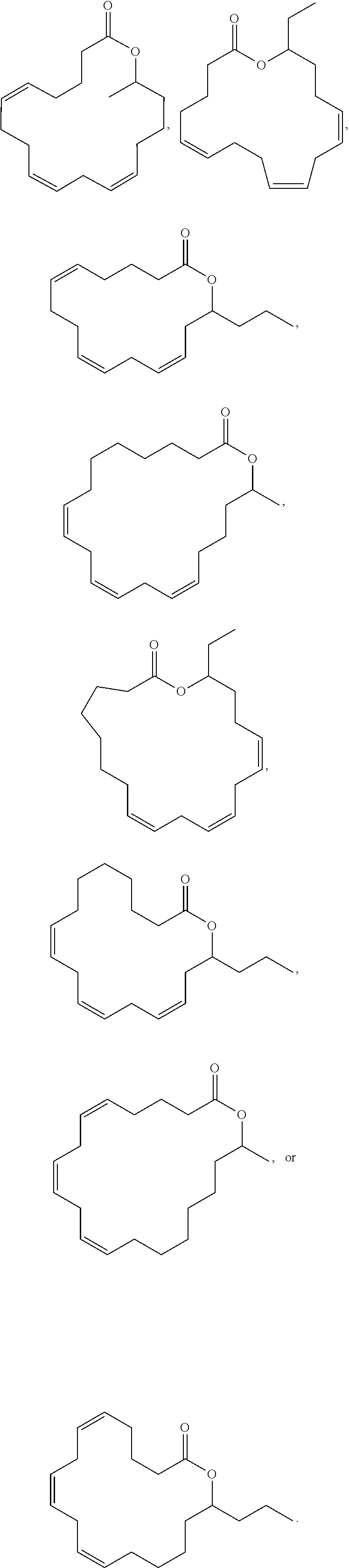

In some embodiments, the lactone is of the formula:

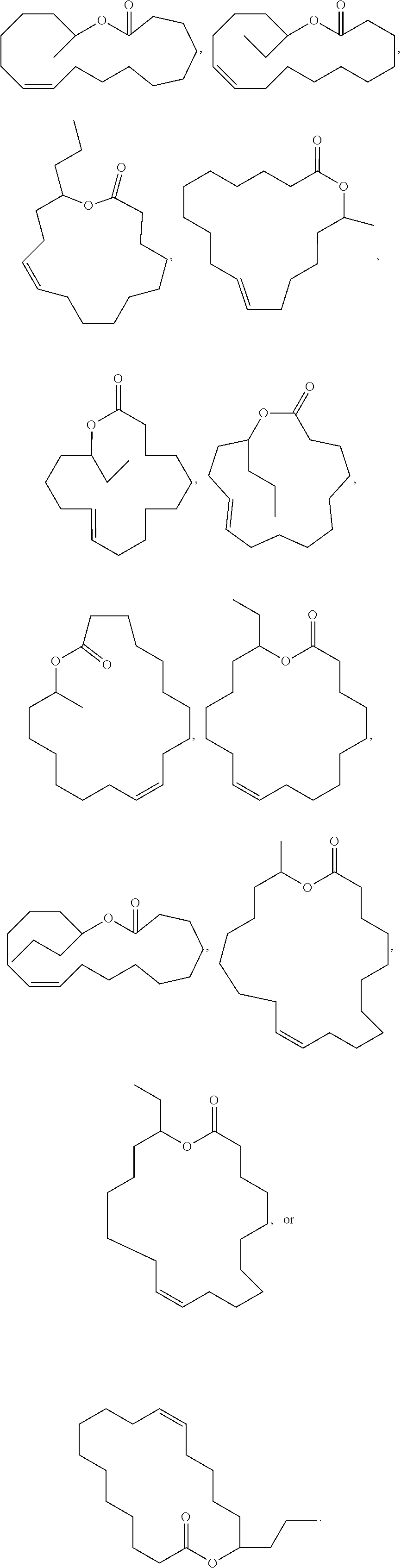

In some embodiments, the chiral carbon atom is of the S configuration. In some embodiments, the chiral carbon atom is of the R configuration.

Mixture of two or more lactones described herein and compositions comprising the lactones or mixtures of two or more lactones described herein are also provided. In some embodiments, the composition further comprising a cosmetically acceptable excipient.

Also provided herein are lactones produced by any one of the methods described herein.

While the disclosure is susceptible to various modifications and alternative forms, specific embodiments thereof are shown by way of example in the drawing and will herein be described in detail. It should be understood, however, that the drawings and detailed description presented herein are not intended to limit the disclosure to the particular embodiments disclosed, but on the contrary, the intention is to cover all modifications, equivalents, and alternatives falling within the spirit and scope of the present disclosure as defined by the appended claims.

Other features and advantages of this invention will become apparent in the following detailed description of preferred embodiments of this invention, taken with reference to the accompanying drawings.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1. Overall synthetic scheme showing fatty acid hydroxylation (Process 1) and a reaction of obtaining macrolactones (Process 2).

FIG. 2. GC/MS analysis of reaction products catalyzed by lipase. The numbers indicate the musk lactone peaks from different fatty acids. The molecular weights of these musky lactones match their calculated molecular weights.

FIGS. 3A-3F. Reaction schemes showing fatty acid hydroxylation and ring closure for oleic acid (FIG. 3A), linoleic acid (FIG. 3B), arachidonic acid (FIG. 3C), palmitic acid (FIG. 3D), heptadecanoic acid (FIG. 3E), and octadecanoic acid (FIG. 3F).

FIG. 4. Overall synthetic scheme showing palmitic acid hydroxylation (Process 1) and a reaction of obtaining corresponding musk lactone products (Process 2).

FIG. 5. Reaction showing hydroxyl oleic acid cyclized to obtain corresponding musk lactone products (Process 2).

FIG. 6. Chromatogram and spectrum showing analysis of the oleic acid macrolactonization products.

FIG. 7. Overall synthetic scheme showing linoleic acid hydroxylation (Process 1) and a reaction of obtaining corresponding musk lactone products (Process 2) is illustrated in FIG. 6.

FIG. 8. GC/MS showing analysis of the linoleic acid macrolactonization products.

FIG. 9. Production of musk lactones from hydroxy γ-linolenic acid (GLA, C18:3).

FIG. 10. GC/MS analysis of musk lactones derived from hydroxy GLA (molecular weight: 276).

FIG. 11. Production of musky lactone from α-linolenic acid (ALA, C18:3).

FIG. 12. GC/MS analysis of musk lactone from hydroxy ALA (molecular weight: 276).

FIG. 13. Production of musk lactone from 8Z,11Z,14Z-eicosatrienoic acid, dihomo-γ-linolenic acid (DGLA, C20:3).

FIG. 14. GC/MS analysis of musk lactone from hydroxy DGLA (molecular weight: 304).

FIG. 15. Reaction showing cis-10-Heptadecenoic acid (C17:1 cis-10) hydroxylation and cyclization to obtain corresponding musk lactone products.

FIG. 16. Reaction showing trans-10-Heptadecenoic acid (C17:1 trans-10) hydroxylation and cyclization to obtain corresponding musk lactone products.

FIG. 17. Reaction showing cis-10-Nonadecenoic acid (C19:1 cis-10) hydroxylation and cyclization to obtain corresponding musk lactone products

FIG. 18. Reaction showing cis-11-Eicosenoic acid (C20:1 cis-11) hydroxylation and cyclization to obtain corresponding musk lactone products

FIG. 19 GC/MS analysis of musk lactone from cis-10-Heptadecenoic acid (C17:1 cis-10).

FIG. 20 GC/MS analysis of musk lactone from trans-10-Heptadecenoic acid (C17:1 trans-10).

FIG. 21 GC/MS analysis of musk lactone from cis-10-Nonadecenoic acid (C19:1 cis-10).

FIG. 22 GM/MS analysis of musk lactone from cis-11-Eicosenoic acid (C20:1 cis-11).

### DEFINITIONS

The term “alkyl” refers to a radical of a branched or unbranched, saturated acyclic hydrocarbon group. In certain embodiments, alkyl is C₃₋₃₆ alkyl. In certain embodiments, alkyl is C₁₀₋₃₆ alkyl. In certain embodiments, alkyl is C₁₋₂₇ alkyl. Unless otherwise provided, alkyl is C₃₋₂₉ alkyl.

The term “alkenyl” refers to a radical of a branched or unbranched, acyclic hydrocarbon group having one or more carbon-carbon double bonds (C═C bonds; e.g., 1, 2, 3, 4, 5, or 6 C═C bonds), as valency permits. In alkenyl groups,

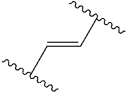

is an E double bond,

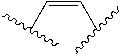 [SMILES: */C=C\*]

is an Z double bond. Other situations involving an E or Z double bond are as known in the art. In an alkenyl group, a C═C bond for which the stereochemistry is not specified (e.g., —CH═CH— or

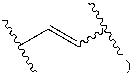

may be a E or Z double bond. In certain embodiments, alkenyl is C₃₋₃₆ alkenyl. In certain embodiments, alkenyl is C₁₀₋₃₆ alkenyl. In certain embodiments, alkenyl is C₁₁₋₂₇ alkenyl. Unless otherwise provided, alkenyl is C₃₋₂₉ alkenyl.

The term “alkynyl” refers to a radical of a branched or unbranched, acyclic hydrocarbon group having one or more carbon-carbon triple bonds (C≡C bonds; e.g., 1, 2, 3, or 4 triple bonds), as valency permits. In certain embodiments, alkynyl is C₃₋₃₆ alkynyl. In certain embodiments, alkynyl is C₁₀₋₃₆ alkynyl. In certain embodiments, alkynyl is C₁₁₋₂₇ alkynyl. Unless otherwise provided, alkynyl is C₃₋₂₉ alkynyl.

Affixing the suffix “ene” to a group indicates the group is a divalent moiety, e.g., alkylene is a divalent moiety of alkyl (e.g., C₃₋₃₆ alkyl, C₁₁₋₃₆ alkyl, C₁₋₂₇ alkyl, or C₃₋₂₉ alkyl), alkenylene is a divalent moiety of alkenyl (e.g., C₃₋₃₆ alkenyl, C₁₀₋₃₆ alkenyl, C₁₁₋₂₇ alkenyl, or C₃₋₂₉ alkenyl), and alkynylene is a divalent moiety of alkynyl (e.g., C₃₋₃₆ alkynyl, C₁₀₋₃₆ alkynyl, C₁₋₂₇ alkynyl, or C₃₋₂₉ alkynyl).

A “fatty acid” is a carboxylic acid of the formula: R^{A}—C(═O)OH, wherein R^{A} is C₃₋₃₆ alkyl, C₃₋₃₆ alkenyl, or C₃₋₃₆ alkynyl (e.g., C₃₋₂₉ alkyl, C₃₋₂₉ alkenyl, or C₃₋₂₉ alkynyl). The carbon atom (e.g., C1) in R^{A} that is farthest to the carboxyl moiety is labelled as ω (omega). The carbon atom (e.g., C2) next to C1 is labelled as ω-1. The carbon atom (e.g., C3) that is next to C2 and is not C1 is labelled as ω-2. The carbon atom (e.g., C4) that is next to C3 and is not C2 is labelled as ω-3. A 15:0 fatty acid is a fatty acid where the number of carbon atoms is 15, and the number of C═C and C≡C bonds is 0. A 16:0 fatty acid is a fatty acid where the number of carbon atoms is 16, and the number of C═C and C≡C bonds is 0. A 16:1 fatty acid is a fatty acid where the number of carbon atoms is 16, the number of C═C bonds is 1, and the number of C≡C bonds is 0. A 16:3 fatty acid is a fatty acid where the number of carbon atoms is 16, the number of C═C bonds is 3, and the number of C≡C bonds is 0. A 17:0 fatty acid is a fatty acid where the number of carbon atoms is 17, and the number of C═C and C≡C bonds is 0. An 18:0 fatty acid is a fatty acid where the number of carbon atoms is 18, and the number of C═C and C≡C bonds is 0. An 18:1 fatty acid is a fatty acid where the number of carbon atoms is 18, the number of C═C bonds is 1, and the number of C≡C bonds is 0. An 18:2 fatty acid is a fatty acid where the number of carbon atoms is 18, the number of C≡C bonds is 2, and the number of C≡C bonds is 0. An 18:3 fatty acid is a fatty acid where the number of carbon atoms is 18, the number of C═C bonds is 3, and the number of C≡C bonds is 0. A 20:3 fatty acid is a fatty acid where the number of carbon atoms is 20, the number of C═C bonds is 3, and the number of C≡C bonds is 0. A 20:4 fatty acid is a fatty acid where the number of carbon atoms is 20, the number of C═C bonds is 4, and the number of C≡C bonds is 0. Other fatty acids can be named in this manner.

A “saturated fatty acid” is a fatty acid where R^{A} is C₃₋₃₆ alkyl (e.g., C₃₋₂₉ alkyl).

An “unsaturated fatty acid” is a fatty acid where R^{A} is C₃₋₃₆ alkenyl or C₃₋₃₆ alkynyl (e.g., C₃₋₂₉ alkenyl or C₃₋₂₉ alkynyl).

The term “hydroxyl” or “hydroxy” refers to the group —OH.

A “hydroxyl fatty acid,” “hydroxy fatty acid,” or “hydroxylated fatty acid” is a fatty acid where one or more hydrogen atom is replaced with hydroxyl. In some embodiments, the hydroxyl fatty acid is a fatty acid where one hydrogen atom is replaced with hydroxyl (mono-hydroxyl fatty acid). In some embodiments, the hydroxyl fatty acid is a fatty acid where more than one (e.g., 2, 3, or more) hydrogen atom is replaced with hydroxyl (poly-hydroxyl fatty acid).

A “lactone” is a monocyclic compound where the moiety —C(═O)O— is part of the monocyclic ring, and the remaining part of the monocyclic compound is alkylene, alkenylene, or alkynylene. When the alkylene, alkenylene, or alkynylene is branched, the lactone also includes the branch(es) of the alkylene, alkenylene, or alkynylene.

“Cellular system” is any cells that provide for the expression of ectopic proteins. It includes bacteria, yeast, plant cells and animal cells. It may include prokaryotic or eukaryotic host cells which are modified to express a recombinant protein and cultivated in an appropriate culture medium. It also includes the in vitro expression of proteins based on cellular components, such as ribosomes.

“Coding sequence” is to be given its ordinary and customary meaning to a person of ordinary skill in the art, and is used without limitation to refer to a DNA sequence that encodes for a specific amino acid sequence.

“Growing the Cellular System”. Growing includes providing an appropriate medium that would allow cells to multiply and divide, to form a cell culture. It also includes providing resources so that cells or cellular components can translate and make recombinant proteins.

“Protein Expression”. Protein production can occur after gene expression. It consists of the stages after DNA has been transcribed to messenger RNA (mRNA). The mRNA is then translated into polypeptide chains, which are ultimately folded into proteins. DNA or RNA may be present in the cells through transfection—a process of deliberately introducing nucleic acids into cells. The term is often used for non-viral methods in eukaryotic cells. It may also refer to other methods and cell types, although other terms are preferred: “transformation” is more often used to describe non-viral DNA transfer in bacteria, non-animal eukaryotic cells, including plant cells. In animal cells, transfection is the preferred term as transformation is also used to refer to progression to a cancerous state (carcinogenesis) in these cells. Transduction is often used to describe virus-mediated DNA transfer. Transformation, transduction, and viral infection are included under the definition of transfection for this application.

“Yeast”. According to the current disclosure a yeast are eukaryotic, single-celled microorganisms classified as members of the fungus kingdom. Yeasts are unicellular organisms which are believed to have evolved from multicellular ancestors.

As used herein, the singular forms “a, an” and “the” include plural references unless the content clearly dictates otherwise.

To the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments.

The term “complementary” is to be given its ordinary and customary meaning to a person of ordinary skill in the art and is used without limitation to describe the relationship between nucleotide bases that are capable to hybridizing to one another. For example, with respect to DNA, adenosine is complementary to thymine and cytosine is complementary to guanine. Accordingly, the subject technology also includes isolated nucleic acid fragments that are complementary to the complete sequences as reported in the accompanying Sequence Listing as well as those substantially similar nucleic acid sequences.

The terms “nucleic acid” and “nucleotide” are to be given their respective ordinary and customary meanings to a person of ordinary skill in the art and are used without limitation to refer to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally-occurring nucleotides. Unless otherwise indicated, a particular nucleic acid sequence also implicitly encompasses conservatively modified or degenerate variants thereof (e.g., degenerate codon substitutions) and complementary sequences, as well as the sequence explicitly indicated.

The term “isolated” is to be given its ordinary and customary meaning to a person of ordinary skill in the art, and when used in the context of an isolated nucleic acid or an isolated polypeptide, is used without limitation to refer to a nucleic acid or polypeptide that, by the hand of man, exists apart from its native environment and is therefore not a product of nature. An isolated nucleic acid or polypeptide can exist in a purified form or can exist in a non-native environment such as, for example, in a transgenic host cell.

The terms “incubating” and “incubation” as used herein means a process of mixing two or more chemical or biological entities (such as a chemical compound and an enzyme) and allowing them to interact under conditions favorable for producing a 6-lactone composition.

The term “degenerate variant” refers to a nucleic acid sequence having a residue sequence that differs from a reference nucleic acid sequence by one or more degenerate codon substitutions. Degenerate codon substitutions can be achieved by generating sequences in which the third position of one or more selected (or all) codons is substituted with mixed base and/or deoxyinosine residues. A nucleic acid sequence and all of its degenerate variants will express the same amino acid or polypeptide.

The terms “polypeptide,” “protein,” and “peptide” are to be given their respective ordinary and customary meanings to a person of ordinary skill in the art; the three terms are sometimes used interchangeably, and are used without limitation to refer to a polymer of amino acids, or amino acid analogs, regardless of its size or function. Although “protein” is often used in reference to relatively large polypeptides, and “peptide” is often used in reference to small polypeptides, usage of these terms in the art overlaps and varies. The term “polypeptide” as used herein refers to peptides, polypeptides, and proteins, unless otherwise noted. The terms “protein,” “polypeptide,” and “peptide” are used interchangeably herein when referring to a polyaminoacid product. Thus, exemplary polypeptides include polyaminoacid products, naturally occurring proteins, homologs, orthologs, paralogs, fragments and other equivalents, variants, and analogs of the foregoing.

The terms “polypeptide fragment” and “fragment,” when used in reference to a reference polypeptide, are to be given their ordinary and customary meanings to a person of ordinary skill in the art and are used without limitation to refer to a polypeptide in which amino acid residues are deleted as compared to the reference polypeptide itself, but where the remaining amino acid sequence is usually identical to the corresponding positions in the reference polypeptide. Such deletions can occur at the amino-terminus or carboxy-terminus of the reference polypeptide, or alternatively both.

The term “functional fragment” of a polypeptide or protein refers to a peptide fragment that is a portion of the full-length polypeptide or protein, and has substantially the same biological activity, or carries out substantially the same function as the full-length polypeptide or protein (e.g., carrying out the same enzymatic reaction).

The terms “variant polypeptide,” “modified amino acid sequence” or “modified polypeptide,” which are used interchangeably, refer to an amino acid sequence that is different from the reference polypeptide by one or more amino acids, e.g., by one or more amino acid substitutions, deletions, and/or additions. In an aspect, a variant is a “functional variant” which retains some or all of the ability of the reference polypeptide.

The term “functional variant” further includes conservatively substituted variants. The term “conservatively substituted variant” refers to a peptide having an amino acid sequence that differs from a reference peptide by one or more conservative amino acid substitutions and maintains some or all of the activity of the reference peptide. A “conservative amino acid substitution” is a substitution of an amino acid residue with a functionally similar residue. Examples of conservative substitutions include the substitution of one non-polar (hydrophobic) residue such as isoleucine, valine, leucine or methionine for another; the substitution of one charged or polar (hydrophilic) residue for another such as between arginine and lysine, between glutamine and asparagine, between threonine and serine; the substitution of one basic residue such as lysine or arginine for another; or the substitution of one acidic residue, such as aspartic acid or glutamic acid for another; or the substitution of one aromatic residue, such as phenylalanine, tyrosine, or tryptophan for another. Such substitutions are expected to have little or no effect on the apparent molecular weight or isoelectric point of the protein or polypeptide. The phrase “conservatively substituted variant” also includes peptides wherein a residue is replaced with a chemically-derivatized residue, provided that the resulting peptide maintains some or all of the activity of the reference peptide as described herein.

The term “variant,” in connection with the polypeptides of the subject technology, further includes a functionally active polypeptide having an amino acid sequence at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, and even 100% identical to the amino acid sequence of a reference polypeptide.

The term “homologous” in all its grammatical forms and spelling variations refers to the relationship between polynucleotides or polypeptides that possess a “common evolutionary origin,” including polynucleotides or polypeptides from super-families and homologous polynucleotides or proteins from different species (Reeck et al., CELL 50:667, 1987). Such polynucleotides or polypeptides have sequence homology, as reflected by their sequence similarity, whether in terms of percent identity or the presence of specific amino acids or motifs at conserved positions. For example, two homologous polypeptides can have amino acid sequences that are at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 900 at least 91%, at least 92%, at least 93%, at least 94%, at least 95%, at least 96%, at least 97%, at least 98%, at least 99%, and even 100% identical.

“Suitable regulatory sequences” is to be given its ordinary and customary meaning to a person of ordinary skill in the art, and is used without limitation to refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, and polyadenylation recognition sequences.

“Promoter” is to be given its ordinary and customary meaning to a person of ordinary skill in the art, and is used without limitation to refer to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. Promoters which cause a gene to be expressed in most cell types at most times, are commonly referred to as “constitutive promoters.” It is further recognized that since in most cases the exact boundaries of regulatory sequences have not been completely defined, DNA fragments of different lengths may have identical promoter activity.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “expression” as used herein, is to be given its ordinary and customary meaning to a person of ordinary skill in the art, and is used without limitation to refer to the transcription and stable accumulation of sense (mRNA) or antisense RNA derived from the nucleic acid fragment of the subject technology. “Over-expression” refers to the production of a gene product in transgenic or recombinant organisms that exceeds levels of production in normal or non-transformed organisms.

“Transformation” is to be given its ordinary and customary meaning to a person of reasonable skill in the field, and is used without limitation to refer to the transfer of a polynucleotide into a target cell for further expression by that cell. The transferred polynucleotide can be incorporated into the genome or chromosomal DNA of a target cell, resulting in genetically stable inheritance, or it can replicate independent of the host chromosomal DNA. Host organisms containing the transformed nucleic acid fragments are referred to as “transgenic” or “recombinant” or “transformed” organisms.

The terms “transformed,” “transgenic,” and “recombinant,” when used herein in connection with host cells, are to be given their respective ordinary and customary meanings to a person of ordinary skill in the art, and are used without limitation to refer to a cell of a host organism, such as a plant or microbial cell, into which a heterologous nucleic acid molecule has been introduced. The nucleic acid molecule can be stably integrated into the genome of the host cell, or the nucleic acid molecule can be present as an extrachromosomal molecule. Such an extrachromosomal molecule can be auto-replicating. Transformed cells, tissues, or subjects are understood to encompass not only the end product of a transformation process, but also transgenic progeny thereof.

The terms “recombinant,” “heterologous,” and “exogenous,” when used herein in connection with polynucleotides, are to be given their ordinary and customary meanings to a person of ordinary skill in the art, and are used without limitation to refer to a polynucleotide (e.g., a DNA sequence or a gene) that originates from a source foreign to the particular host cell or, if from the same source, is modified from its original form. Thus, a heterologous gene in a host cell includes a gene that is endogenous to the particular host cell but has been modified through, for example, the use of site-directed mutagenesis or other recombinant techniques. The terms also include non-naturally occurring multiple copies of a naturally occurring DNA sequence. Thus, the terms refer to a DNA segment that is foreign or heterologous to the cell, or homologous to the cell but in a position or form within the host cell in which the element is not ordinarily found.

Similarly, the terms “recombinant,” “heterologous,” and “exogenous,” when used herein in connection with a polypeptide or amino acid sequence, means a polypeptide or amino acid sequence that originates from a source foreign to the particular host cell or, if from the same source, is modified from its original form. Thus, recombinant DNA segments can be expressed in a host cell to produce a recombinant polypeptide.

The terms “plasmid,” “vector,” and “cassette” are to be given their respective ordinary and customary meanings to a person of ordinary skill in the art and are used without limitation to refer to an extra chromosomal element often carrying genes which are not part of the central metabolism of the cell, and usually in the form of circular double-stranded DNA molecules. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. “Transformation cassette” refers to a specific vector containing a foreign gene and having elements in addition to the foreign gene that facilitate transformation of a particular host cell. “Expression cassette” refers to a specific vector containing a foreign gene and having elements in addition to the foreign gene that allow for enhanced expression of that gene in a foreign host.

Unless defined otherwise, all technical and scientific terms used herein have the same meaning as commonly understood by one of ordinary skill in the art to which the disclosure belongs. Although any methods and materials similar to or equivalent to those described herein may be used in the practice or testing of the present disclosure, the preferred materials and methods are described below.

### DETAILED DESCRIPTION

Provided herein, in some aspects, are methods (e.g., biosynthetic methods) for the production of lactones, such as macrocyclic lactones, from fatty acids (e.g., linear fatty acids). In some embodiments, the lactones, such as macrocyclic lactones, produced using the methods described herein have differentiated musky notes (also referred to herein as “musk lactones” or “musky lactones”). In some embodiments, the macrocyclic lactones produced using the methods described herein comprise methyl, ethyl or propyl side chains. In some embodiments, the lactones, such as macrocyclic lactones, are produced using the methods described herein from saturated fatty acids (having no carbon-carbon double bonds) or unsaturated fatty acids (e.g., having one or more carbon-carbon double bonds). As described herein, Cytochrome P450 enzymes that have hydroxylase activity can carry out the first step of the biosynthetic method described herein, i.e., converting fatty acids to ω-1, ω-2, ω-3 hydroxyl fatty acids, or combinations thereof. The second step of the biosynthetic methods described herein, the macrolactonization of the ω-1, ω-2, ω-3 hydroxyl fatty acids, can be carried out by a lipase. Other aspects of the present disclosure provide lactones, such as macrocyclic lactones, with differentiated musky notes, which are produced using the methods described herein. In some embodiments, the lactones, such as macrocyclic lactones, produced herein comprise one or more compounds of the formula:

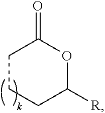

wherein:
   R is methyl, ethyl, or n-propyl;
   each [symbol] is independently a single bond, E double bond, Z double bond, or triple bond, as valency permits; and
   k is an integer between 6 and 30, inclusive.

In some embodiments, the lactones, such as macrocyclic lactones, produced herein comprise a structure of:

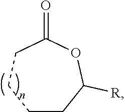

wherein:
   R is methyl, ethyl, or n-propyl;
   each [symbol] is independently a single bond, E double bond, Z double bond, or triple bond, as valency permits; and
   n is an integer between 6 and 20, inclusive.

### Biosynthetic Methods for Producing Lactones

The biosynthetic methods described herein utilize fatty acids (e.g., linear fatty acids) as substrates for enzymatic production of lactones, such as macrocyclic lactones (e.g., macrocyclic lactones having differentiated musky notes). The methods described herein comprise two enzymatic conversion steps. In the first step, the fatty acids are hydroxylated at ω-1, ω-2, or ω-3 positions by a cytochrome P450 enzyme having hydroxylase activity, or functional variants thereof to produce hydroxyl fatty acids (e.g., ω-1 hydroxyl fatty acids, ω-2 hydroxyl fatty acid, ω-3 hydroxyl fatty acid, and combinations thereof). In the second step, the hydroxyl fatty acids are subjected to macrocyclization by a lipase (e.g., Novozyme 435) to produce lactones, such as macrocyclic lactones, with methyl, ethyl, or propyl side chains. Without being bound to any particular theory, it is believed that either the macrocyclic rings, or the side chains, or both moieties impart the musky notes which are useful for industrial applications. The lactones, such as macrocyclic lactones, produced using the methods described herein that have musky notes are referred to as “musk lactones.”

In some embodiments, the biosynthetic methods of producing lactones (e.g., macrocyclic lactones) comprises: (i) preparing a first reaction mixture comprising one or more fatty acids, a cytochrome P450 hydroxylase, and NADPH; (ii) incubating the first reaction mixture of for a sufficient time to produce hydroxyl fatty acids selected from ω-1 hydroxyl fatty acids, ω-2 hydroxyl fatty acids, ω-3 hydroxyl fatty acids, and combinations thereof; (iii) preparing a second reaction mixture comprising the hydroxyl fatty acids produced in step (ii) and a lipase; and (iv) incubating the second reaction mixture for a sufficient time to produce the musk lactone.

In the first reaction mixture, fatty acids are substrates that can be converted to a hydroxyl fatty acid by a cytochrome P450 hydroxylase in the presence of NADPH. In some embodiments, the one or more fatty acids in the first reaction mixture of step (i) comprise a linear fatty acid comprising at least 12 carbons (e.g., at least 12 carbons, at least 13 carbons, at least 14 carbons, at least 15 carbons, at least 16 carbons, at least 17 carbons, at least 18 carbons, at least 19 carbons, at least 20 carbons, or at least 25 carbons). In some embodiments, the one or more fatty acids in the first reaction mixture of step (i) comprise a linear fatty acid comprising 12-28 (e.g., 12-28, 12-25, 12-20, 12-15, 15-28, 15-25, 15-20, 20-28, 20-25, or 25-28) carbons. In some embodiments, the one or more fatty acids in the first reaction mixture of step (i) comprise a linear fatty acid comprising 12, 13, 14, 15, 16, 17, 18, 19, 20, 21, 22, 23, 24, 25, 26, 27, or 28 carbons, or any combinations thereof. In some embodiments, the one or more fatty acids in the first reaction mixture of step (i) comprise a linear fatty acid comprising 15, 16, 17, 18, 19, or 20 carbons, or any combinations thereof. In some embodiments, the one or more fatty acids in the first reaction mixture of step (i) comprises a mixture of linear fatty acid comprising different numbers of carbons, e.g., a mix of fatty acids comprising 15, 16, 17, 18, 19, or 20 carbons.

In some embodiments, the one or more fatty acids in the first reaction mixture of step (i) comprises a saturated fatty acid (i.e., no double bonds between any two carbons). In some embodiments, the one or more fatty acids in the first reaction mixture of step (i) comprises an unsaturated fatty acid. In some embodiments, the unsaturated fatty acid comprises at least one (e.g., 1, 2, 3, 4, 5, or more) double bond. In some embodiments, the unsaturated fatty acid comprises at least one (e.g., 1, 2, 3, 4, 5, or more) Z double bond.

In some embodiments, the one or more fatty acids in the first reaction mixture of step (i) are selected from the group consisting of:

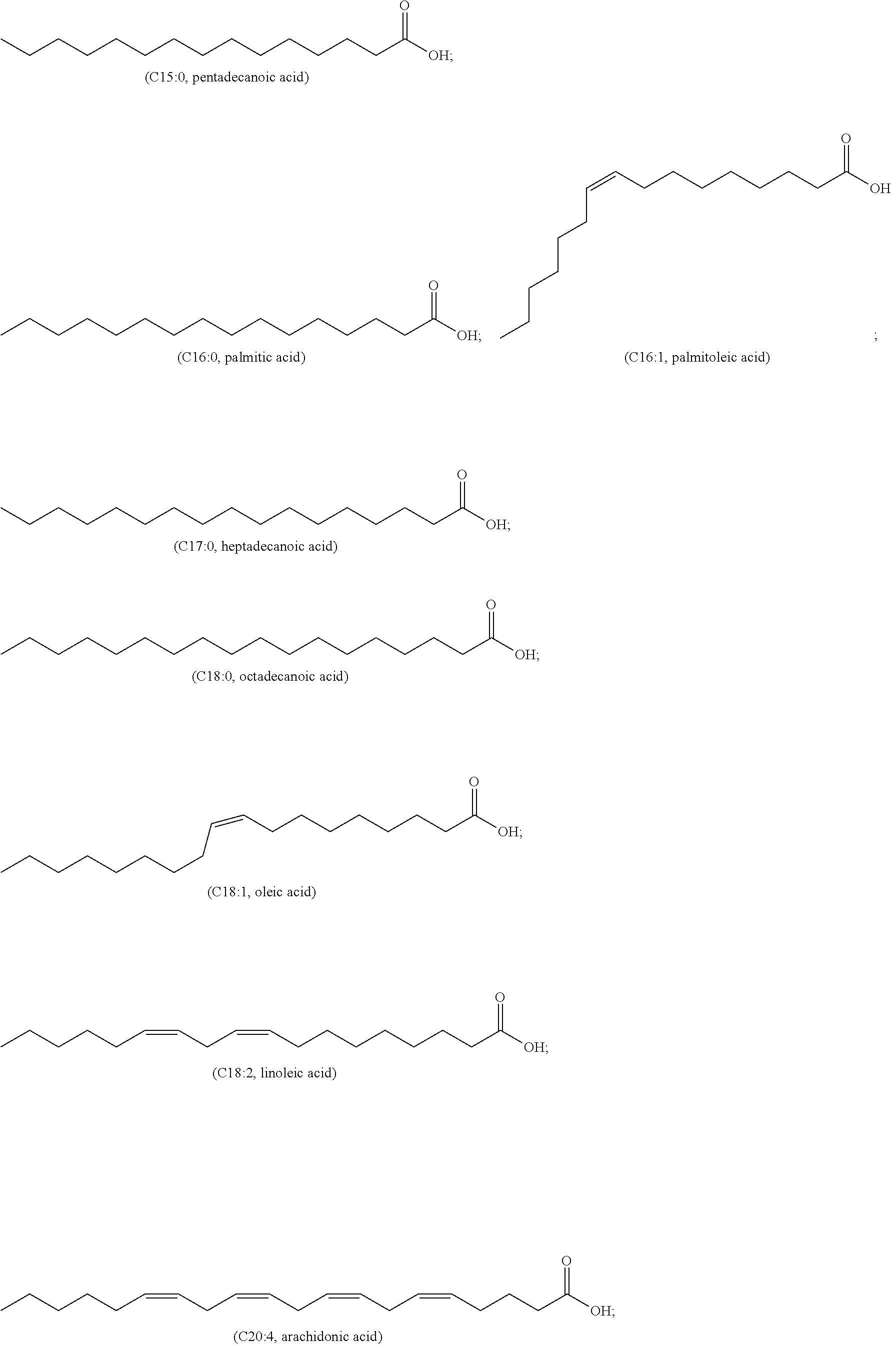

and any combinations thereof.

In some embodiments, the one or more fatty acids in the first reaction mixture of step (i) are selected from the group consisting of:

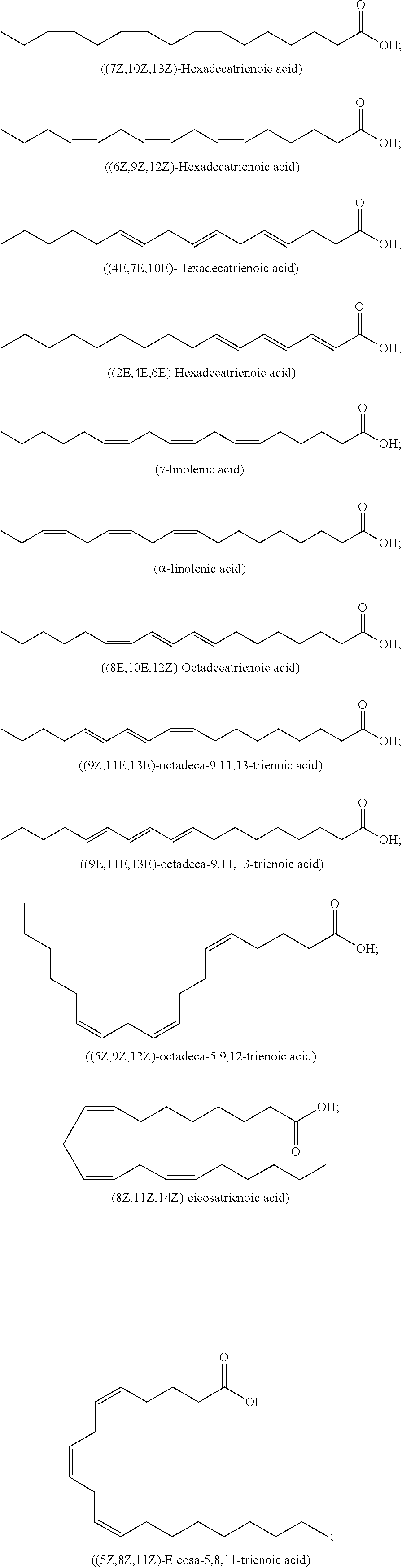

and any combinations thereof.

In some embodiments, the one or more fatty acids in the first reaction mixture of step (i) are selected from the group consisting of:

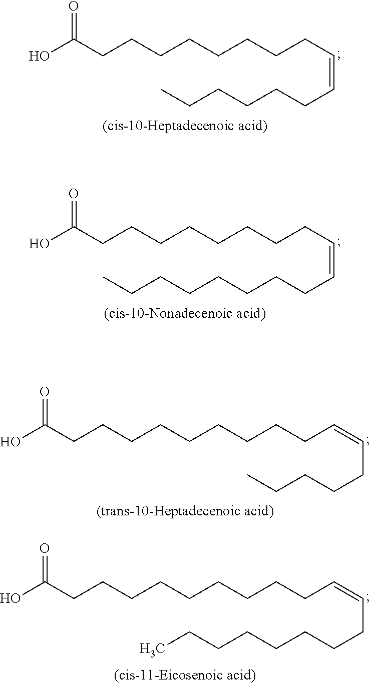 [SMILES: CCCCCC/C=C\CCCCCCCCC(=O)O ; CCCCCCCC/C=C\CCCCCCCCC(=O)O ; CCCCC/C=C\CCCCCCCCCC(=O)O ; CCCCCCCC/C=C\CCCCCCCCCC(=O)O]

and any combinations thereof.

The enzyme that hydroxylates the fatty acid substrates in the first reaction mixture is a cytochrome P450 enzyme that has hydroxylase activity (referred to herein as “cytochrome P450 hydroxylase”), or a functional variant thereof. In some embodiments, the cytochrome P450 hydroxylase is a bacterial cytochrome P450 enzyme. Examples of Cytochrome P450 enzymes that may be used in accordance with the present disclosure include, without limitation, CYP102A1 from Bacillus megaterium; CYP106A1 from Bacillus megaterium DSM319; CYP106A2 from B. megaterium ATCC13368; CYP109B1 from B. subtilis 168; CYP109E1 from B. megaterium DSM19; CYP154C5 from Nocardia farcinica IFM 10152; CYP260A1 and CYP260B1 from Sorangium cellulosum Soce56; CYP154C3 from Streptomyces griseus SGR1085; CYP154C8 from Streptomyces sp. W2233-SM and CYP219A from Novosphingobium aromaticivorans DSM12444 (saro0307), CYP105A1 from Streptomyces griseolus ATCC 11796, CYP107E1 from Micromonospora griseorubida, CYP107D1 and CYP127A3 from Mesorhizobium loti MAFF303099 (mlr5876) CYP110A1, CYP110C1, CYP110D1 and CYP110E1 from Nostoc sp. PCC7120; CYP200A1 from Bradyrhizobium japonicum USDA110; CYP102A15 and CYP102A170 from polar Bacillus sp. PAMC 25034 and Paenibacillus sp. PAMC 22724, respectively; CYP709C1 from the wheat plant (Triticum aestivum); CYP147G1 from Mycobacterium marinum; CYP505D6 from White-Rot Fungus Phanerochaete chrysosporium; and the CYP102 family enzymes (bamf2522 and bamf0695) from Bacillus amyloliquefaciens DSM 7.

It was shown herein that a cytochrome P450 enzyme from Bacillus megaterium (CYP102A1, e.g., as described in Miura et al., Biochim. Biophys. Acta. 388: 305-317, 1975, incorporated herein by reference) and a cytochrome P450 enzyme from Myceliophthora thermophile (CYP505A30, UniProt Accession No.: G2QDZ3) were able to carry out the first step of the biosynthetic method described herein, i.e., converting fatty acids to mono-hydroxyl fatty acids that are hydroxylated at one of ω-1, ω-2, or ω-3 position, or combinations of the mono-hydroxyl fatty acids thereof. The amino acid sequence of CYP102A1 is provided as SEQ ID NO: 1. The amino acid sequence of CYP505A30 is provided as SEQ ID NO: 3.

In some embodiments, the cytochrome P450 hydroxylase used in the biosynthetic methods described herein comprises an amino acid sequence that is at least 70% (e.g., at least 70%, at least 75%, at least 80%, at least 85%, at least 90%, at least 95%, or at least 99%) identical to the amino acid sequence of SEQ ID NO: 1. In some embodiments, the cytochrome P450 hydroxylase used in the biosynthetic methods described herein comprises an amino acid sequence that is 70%, 75%, 80%, 85%, 90%, 95%, or 99% identical to the amino acid sequence of SEQ ID NO: 1. In some embodiments, the cytochrome P450 hydroxylase used in the biosynthetic methods described herein comprises the amino acid sequence of SEQ ID NO: 1.

In some embodiments, the cytochrome P450 hydroxylase used in the biosynthetic methods described herein comprises an amino acid sequence that is at least 70% (e.g., at least 70%, at least 75%, at least 80%, at least 85%, at least 90%, at least 95%, or at least 99%) identical to the amino acid sequence of SEQ ID NO: 3. In some embodiments, the cytochrome P450 hydroxylase used in the biosynthetic methods described herein comprises an amino acid sequence that is 70%, 75%, 80%, 85%, 90%, 95%, or 99% identical to the amino acid sequence of SEQ ID NO: 3. In some embodiments, the cytochrome P450 hydroxylase used in the biosynthetic methods described herein comprises the amino acid sequence of SEQ ID NO: 3.

In some embodiments, the first reaction mixture of step (i) is an in vitro reaction mixture, e.g., using an isolated recombinantly produced cytochrome P450 hydroxylase. In some embodiments, the first reaction mixture of step (i) is a cell-based reaction mixture. In some embodiments, the cell-based reaction mixture comprises a cell selected from the group consisting of a yeast, a plant, an alga, a fungus, and a bacterium. In some embodiments, the cell-based reaction mixture comprises a bacterial cell of a genus selected from the group consisting of Escherichia; Salmonella; Bacillus; Acinetobacter; Corynebacterium; Methylosinus; Methylomonas; Rhodococcus; Pseudomonas; Rhodobacter; Synechocystis; Brevibacteria; Microbacterium; Arthrobacter; Citrobacter; Escherichia; Klebsiella; Pantoea; Salmonella; Corynebacterium; and Clostridium. In some embodiments, the cell-based reaction mixture comprises an E. coli cell. In some embodiments, the cell-based reaction mixture comprises a fungus of a genus selected from the group consisting of Saccharomyces; Zygosaccharomyces; Kluyveromyces; Candida; Streptomyces; Hansenula; Debaryomyces; Mucor; Pichia; Torulopsis; Aspergillus; and Arthrobotlys.

In some embodiments, the cell used in the cell-based reaction mixture recombinantly express the cytochrome P450 hydroxylase. For example, the cell used in the cell-based reaction mixture may be transformed with a nucleic acid molecule (e.g., a vector such as an expression vector) comprising a nucleotide sequence encoding the cytochrome P450 hydroxylase. In some embodiments, the nucleotide sequence encoding the cytochrome P450 hydroxylase is operably linked to a promoter (e.g., an inducible promoter or a constitutive promoter). The transformed cells can be cultured under conditions that allow the expression of the cytochrome P450 hydroxylase. The cells contain the expressed cytochrome P450 hydroxylase can be collected and used in the cell-based reaction mixture. Nucleotide sequences encoding CYP102A1 (SEQ ID NO: 1) is provided as SEQ ID NO: 2. Nucleotide sequences encoding CYP505A30 (SEQ ID NO: 3) is provided as SEQ ID NO: 4.

In some embodiments, the cell (e.g., a bacterial cell such as an E. coli or Bacillus cell) used in the cell-based reaction mixture is transformed with a nucleic acid molecule comprising a nucleotide sequence that is at least 70% (e.g., at least 70%, at least 75%, at least 80%, at least 85%, at least 90%, at least 95%, or at least 99%) to the nucleotide sequence of SEQ ID NO: 2 or SEQ ID NO: 4. In some embodiments, the cell (e.g., a bacterial cell such as an E. coli or Bacillus cell) used in the cell-based reaction mixture is transformed with a nucleic acid molecule comprising a nucleotide sequence that is 70%, 75%, 80%, 85%, 90%, 95%, or 99% to the nucleotide sequence of SEQ ID NO: 2 or SEQ ID NO: 4. In some embodiments, the cell (e.g., a bacterial cell such as an E. coli or Bacillus cell) used in the cell-based reaction mixture is transformed with a nucleic acid molecule comprising a nucleotide the nucleotide sequence of SEQ ID NO: 2 or SEQ ID NO: 4.

In some embodiments, in step (ii) of the biosynthetic methods described herein, the first reaction mixture is incubated for a sufficient time to produce hydroxyl fatty acids. In some embodiments, the first reaction mixture is incubated for at least 1 hour (e.g., at least 1 hour, at least 2 hours, at least 5 hours, at least 10 hours, or longer). In some embodiments, the incubation is under 37° C. In some embodiments, the hydroxyl fatty acids produced in step (ii) of the biosynthetic methods described herein are mono-hydroxyl fatty acids that is hydroxylated at any one of positions ω-1, ω-2, or ω-3. In some embodiments, the hydroxyl fatty acids produced in step (ii) of the biosynthetic methods described herein comprises ω-1 hydroxyl fatty acids, ω-2 hydroxyl fatty acid, ω-3 hydroxyl fatty acid, and combinations thereof, and any combinations thereof.

In some embodiments, step (ii) of the biosynthetic method described herein further comprises isolating the hydroxyl fatty acids from the reaction mixture. Any suitable extraction methods may be used. For example, the hydroxyl fatty acids may be extracted by liquid-liquid extraction using a mixture of hydrocarbon-based organic solvents (e.g., hexane) and a water-insoluble polar solvent (e.g., ethyl acetate).

The hydroxyl fatty acids produced in step (ii) can be further converted to lactones via macrolactonization catalyzed by a lipase. In some embodiments, as step (iii) of the biosynthetic methods described herein, a second reaction mixture is prepared, the second reaction mixture comprising the hydroxyl fatty acids produced in step (ii) and a lipase. One skilled in the art is able to identify suitable lipase for use in this reaction. In some embodiments, the lipase used in the second reaction mixture is lipase B from Candida antarctica (Uniprot Accession No.: P41365). The amino acid sequence of lipase B from Candida antarctica is provided as SEQ ID NO: 5. In this regard, Krishna et al. (Catalysis Reviews, Vol. 44, pp. 499-591, 2002) provides an overview of lipase-catalyzed esterifications in organic solvents. Each lipase can be tested with different solvents to identify the combination best suited to the lactonization of hydroxy fatty acids to yield musk lactones.

In some embodiments, the lipase used in the biosynthetic methods described herein comprises an amino acid sequence that is at least 70% (e.g., at least 70%, at least 75%, at least 80%, at least 85%, at least 90%, at least 95%, or at least 99%) identical to the amino acid sequence of SEQ ID NO: 5. In some embodiments, the lipase used in the biosynthetic methods described herein comprises an amino acid sequence that is 70%, 75%, 80%, 85%, 90%, 95%, or 99% identical to the amino acid sequence of SEQ ID NO: 5. In some embodiments, the lipase used in the biosynthetic methods described herein comprises the amino acid sequence of SEQ ID NO: 5.

In some embodiments, the lipase used in the second reaction mixture in step (iii) of the biosynthetic methods described herein is immobilized on a solid support (e.g., acrylic resin). In some embodiments, the lipase used in the second reaction mixture in step (iii) of the biosynthetic methods described herein is Novozyme 435 (lipase B from Candida antarctica immobilized on acrylic resin, available from Sigma, catalog #L4777). In some embodiments, wherein the second reaction mixture further comprises a solvent. Any suitable solvent may be used. In some embodiments, the solvent is toluene or dichloroethane.

In some embodiments, in the second reaction mixture, the hydroxyl fatty acids are at a total concentration of 0.02-0.1 M (e.g., 0.02 M, 0.03 M, 0.04 M, 0.05 M, 0.06 M, 0.07 M, 0.08 M, 0.09 M, or 0.1 M). In some embodiments, in the second reaction mixture, the hydroxyl fatty acids are at a total concentration of 0.025-0.05 M (e.g., 0.025 M, 0.03 M, 0.035 M, 0.04 M, 0.045 M, or 0.05 M). In some embodiments, in the second reaction mixture, the lipase is at a concentration of 20-150 g/L (e.g., 20-150 g/L, 20-100 g/L, 20-50 g/L, 50-150 g/L, 50-100 g/L, or 100-150 g/L). In some embodiments, in the second reaction mixture, the lipase is at a concentration of 50-100 g/L (e.g., 50 g/L, 60 g/L, 70 g/L, 80 g/L, 90 g/L, or 100 g/L).

In some embodiments, as step (iv) of the biosynthetic methods described herein, the second reaction mixture is incubated for a sufficient time to produce hydroxyl fatty acids. In some embodiments, the second reaction mixture is incubated for at least 10 hours (e.g., at least 10 hours, at least 15 hours, at least 20 hours, at least 24 hours, or longer). In some embodiments, the second reaction mixture is incubated for 15-24 hours (e.g., 15, 16, 17, 18, 19, 20, 21, 22, 23, or 24 hours). In some embodiments, the incubation is under 40-60° C. (e.g., 40-60° C., 40-55° C., 40-50° C., 40-45° C., 45-60° C., 45-55° C., 45-50° C., 50-60° C., 50-55° C., or 55-60° C.). In some embodiments, the incubation is under 40° C., 45° C., 50° C., 55° C., or 60° C.

In some embodiments, following the incubation of the second reaction mixture, step (iv) further comprises isolating the lactone compounds from the reaction mixture. Known methods of isolating lactone compounds from reaction mixture can be used, including without limitation, filtration and/or chromatography methods. In some embodiments, the isolated lactone compounds are subjected to drying.

In some embodiments, the lactone compounds produced by the biosynthetic methods described herein have a purity of at least 50% w/w (e.g., at least 50%, at least 60%, at least 70%, at least 75%, at least 80%, at least 85%, at least 90%, at least 95%, or at least 99%) w/w. The purity relates to all the lactone compounds produced, which, in some embodiments, comprise lactone compounds of different structures. In some embodiments, the lactone compounds produced in step (iv) has musk notes.

### Lactones

In some embodiments, the lactones (e.g., macrocyclic lactones) produced using the biosynthetic methods described herein comprise one or more compounds of the formula:

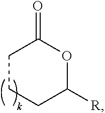

wherein:
   R is methyl, ethyl, or n-propyl;
   each [symbol] is independently a single bond, E double bond, Z double bond, or triple bond, as valency permits; and
   k is an integer between 6 and 30, inclusive.

In some embodiments, the lactones (e.g., macrocyclic lactones) produced using the biosynthetic methods described herein comprise one or more compounds of the formula:

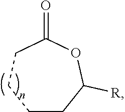

wherein:
   R is methyl, ethyl, or n-propyl;
   each [symbol] is independently a single bond, E double bond, Z double bond, or triple bond, as valency permits; and
   n is an integer between 6 and 20, inclusive.

In certain embodiments, R is methyl. In certain embodiments, R is ethyl. In certain embodiments, R is n-propyl.

In certain embodiments, each [symbol] is a single bond. In certain embodiments, at least one (e.g., one, two, three, or four) [symbol] is an E or Z double bond. In certain embodiments, at least one (e.g., one, two, three, or four) [symbol] is an E double bond. In certain embodiments, at least one (e.g., one, two, three, or four) [symbol] is an Z double bond. In certain embodiments, each [symbol] is independently a single bond, E double bond, or Z double bond. In certain embodiments, each [symbol] is independently a single or Z double bond. In certain embodiments, one [symbol] is a Z double bond, and each remaining [symbol] is a single bond. In certain embodiments, two [symbol] are Z double bonds, and each remaining [symbol] is a single bond. In certain embodiments, three [symbol] are Z double bonds, and each remaining [symbol] is a single bond. In certain embodiments, four [symbol] are Z double bonds, and each remaining [symbol] is a single bond. In certain embodiments, at least one (e.g., one, two, or three) [symbol] is a triple bond. In certain embodiments, at least one of any two adjacent [symbol] is a single bond.

In certain embodiments, one [symbol] is an E or Z double bond, and the remining [symbol] are single bonds. In certain embodiments, two [symbol] are independently E or Z double bonds, and the remining [symbol] are single bonds. In certain embodiments, three [symbol] are independently E or Z double bonds, and the remining [symbol] are single bonds. In certain embodiments, four [symbol] are independently E or Z double bonds, and the remining [symbol] are single bonds. In certain embodiments, each double bond if present is a Z double bond. In certain embodiments, each double bond if present is an E double bond. In certain embodiments, the lactone does not comprise any one of C═C═C, C═C≡C, and C≡C═C.

In certain embodiments, k is 6. In certain embodiments, k is 7. In certain embodiments, k is 8. In certain embodiments, k is 9. In certain embodiments, k is 10. In certain embodiments, k is 11. In certain embodiments, k is 12. In certain embodiments, k is 13. In certain embodiments, k is 14. In certain embodiments, k is 15. In certain embodiments, k is 16. In certain embodiments, k is 8, 9, 10, 11, 12, 13, or 15. In certain embodiments, k is 8, 9, 10, 11, 12, 13, 14, or 15. In certain embodiments, k is an integer between 17 and 20, inclusive. In certain embodiments, k is an integer between 21 and 25, inclusive. In certain embodiments, k is an integer between 26 and 30, inclusive.

In certain embodiments, n is 6. In certain embodiments, n is 7. In certain embodiments, n is 8. In certain embodiments, n is 9. In certain embodiments, n is 10. In certain embodiments, n is 11. In certain embodiments, n is 12. In certain embodiments, n is 13. In certain embodiments, n is 14. In certain embodiments, n is 15, 16, 17, 18, 19, or 20. In certain embodiments, n is an integer between 7 and 14, inclusive.

In certain embodiments, the lactones produced using the biosynthetic methods described herein include a chiral carbon atom, which is the carbon atom labelled with * in the formula:

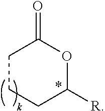

In certain embodiments, the lactones produced using the biosynthetic methods described herein include a chiral carbon atom, which is the carbon atom labelled with * in the formula:

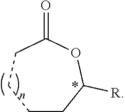

In certain embodiments, the chiral carbon atom is of the S configuration. In certain embodiments, the chiral carbon atom is of the R configuration.

In certain embodiments, the lactones produced using the biosynthetic methods described herein are a mixture of lactones. In certain embodiments, the lactones produced using the biosynthetic methods described herein are a mixture of (e.g., two or three) lactones having different n values and different R moieties when the difference in the R/S configurations if present is disregarded. In certain embodiments, the lactones produced using the biosynthetic methods described herein are a mixture of lactones having the same R/S configuration but different n values and different R moieties. In certain embodiments, the lactones produced using the biosynthetic methods described herein are a mixture of lactones having different R/S configurations but the same n values and the same R moieties. In certain embodiments, the lactones produced using the biosynthetic methods described herein are a mixture of lactones having different R/S configurations, different n values, and different R moieties. In certain embodiments, the lactones produced using the biosynthetic methods described herein are substantially (e.g., between 90% and 95%, between 95% and 97%, between 97% and 99%, or between 99% and 99.9%, inclusive, by mole) a racemic mixture of lactones.

In certain embodiments, the lactones produced using the biosynthetic methods described herein are substantially (e.g., between 90% and 95%, between 95% and 97%, between 97% and 99%, or between 99% and 99.9%, inclusive, by mole) one single type of lactone (e.g., substantially free of other types of lactones, including the opposite enantiomer of the one single type of lactone). In certain embodiments, the lactones produced using the biosynthetic methods described herein are substantially (between 90% and 95%, between 95% and 97%, between 97% and 99%, or between 99% and 99.9%, inclusive, by mole) two types of lactones, which are opposite enantiomers of each other (e.g., substantially free of other types of lactones).

In some embodiments, the one or more fatty acids used as substrates in the first reaction mixture in step (i) comprise

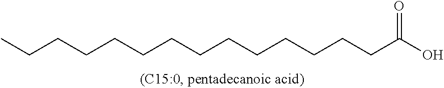 [SMILES: CCCCCCCCCCCCCCC(=O)O]

and the lactone produced in step (iv) comprises:

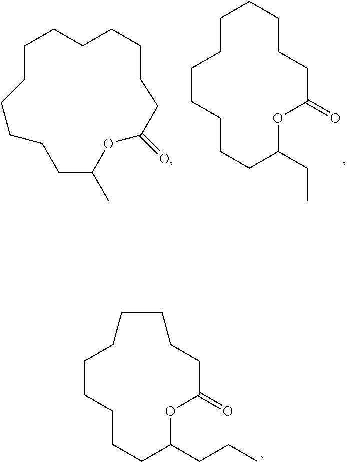

and combinations thereof.

In some embodiments, the one or more fatty acids used as substrates in the first reaction mixture in step (i) comprise

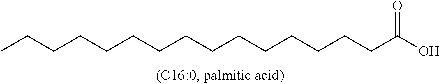 [SMILES: CCCCCCCCCCCCCCCC(=O)O]

and the lactone produced in step (iv) comprises:

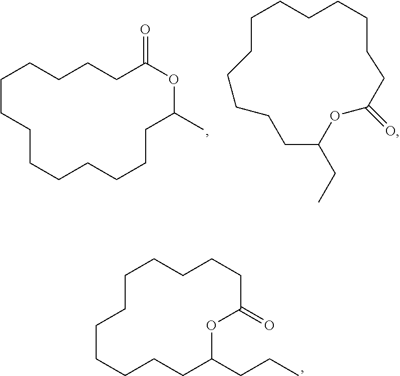

and combinations thereof.

In some embodiments, the one or more fatty acids used as substrates in the first reaction mixture in step (i) comprise

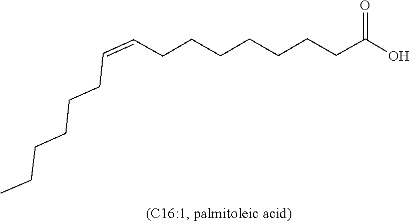 [SMILES: CCCCCC/C=C\CCCCCCCC(=O)O]

and the lactone produced in step (iv) comprises:

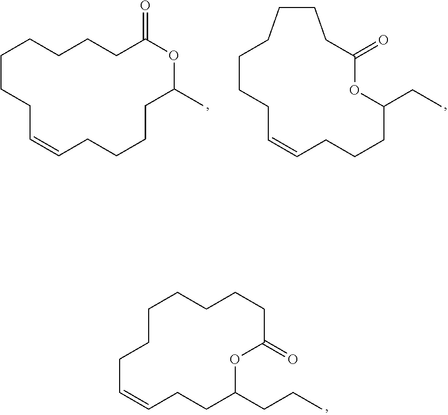

and combinations thereof.

In some embodiments, the one or more fatty acids used as substrates in the first reaction mixture in step (i) comprise

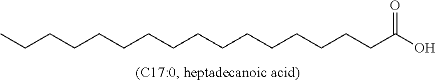 [SMILES: CCCCCCCCCCCCCCCCC(=O)O]

and the lactone produced in step (iv) comprises:

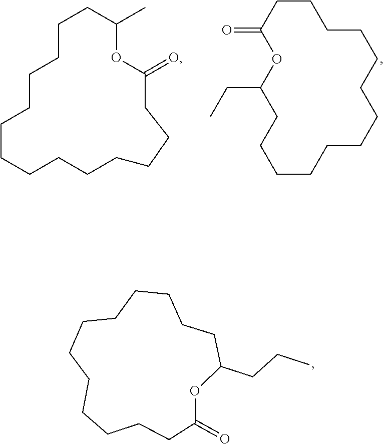

and combinations thereof.

In some embodiments, the one or more fatty acids used as substrates in the first reaction mixture in step (i) comprise

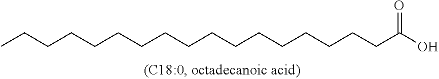 [SMILES: CCCCCCCCCCCCCCCCCC(=O)O]

and the lactone produced in step (iv) comprises:

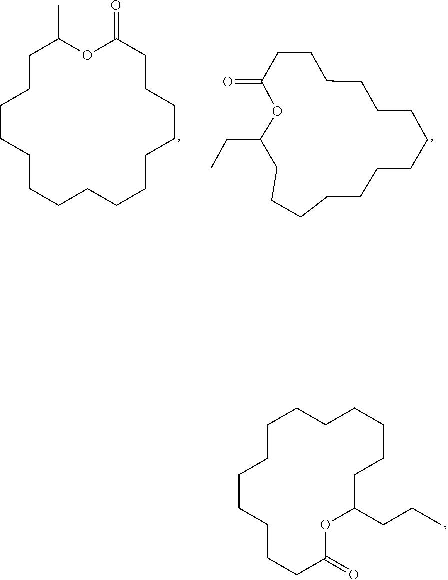 [SMILES: CC1CCCCCCCCCCCCCCCC(=O)O1 ; CCC1CCCCCCCCCCCCCCC(=O)O1 ; CCCC1CCCCCCCCCCCCCC(=O)O1]

and combinations thereof.

In some embodiments, the one or more fatty acids used as substrates in the first reaction mixture in step (i) comprise

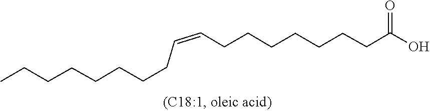 [SMILES: CCCCCCCC/C=C\CCCCCCCC(=O)O]

and the lactone produced in step (iv) comprises:

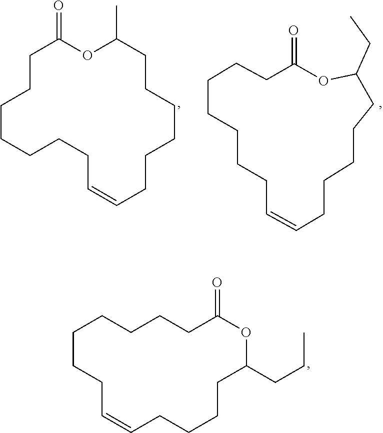

and combinations thereof.

In some embodiments, the one or more fatty acids used as substrates in the first reaction mixture in step (i) comprise

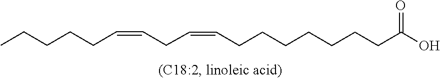 [SMILES: CCCCC/C=C\C/C=C\CCCCCCCC(=O)O]

and the lactone produced in step (iv) comprises:

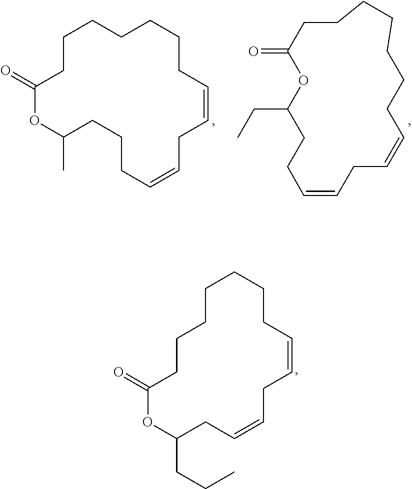

and combinations thereof.

In some embodiments, the one or more fatty acids used as substrates in the first reaction mixture in step (i) comprise

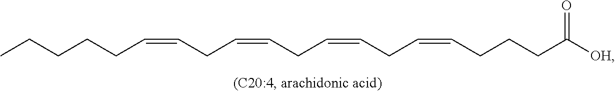 [SMILES: CCCCC/C=C\C/C=C\C/C=C\C/C=C\CCCC(=O)O]

and the lactone produced in step (iv) comprises:

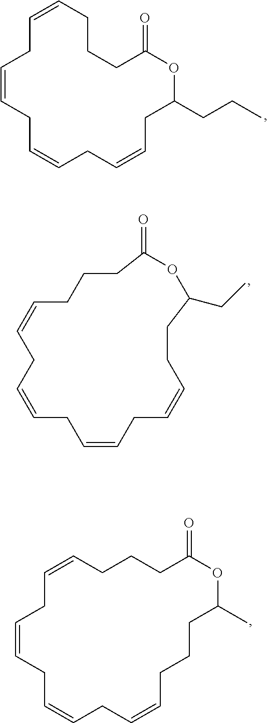 [SMILES: CCCC1C/C=C\C/C=C\C/C=C\C/C=C\CCCC(=O)O1 ; CCC1CC/C=C\C/C=C\C/C=C\C/C=C\CCCC(=O)O1 ; CC1CCC/C=C\C/C=C\C/C=C\C/C=C\CCCC(=O)O1]

and combinations thereof.

In some embodiments, the one or more fatty acids used as substrates in the first reaction mixture in step (i) comprise

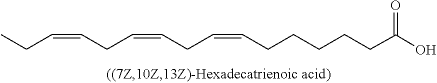 [SMILES: CC/C=C\C/C=C\C/C=C\CCCCCC(=O)O]

and the lactone produced in step (iv) comprises:

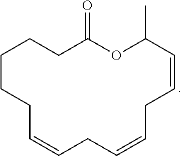 [SMILES: CC1/C=C\C/C=C\C/C=C\CCCCCC(=O)O1]

In some embodiments, the one or more fatty acids used as substrates in the first reaction mixture in step (i) comprise

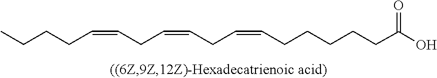 [SMILES: CCCC/C=C\C/C=C\C/C=C\CCCCCC(=O)O]

and the lactone produced in step (iv) comprises:

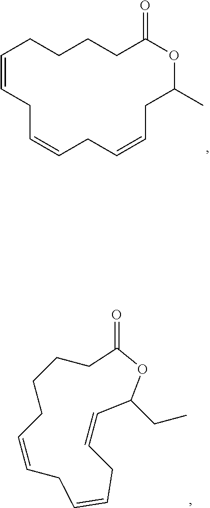 [SMILES: CC1C/C=C\C/C=C\C/C=C\CCCCC(=O)O1 ; CCC1/C=C\C/C=C\C/C=C\CCCCC(=O)O1]

and combinations thereof.

In some embodiments, the one or more fatty acids used as substrates in the first reaction mixture in step (i) comprise

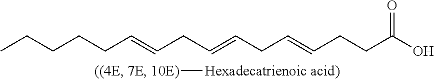

and the lactone produced in step (iv) comprises:

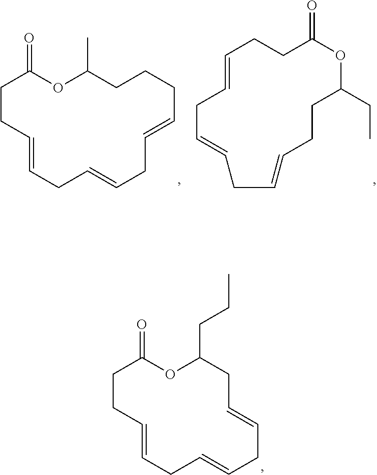

and combinations thereof.

In some embodiments, the one or more fatty acids used as substrates in the first reaction mixture in step (i) comprise

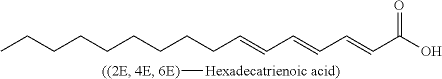

and the lactone produced in step (iv) comprises:

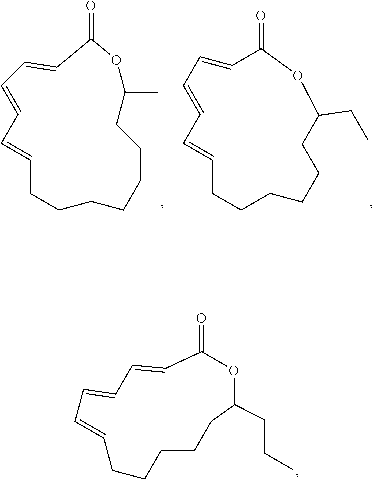

and combinations thereof.

In some embodiments, the one or more fatty acids used as substrates in the first reaction mixture in step (i) comprise

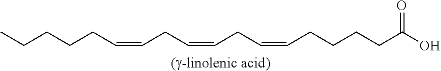 [SMILES: CCCCC/C=C\C/C=C\C/C=C\CCCCC(=O)O]

and the lactone produced in step (iv) comprises:

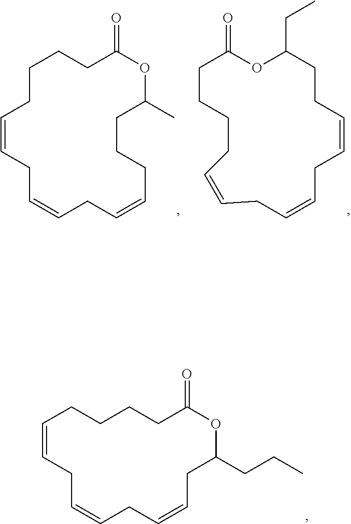

and combinations thereof.

In some embodiments, the one or more fatty acids used as substrates in the first reaction mixture in step (i) comprise

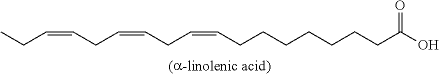 [SMILES: CC/C=C\C/C=C\C/C=C\CCCCCCCC(=O)O]

and the lactone produced in step (iv) comprises:

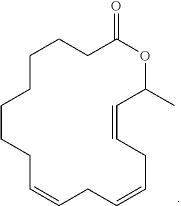 [SMILES: CC1/C=C\C/C=C\C/C=C\CCCCCCCC(=O)O1]

In some embodiments, the one or more fatty acids used as substrates in the first reaction mixture in step (i) comprise

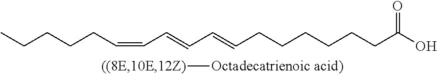

and the lactone produced in step (iv) comprises:

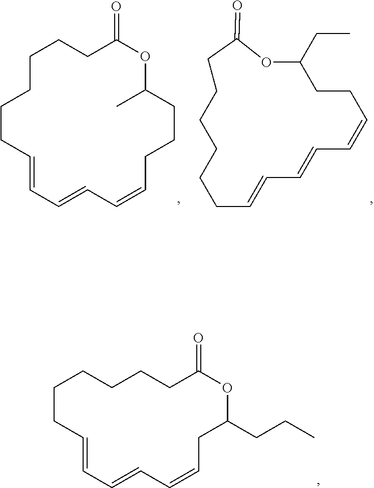

and combinations thereof.

In some embodiments, the one or more fatty acids used as substrates in the first reaction mixture in step (i) comprise

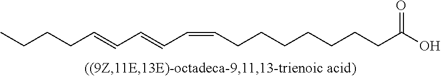 [SMILES: CCCC/C=C/C=C/C=C\CCCCCCCC(=O)O]

and the lactone produced in step (iv) comprises:

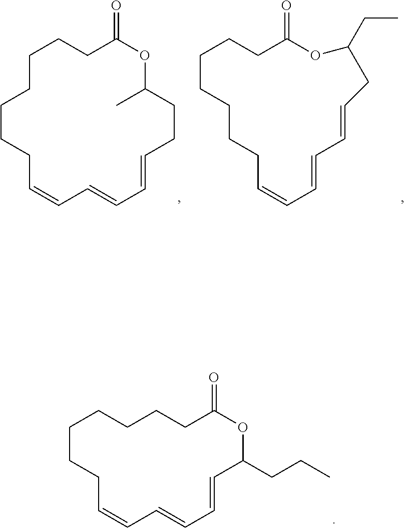

and combinations thereof.

In some embodiments, the one or more fatty acids used as substrates in the first reaction mixture in step (i) comprise

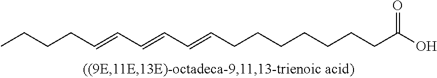 [SMILES: CCCC/C=C/C=C/C=C/CCCCCCCC(=O)O]

and the lactone produced in step (iv) comprises:

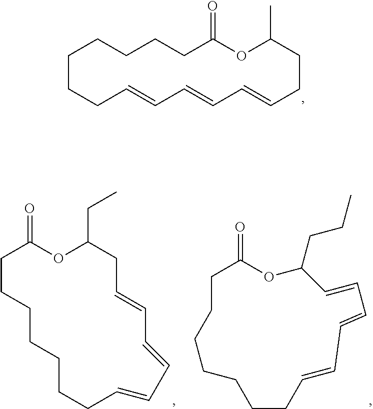

and combinations thereof.

In some embodiments, the one or more fatty acids used as substrates in the first reaction mixture in step (i) comprise

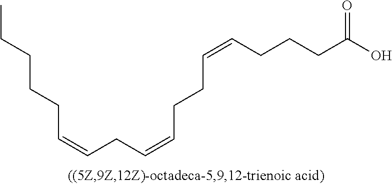 [SMILES: CCCCC/C=C\C/C=C\CC/C=C\CCCC(=O)O]

and the lactone produced in step (iv) comprises:

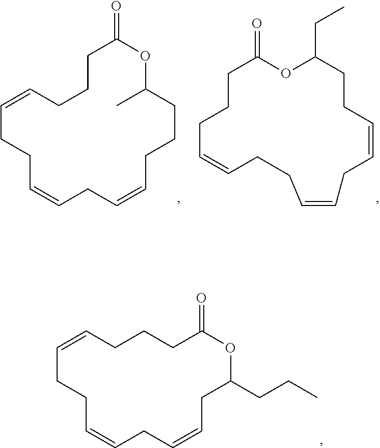

and combinations thereof.

In some embodiments, the one or more fatty acids used as substrates in the first reaction mixture in step (i) comprise

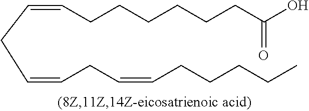 [SMILES: CCCCC/C=C\C/C=C\C/C=C\CCCCCCC(=O)O]

and the lactone produced in step (iv) comprises:

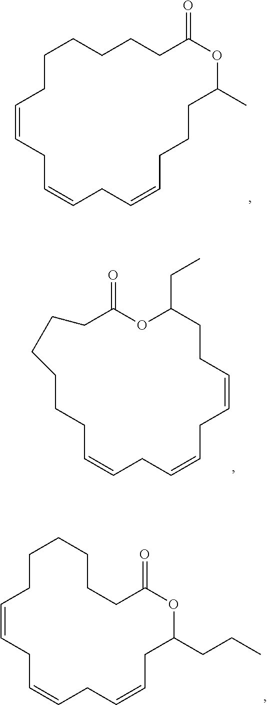 [SMILES: CC1CCC/C=C\C/C=C\C/C=C\CCCCCCC(=O)O1 ; CCC1CC/C=C\C/C=C\C/C=C\CCCCCCC(=O)O1 ; CCCC1C/C=C\C/C=C\C/C=C\CCCCCCC(=O)O1]

and combinations thereof.

In some embodiments, the one or more fatty acids used as substrates in the first reaction mixture in step (i) comprise

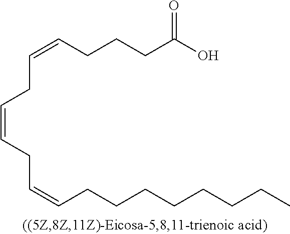 [SMILES: CCCCCCCC/C=C\C/C=C\C/C=C\CCCC(=O)O]

and the lactone produced in step (iv) comprises:

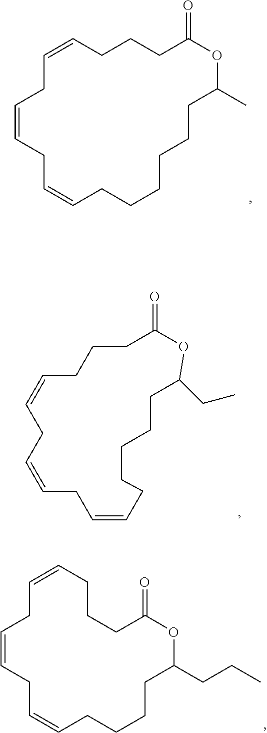 [SMILES: CC1CCCCCC/C=C\C/C=C\C/C=C\CCCC(=O)O1 ; CCC1CCCCC/C=C\C/C=C\C/C=C\CCCC(=O)O1 ; CCCC1CCCC/C=C\C/C=C\C/C=C\CCCC(=O)O1]

and combinations thereof.

In some embodiments, the one or more fatty acids used as substrates in the first reaction mixture in step (i) comprise

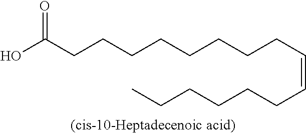 [SMILES: CCCCCC/C=C\CCCCCCCCC(=O)O]

and the lactone produced in step (iv) comprises:

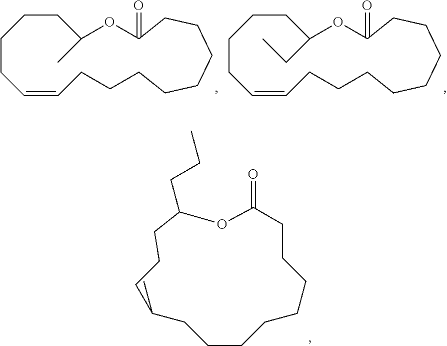

and combinations thereof.

In some embodiments, the one or more fatty acids used as substrates in the first reaction mixture in step (i) comprise

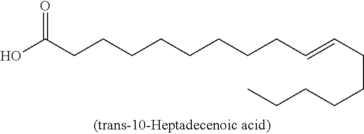 [SMILES: CCCCCC/C=C/CCCCCCCCC(=O)O]

and the lactone produced in step (iv) comprises:

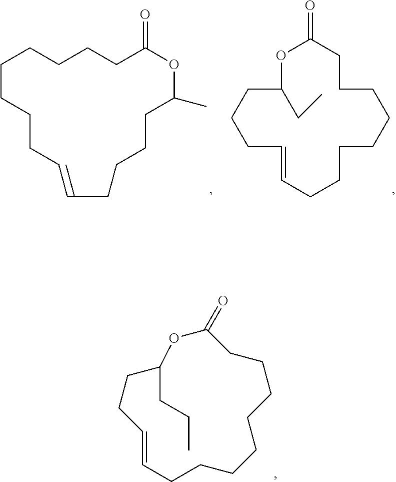

and combinations thereof.

In some embodiments, the one or more fatty acids used as substrates in the first reaction mixture in step (i) comprise

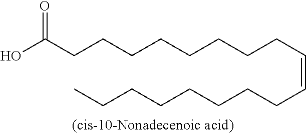 [SMILES: CCCCCCCC/C=C\CCCCCCCCC(=O)O]

and the lactone produced in step (iv) comprises:

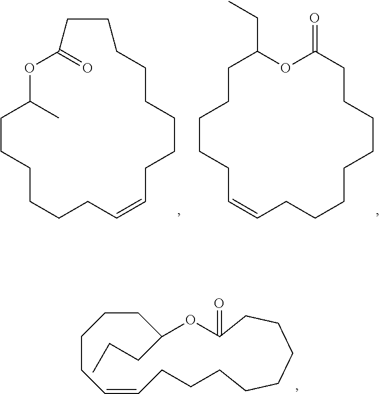

and combinations thereof.

In some embodiments, the one or more fatty acids used as substrates in the first reaction mixture in step (i) comprise

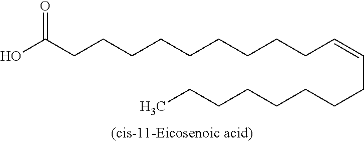 [SMILES: CCCCCCCC/C=C\CCCCCCCCCC(=O)O]

and the lactone produced in step (iv) comprises:

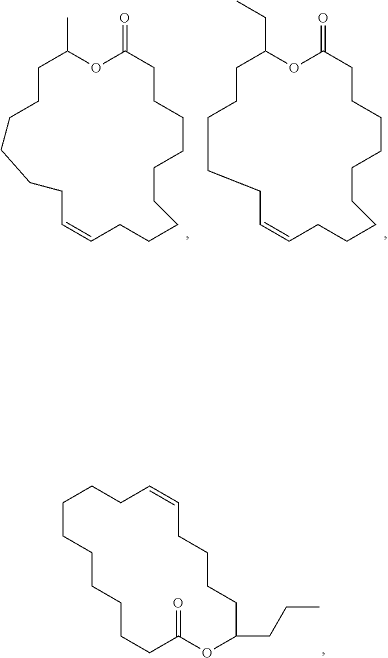

and combinations thereof. In some embodiments, the lactone produced using the biosynthetic methods provided herein comprises any one or more (e.g., 1, 2, 3, 4, 5, 6, 7, 8, 9, 10, 11, 12, 13, 14, 15, 16, 17, 18, 19, 20, 21, 22, 23, or 24) of the lactones provided in Table 1. In some embodiments, the lactone produced using the biosynthetic methods provided herein comprises any one or more (e.g., 25, 26, 27, 28, 29, 30, or 31) of the lactones provided in Table 2.

Other aspects of the present disclosure provide novel lactones of the formula:

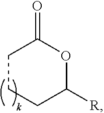

wherein:
   R is methyl, ethyl, or n-propyl;
   each [symbol] is independently a single bond, E double bond, Z double bond, or triple bond, as valency permits; and
   k is an integer between 6 and 30, inclusive;
   provided that the lactone is not of the formula:

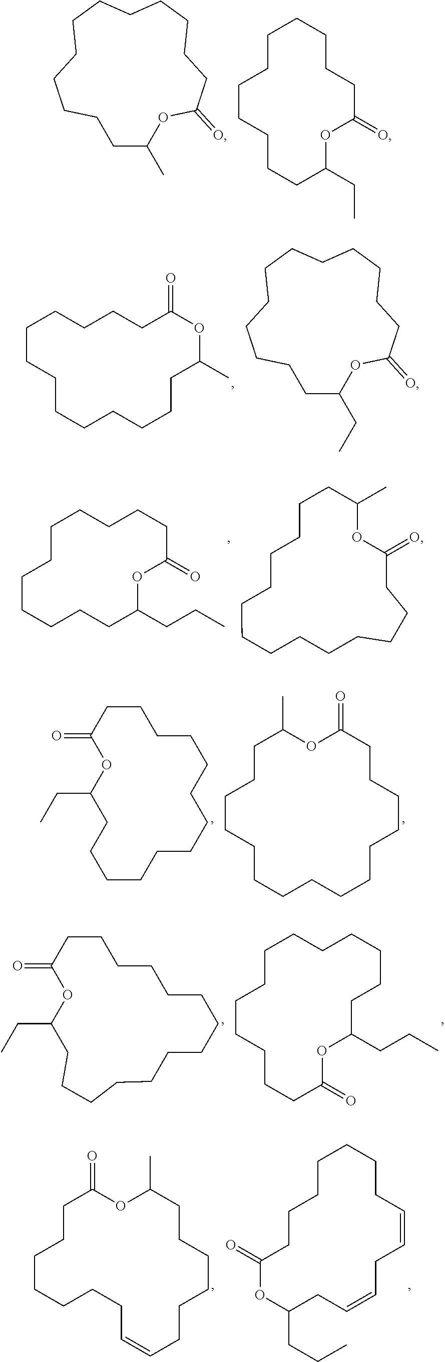

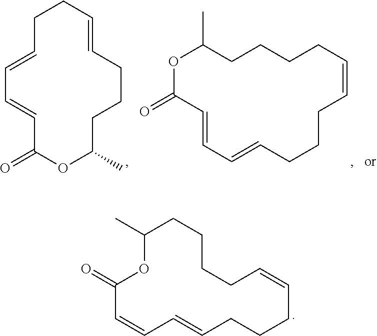

Other aspects of the present disclosure provide novel lactones of the formula:

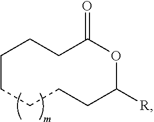

wherein:
   R is methyl, ethyl, or n-propyl;
   each [symbol] is independently a single bond or Z double bond, as valency permits, wherein 0, 1, 2, or 4 [symbol] are Z double bonds; and
   m is an integer between 4 and 11, inclusive;
   provided that the lactone is not of the formula:

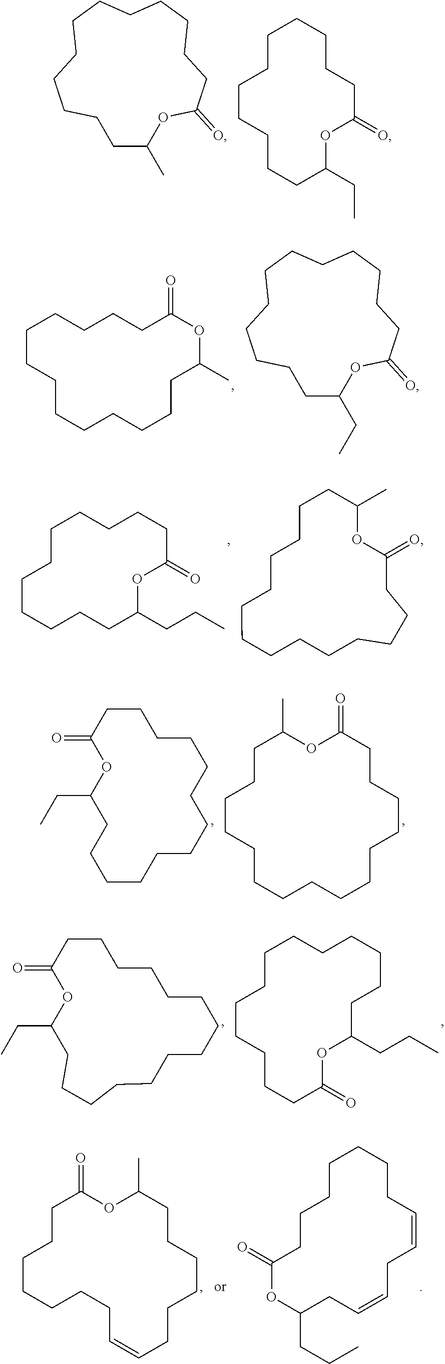

In certain embodiments, the novel lactone is of the formula:

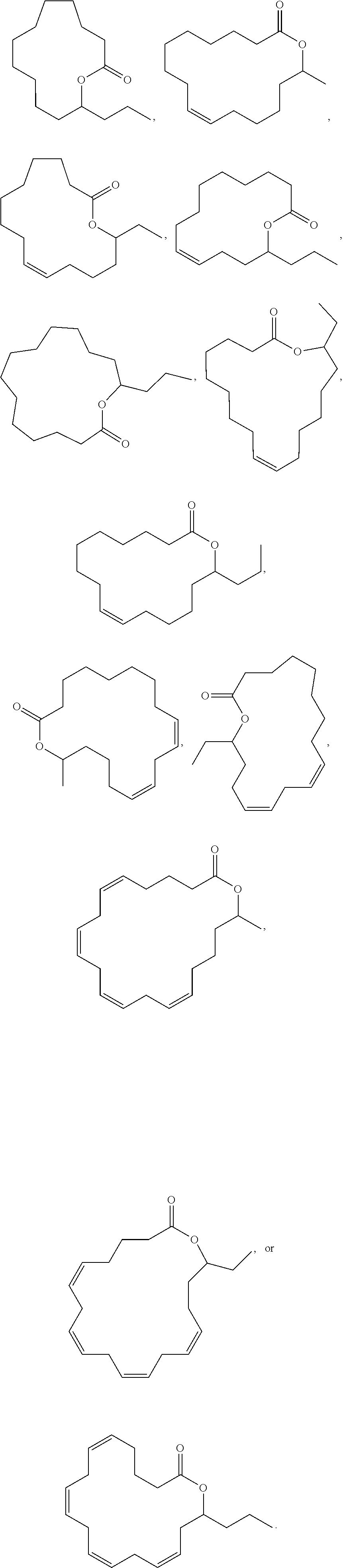

In certain embodiments, the novel lactone is of the formula:

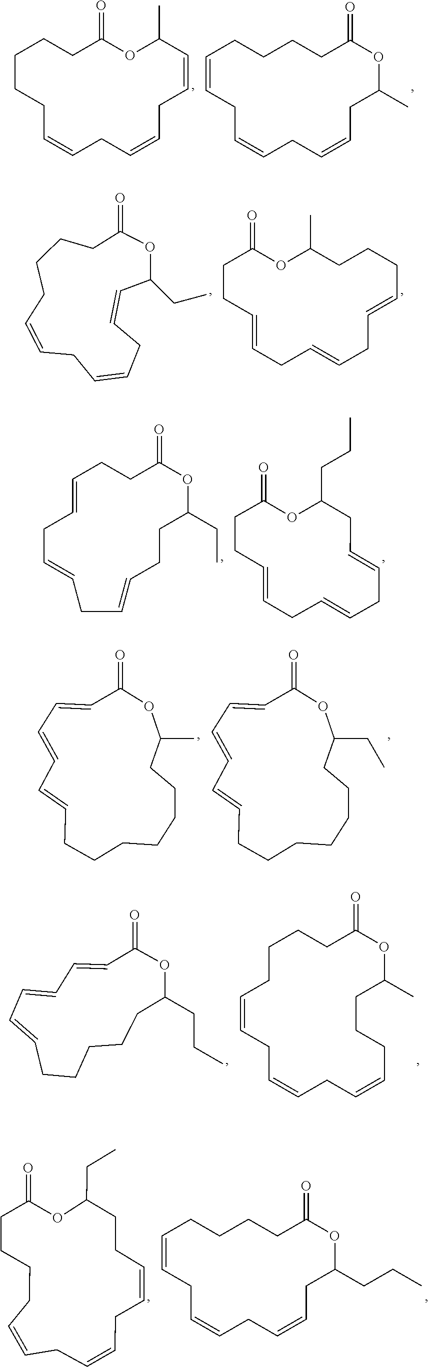

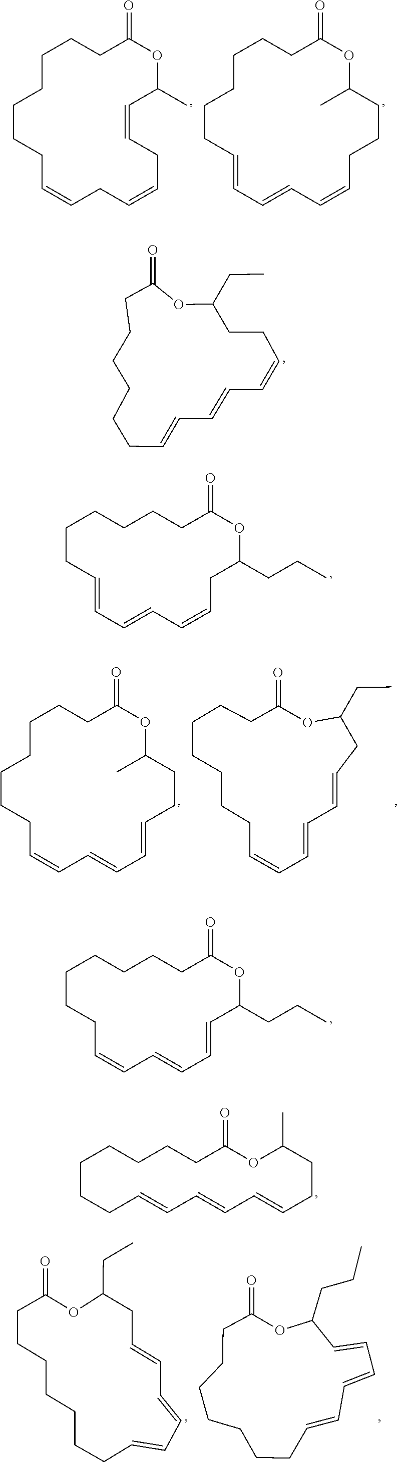

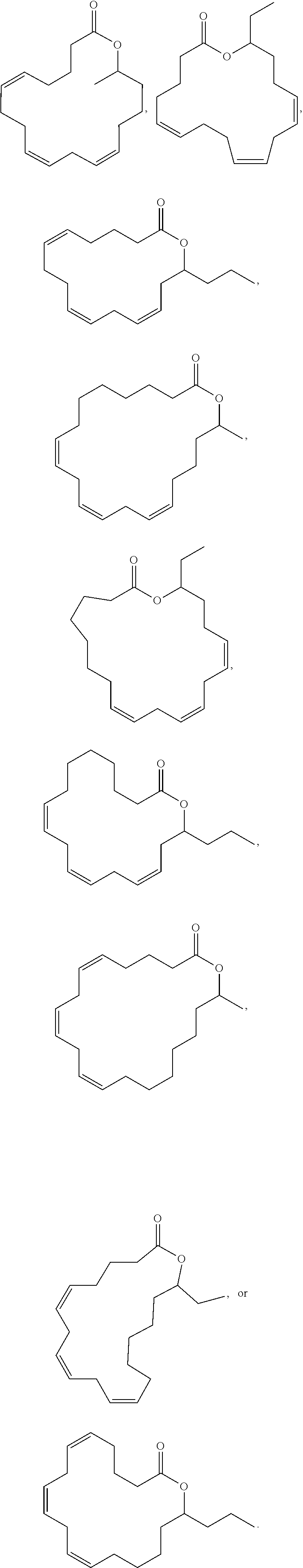

In certain embodiments, the novel lactone is of the formula:

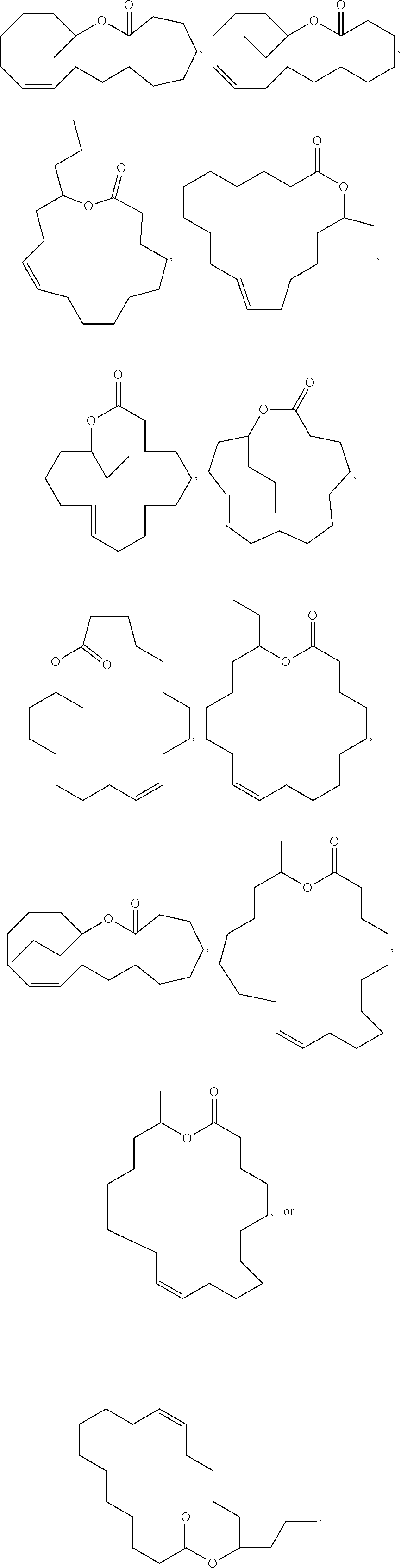

In certain embodiments, each [symbol] is a single bond. In certain embodiments, one [symbol] is a Z double bond, and the remining [symbol] are single bonds. In certain embodiments, two [symbol] are Z double bonds, and the remining [symbol] are single bonds. In certain embodiments, four [symbol] are Z double bonds, and the remining [symbol] are single bonds.

In certain embodiments, one [symbol] is an E or Z double bond, and the remining are single bonds. In certain embodiments, two [symbol] are independently E or Z double bonds, and the remining [symbol] are single bonds. In certain embodiments, three [symbol] are independently E or Z double bonds, and the remining [symbol] are single bonds. In certain embodiments, four [symbol] are independently E or Z double bonds, and the remining [symbol] are single bonds. In certain embodiments, each double bond if present is a Z double bond. In certain embodiments, each double bond if present is an E double bond. In certain embodiments, the lactone does not comprise any one of C═C═C, C═C≡C, and C≡C═C.

The variable k is as described herein.

In certain embodiments, m is 4. In certain embodiments, m is 5. In certain embodiments, m is 6. In certain embodiments, m is 7. In certain embodiments, m is 8. In certain embodiments, m is 9. In certain embodiments, m is 10. In certain embodiments, m is 11.

In certain embodiments, the chiral carbon atom of the novel lactone is of the S configuration. In certain embodiments, the chiral carbon atom of the novel lactone is of the R configuration.

Another aspect of the present disclosure provides a mixture of two or more novel lactones. In certain embodiments, the mixture of two or more novel lactones is a mixture of the opposite enantiomers of

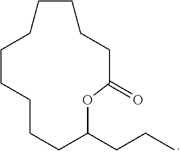 [SMILES: CCCC1CCCCCCCCCCC(=O)O1]

In certain embodiments, the mixture of two or more novel lactones is a mixture of the opposite enantiomers of

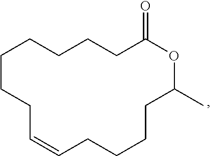 [SMILES: CC1CCCC/C=C\CCCCCCCC(=O)O1]

the opposite enantiomers of

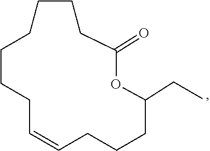 [SMILES: CCC1CCC/C=C\CCCCCCCC(=O)O1]

and the opposite enantiomers of

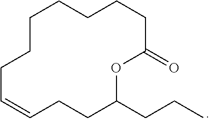 [SMILES: CCCC1CC/C=C\CCCCCCCC(=O)O1]

In certain embodiments, the mixture of two or more novel lactones is a mixture of the opposite enantiomers of

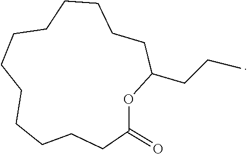 [SMILES: CCCC1CCCCCCCCCCCCC(=O)O1]

In certain embodiments, the mixture of two or more novel lactones is a mixture of the opposite enantiomers of

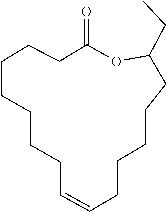 [SMILES: CCC1CCCCC/C=C\CCCCCCCC(=O)O1]

and the opposite enantiomers of

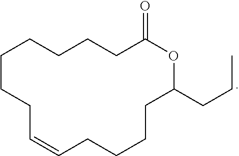 [SMILES: CCCC1CCCC/C=C\CCCCCCCC(=O)O1]

In certain embodiments, the mixture of two or more novel lactones is a mixture of the opposite enantiomers of

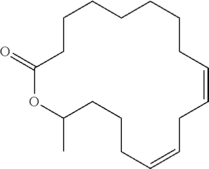 [SMILES: CC1CCC/C=C\C/C=C\CCCCCCCC(=O)O1]

and the opposite enantiomers of

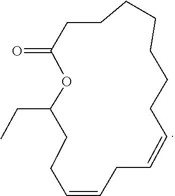 [SMILES: CCC1CC/C=C\C/C=C\CCCCCCCC(=O)O1]

In certain embodiments, the mixture of two or more novel lactones is a mixture of the opposite enantiomers of

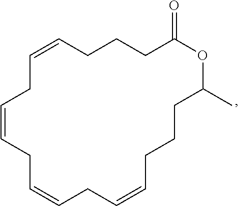 [SMILES: CC1CCC/C=C\C/C=C\C/C=C\C/C=C\CCCC(=O)O1]

the opposite enantiomers of

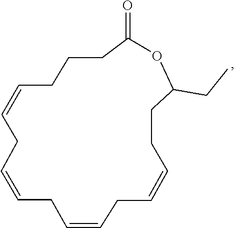 [SMILES: CCC1CC/C=C\C/C=C\C/C=C\C/C=C\CCCC(=O)O1]

and the opposite enantiomers of

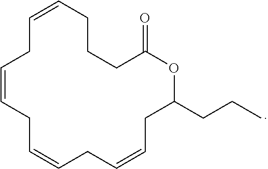 [SMILES: CCCC1C/C=C\C/C=C\C/C=C\C/C=C\CCCC(=O)O1]

In certain embodiments, the mixture of two or more novel lactones is a mixture of the opposite enantiomers of

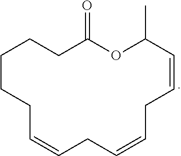 [SMILES: CC1/C=C\C/C=C\C/C=C\CCCCCC(=O)O1]

In certain embodiments, the mixture of two or more novel lactones is a mixture of the opposite enantiomers of

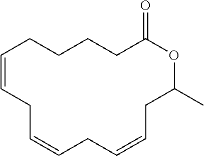 [SMILES: CC1C/C=C\C/C=C\C/C=C\CCCCC(=O)O1]

and the opposite enantiomers of

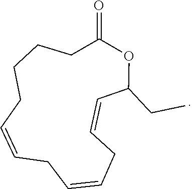 [SMILES: CCC1/C=C\C/C=C\C/C=C\CCCCC(=O)O1]

In certain embodiments, the mixture of two or more novel lactones is a mixture of the opposite enantiomers of

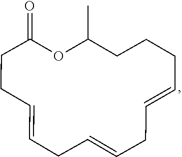 [SMILES: CC1CCC/C=C/C/C=C/C/C=C/CCC(=O)O1]

the opposite enantiomers of

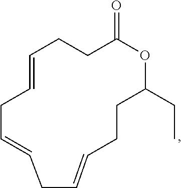 [SMILES: CCC1CC/C=C/C/C=C/C/C=C/CCC(=O)O1]

and the opposite enantiomers of

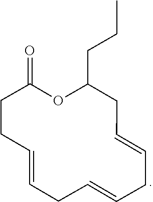 [SMILES: CCCC1C/C=C/C/C=C/C/C=C/CCC(=O)O1]

In certain embodiments, the mixture of two or more novel lactones is a mixture of the opposite enantiomers of

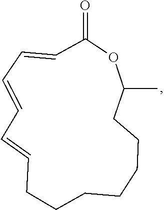 [SMILES: CC1CCCCCCC/C=C/C=C/C=C/C(=O)O1]

the opposite enantiomers of

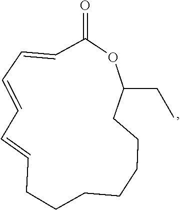 [SMILES: CCC1CCCCCCC/C=C/C=C/C=C/C(=O)O1]

and the opposite enantiomers of

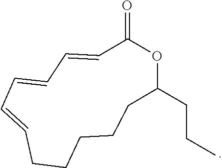 [SMILES: CCCC1CCCCC/C=C/C=C/C=C/C(=O)O1]

In certain embodiments, the mixture of two or more novel lactones is a mixture of the opposite enantiomers

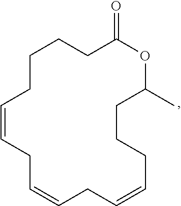 [SMILES: CC1CCC/C=C\C/C=C\C/C=C\CCCCC(=O)O1]

the opposite enantiomers of

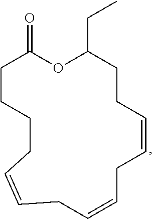 [SMILES: CCC1CC/C=C\C/C=C\C/C=C\CCCCC(=O)O1]

and the opposite enantiomers of

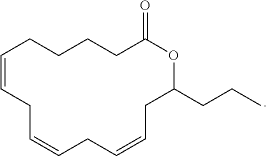 [SMILES: CCCC1C/C=C\C/C=C\C/C=C\CCCCC(=O)O1]

In certain embodiments, the mixture of two or more novel lactones is a mixture of the opposite enantiomers of

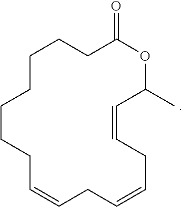 [SMILES: CC1/C=C\C/C=C\C/C=C\CCCCCCCC(=O)O1]

In certain embodiments, the mixture of two or more novel lactones is a mixture of the opposite enantiomers of

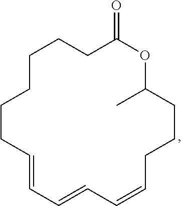 [SMILES: CC1CCC\C=C/C=C/C=C/CCCCCCC(=O)O1]

the opposite enantiomers of

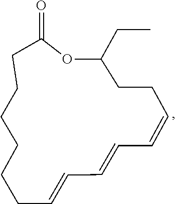 [SMILES: CCC1CC\C=C/C=C/C=C/CCCCCCC(=O)O1]

and the opposite enantiomers of

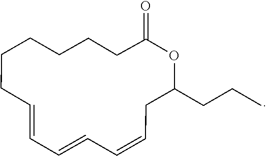 [SMILES: CCCC1C\C=C/C=C/C=C/CCCCCCC(=O)O1]

In certain embodiments, the mixture of two or more novel lactones is a mixture of the opposite enantiomers of

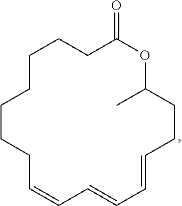 [SMILES: CC1CC/C=C/C=C/C=C\CCCCCCCC(=O)O1]

the opposite enantiomers of

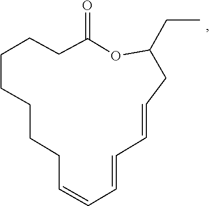 [SMILES: CCC1C/C=C/C=C/C=C\CCCCCCCC(=O)O1]

and the opposite enantiomers of

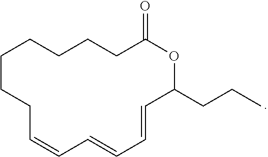 [SMILES: CCCC1/C=C/C=C/C=C\CCCCCCCC(=O)O1]

In certain embodiments, the mixture of two or more novel lactones is a mixture of the opposite enantiomers of

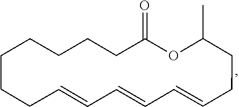 [SMILES: CC1CC/C=C/C=C/C=C/CCCCCCCC(=O)O1]

the opposite enantiomers of

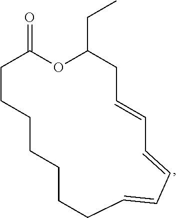 [SMILES: CCC1C/C=C/C=C/C=C/CCCCCCCC(=O)O1]

and the opposite enantiomers of

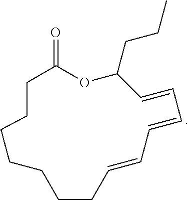 [SMILES: CCCC1/C=C/C=C/C=C/CCCCCCCC(=O)O1]

In certain embodiments, the mixture of two or more novel lactones is a mixture of the opposite enantiomers of

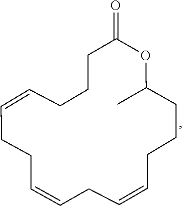 [SMILES: CC1CCC/C=C\C/C=C\CC/C=C\CCCC(=O)O1]

the opposite enantiomers of

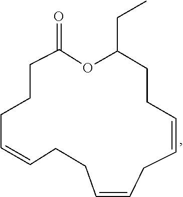 [SMILES: CCC1CC/C=C\C/C=C\CC/C=C\CCCC(=O)O1]

and the opposite enantiomers of

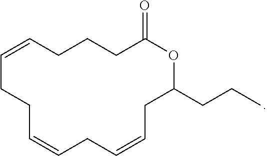 [SMILES: CCCC1C/C=C\C/C=C\CC/C=C\CCCC(=O)O1]

In certain embodiments, the mixture of two or more novel lactones is a mixture of the opposite enantiomers of

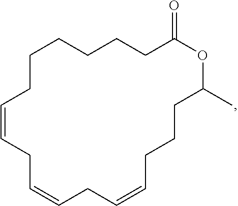 [SMILES: CC1CCC/C=C\C/C=C\C/C=C\CCCCCCC(=O)O1]

the opposite enantiomers of

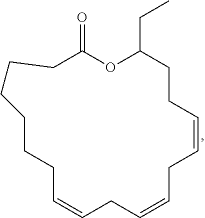 [SMILES: CCC1CC/C=C\C/C=C\C/C=C\CCCCCCC(=O)O1]

and the opposite enantiomers of

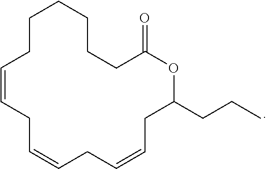 [SMILES: CCCC1C/C=C\C/C=C\C/C=C\CCCCCCC(=O)O1]

In certain embodiments, the mixture of two or more novel lactones is a mixture of the opposite enantiomers of

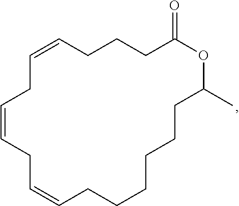 [SMILES: CC1CCCCCC/C=C\C/C=C\C/C=C\CCCC(=O)O1]

the opposite enantiomers of

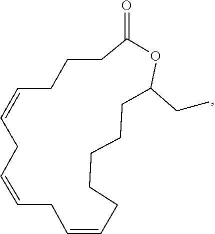 [SMILES: CCC1CCCCC/C=C\C/C=C\C/C=C\CCCC(=O)O1]

and the opposite enantiomers of

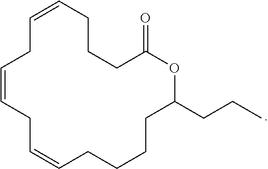 [SMILES: CCCC1CCCC/C=C\C/C=C\C/C=C\CCCC(=O)O1]

In certain embodiments, the mixture of two or more novel lactones is a mixture of the opposite enantiomers of

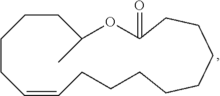 [SMILES: CC1CCCC/C=C\CCCCCCCCC(=O)O1]

the opposite enantiomers of

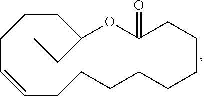 [SMILES: CCC1CCC/C=C\CCCCCCCCC(=O)O1]

and the opposite enantiomers of

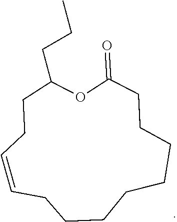 [SMILES: CCCC1CC/C=C\CCCCCCCCC(=O)O1]

In certain embodiments, the mixture of two or more novel lactones is a mixture of the opposite enantiomers of

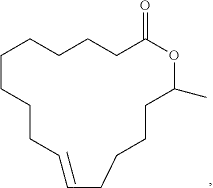

the opposite enantiomers of

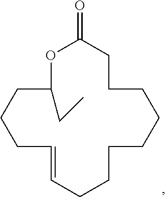 [SMILES: CCC1CCC/C=C/CCCCCCCCC(=O)O1]

and the opposite enantiomers of

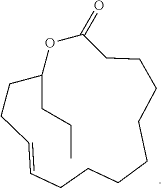 [SMILES: CCCC1CC/C=C/CCCCCCCCC(=O)O1]

In certain embodiments, the mixture of two or more novel lactones is a mixture of the opposite enantiomers of

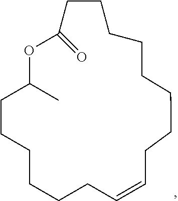 [SMILES: CC1CCCCCC/C=C\CCCCCCCCC(=O)O1]

the opposite enantiomers of

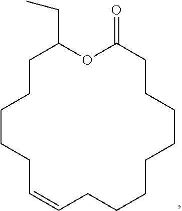 [SMILES: CCC1CCCCC/C=C\CCCCCCCCC(=O)O1]

and the opposite enantiomers of

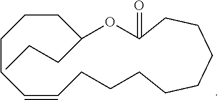 [SMILES: CCCC1CCCC/C=C\CCCCCCCCC(=O)O1]

In certain embodiments, the mixture of two or more novel lactones is a mixture of the opposite enantiomers of

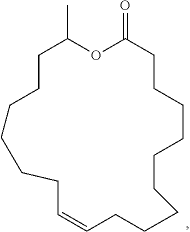 [SMILES: CC1CCCCCC/C=C\CCCCCCCCCC(=O)O1]

the opposite enantiomers of

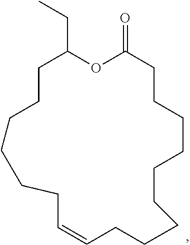 [SMILES: CCC1CCCCCC/C=C\CCCCCCCCCC(=O)O1]

and the opposite enantiomers of

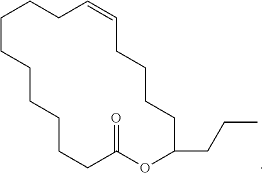 [SMILES: CCCC1CCCC/C=C\CCCCCCCCCC(=O)O1]

### Synthetic Biology

Standard recombinant DNA and molecular cloning techniques used here are well known in the art and are described, for example, by Sambrook, J., Fritsch, E. F. and Maniatis, T. MOLECULAR CLONING: A LABORATORY MANUAL, 2nd ed.; Cold Spring Harbor Laboratory: Cold Spring Harbor, N.Y., 1989 (hereinafter “Maniatis”); and by Silhavy, T. J., Bennan, M. L. and Enquist, L. W. EXPERIMENTS WITH GENE FUSIONS; Cold Spring Harbor Laboratory: Cold Spring Harbor, N.Y., 1984; and by Ausubel, F. M. et al., IN CURRENT PROTOCOLS IN MOLECULAR BIOLOGY, published by GREENE PUBLISHING AND WILEY-INTERSCIENCE, 1987; (the entirety of each of which is hereby incorporated herein by reference).

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred materials and methods are described below.

The disclosure will be more fully understood upon consideration of the following non-limiting Examples. It should be understood that these Examples, while indicating preferred embodiments of the subject technology, are given by way of illustration only. From the above discussion and these Examples, one skilled in the art can ascertain the essential characteristics of the subject technology, and without departing from the spirit and scope thereof, can make various changes and modifications of the subject technology to adapt it to various uses and conditions.

### Bacterial Production Systems

Expression of proteins in prokaryotes is most often carried out in a bacterial host cell with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (1) to increase expression of recombinant protein; (2) to increase the solubility of the recombinant protein; and (3) to aid in the purification of the recombinant protein by acting as a ligand in affinity purification. Often, a proteolytic cleavage site is introduced at the junction of the fusion moiety and the recombinant protein to enable separation of the recombinant protein from the fusion moiety subsequent to purification of the fusion protein. Such vectors are within the scope of the present disclosure.

In an embodiment, the expression vector includes those genetic elements for expression of the recombinant polypeptide in bacterial cells. The elements for transcription and translation in the bacterial cell can include a promoter, a coding region for the protein complex, and a transcriptional terminator. We only used one standard E. coli expression system for this proof-of-concept work. The further modification and optimization is in progress.

A person of ordinary skill in the art will be aware of the molecular biology techniques available for the preparation of expression vectors. The polynucleotide used for incorporation into the expression vector of the subject technology, as described above, can be prepared by routine techniques such as polymerase chain reaction (PCR).

A number of molecular biology techniques have been developed to operably link DNA to vectors via complementary cohesive termini. In one embodiment, complementary homopolymer tracts can be added to the nucleic acid molecule to be inserted into the vector DNA. The vector and nucleic acid molecule are then joined by hydrogen bonding between the complementary homopolymeric tails to form recombinant DNA molecules.

In an alternative embodiment, synthetic linkers containing one or more restriction sites provide are used to operably link the polynucleotide of the subject technology to the expression vector. In an embodiment, the polynucleotide is generated by restriction endonuclease digestion. In an embodiment, the nucleic acid molecule is treated with bacteriophage T4 DNA polymerase or E. coli DNA polymerase I, enzymes that remove protruding, 3′-single-stranded termini with their 3′-5′-exonucleolytic activities and fill-in recessed 3′-ends with their polymerizing activities, thereby generating blunt-ended DNA segments. The blunt-ended segments are then incubated with a large molar excess of linker molecules in the presence of an enzyme that is able to catalyze the ligation of blunt-ended DNA molecules, such as bacteriophage T4 DNA ligase. Thus, the product of the reaction is a polynucleotide carrying polymeric linker sequences at its ends. These polynucleotides are then cleaved with the appropriate restriction enzyme and ligated to an expression vector that has been cleaved with an enzyme that produces termini compatible with those of the polynucleotide.

Alternatively, a vector having ligation-independent cloning (LIC) sites can be employed. The required PCR amplified polynucleotide can then be cloned into the LIC vector without restriction digest or ligation (Aslanidis and de Jong, NUCL. ACID. RES. 18 6069-74, (1990), Haun, et al, BIOTECHNIQUES 13, 515-18 (1992), which is incorporated herein by reference to the extent it is consistent herewith).

In an embodiment, in order to isolate and/or modify the polynucleotide of interest for insertion into the chosen plasmid, it is suitable to use PCR. Appropriate primers for use in PCR preparation of the sequence can be designed to isolate the required coding region of the nucleic acid molecule, add restriction endonuclease or LIC sites, place the coding region in the desired reading frame.

In an embodiment, a polynucleotide for incorporation into an expression vector of the subject technology is prepared by the use of PCR using appropriate oligonucleotide primers. The coding region is amplified, whilst the primers themselves become incorporated into the amplified sequence product. In an embodiment, the amplification primers contain restriction endonuclease recognition sites, which allow the amplified sequence product to be cloned into an appropriate vector.

The expression vectors can be introduced into plant or microbial host cells by conventional transformation or transfection techniques. Transformation of appropriate cells with an expression vector of the subject technology is accomplished by methods known in the art and typically depends on both the type of vector and cell. Suitable techniques include calcium phosphate or calcium chloride co-precipitation, DEAE-dextran mediated transfection, lipofection, chemoporation or electroporation.

Successfully transformed cells, that is, those cells containing the expression vector, can be identified by techniques well known in the art. For example, cells transfected with an expression vector of the subject technology can be cultured to produce polypeptides described herein. Cells can be examined for the presence of the expression vector DNA by techniques well known in the art.

The host cells can contain a single copy of the expression vector described previously, or alternatively, multiple copies of the expression vector.

In some embodiments, the transformed cell is an animal cell, an insect cell, a plant cell, an algal cell, a fungal cell, or a yeast cell. In some embodiments, the cell is a plant cell selected from the group consisting of: canola plant cell, a rapeseed plant cell, a palm plant cell, a sunflower plant cell, a cotton plant cell, a corn plant cell, a peanut plant cell, a flax plant cell, a sesame plant cell, a soybean plant cell, and a petunia plant cell.

Microbial host cell expression systems and expression vectors containing regulatory sequences that direct high-level expression of foreign proteins are well known to those skilled in the art. Any of these could be used to construct vectors for expression of the recombinant polypeptide of the subjection technology in a microbial host cell. These vectors could then be introduced into appropriate microorganisms via transformation to allow for high level expression of the recombinant polypeptide of the subject technology.

Vectors or cassettes useful for the transformation of suitable microbial host cells are well known in the art. Typically the vector or cassette contains sequences directing transcription and translation of the relevant polynucleotide, a selectable marker, and sequences allowing autonomous replication or chromosomal integration. Suitable vectors comprise a region 5′ of the polynucleotide which harbors transcriptional initiation controls and a region 3′ of the DNA fragment which controls transcriptional termination. It is preferred for both control regions to be derived from genes homologous to the transformed host cell, although it is to be understood that such control regions need not be derived from the genes native to the specific species chosen as a host.

Initiation control regions or promoters, which are useful to drive expression of the recombinant polypeptide in the desired microbial host cell are numerous and familiar to those skilled in the art. Virtually any promoter capable of driving these genes is suitable for the subject technology including but not limited to CYCI, HIS3, GALI, GALIO, ADHI, PGK, PH05, GAPDH, ADCI, TRPI, URA3, LEU2, ENO, TPI (useful for expression in Saccharomyces); AOXI (useful for expression in Pichia); and lac, trp, JPL, IPR, T7, tac, and trc (useful for expression in Escherichia coli).

Termination control regions may also be derived from various genes native to the microbial hosts. A termination site optionally may be included for the microbial hosts described herein.

In plant cells, the expression vectors of the subject technology can include a coding region operably linked to promoters capable of directing expression of the recombinant polypeptide of the subject technology in the desired tissues at the desired stage of development. For reasons of convenience, the polynucleotides to be expressed may comprise promoter sequences and translation leader sequences derived from the same polynucleotide. 3′ non-coding sequences encoding transcription termination signals should also be present. The expression vectors may also comprise one or more introns in order to facilitate polynucleotide expression.

For plant host cells, any combination of any promoter and any terminator capable of inducing expression of a coding region may be used in the vector sequences of the subject technology. Some suitable examples of promoters and terminators include those from nopaline synthase (nos), octopine synthase (ocs) and cauliflower mosaic virus (CaMV) genes. One type of efficient plant promoter that may be used is a high-level plant promoter. Such promoters, in operable linkage with an expression vector of the subject technology should be capable of promoting the expression of the vector. High level plant promoters that may be used in the subject technology include the promoter of the small subunit (s) of the ribulose-1,5-bisphosphate carboxylase for example from soybean (Berry-Lowe et al., J. MOLECULAR AND APP. GEN., 1:483 498 (1982), the entirety of which is hereby incorporated herein to the extent it is consistent herewith), and the promoter of the chlorophyll binding protein. These two promoters are known to be light-induced in plant cells (see, for example, GENETIC ENGINEERING OF PLANTS, AN AGRICULTURAL PERSPECTIVE, A. Cashmore, Plenum, N.Y. (1983), pages 29 38; Coruzzi, G. et al., The Journal of Biological CHEMISTRY, 258: 1399 (1983), and Dunsmuir, P. et al., JOURNAL OF MOLECULAR AND APPLIED GENETICS, 2:285 (1983), each of which is hereby incorporated herein by reference to the extent they are consistent herewith).

One with skill in the art will recognize that the lactone composition(s) produced by the methods described herein can be further purified and mixed with other lactones, flavors, or scents to obtain a desired composition for use in a variety of consumer products or foods.

### Analysis of Sequence Similarity Using Identity Scoring

As used herein “sequence identity” refers to the extent to which two optimally aligned polynucleotide or peptide sequences are invariant throughout a window of alignment of components, e.g., nucleotides or amino acids. An “identity fraction” for aligned segments of a test sequence and a reference sequence is the number of identical components which are shared by the two aligned sequences divided by the total number of components in reference sequence segment, i.e., the entire reference sequence or a smaller defined part of the reference sequence.

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned (with appropriate nucleotide insertions, deletions, or gaps totaling less than 20 percent of the reference sequence over the window of comparison). Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and preferably by computerized implementations of these algorithms such as GAP, BESTFIT, FASTA, and TFASTA available as part of the GCG® Wisconsin Package® (Accelrys Inc., Burlington, MA). An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this disclosure “percent identity” may also be determined using BLASTX version 2.0 for translated nucleotide sequences and BLASTN version 2.0 for polynucleotide sequences.

The percent of sequence identity is preferably determined using the “Best Fit” or “Gap” program of the Sequence Analysis Software Package™ (Version 10; Genetics Computer Group, Inc., Madison, WI). “Gap” utilizes the algorithm of Needleman and Wunsch (Needleman and Wunsch, JOURNAL OF MOLECULAR BIOLOGY 48:443-453, 1970) to find the alignment of two sequences that maximizes the number of matches and minimizes the number of gaps. “BestFit” performs an optimal alignment of the best segment of similarity between two sequences and inserts gaps to maximize the number of matches using the local homology algorithm of Smith and Waterman (Smith and Waterman, ADVANCES IN APPLIED MATHEMATICS, 2:482-489, 1981, Smith et al., NUCLEIC ACIDS RESEARCH 11:2205-2220, 1983). The percent identity is most preferably determined using the “Best Fit” program.

Useful methods for determining sequence identity are also disclosed in the Basic Local Alignment Search Tool (BLAST) programs which are publicly available from National Center Biotechnology Information (NCBI) at the National Library of Medicine, National Institute of Health, Bethesda, Md. 20894; see BLAST Manual, Altschul et al., NCBI, NLM, NIH; Altschul et al., J. MOL. BIOL. 215:403-410 (1990); version 2.0 or higher of BLAST programs allows the introduction of gaps (deletions and insertions) into alignments; for peptide sequence BLASTX can be used to determine sequence identity; and, for polynucleotide sequence BLASTN can be used to determine sequence identity.

As used herein, the term “substantial percent sequence identity” refers to a percent sequence identity of at least about 70% sequence identity, at least about 80% sequence identity, at least about 85% identity, at least about 90% sequence identity, or even greater sequence identity, such as about 98% or about 99% sequence identity. Thus, one embodiment of the disclosure is a polynucleotide molecule that has at least about 70% sequence identity, at least about 80% sequence identity, at least about 85% identity, at least about 90% sequence identity, or even greater sequence identity, such as about 98% or about 99% sequence identity with a polynucleotide sequence described herein. Polynucleotide molecules that have the activity of the Blu1 and Cytochrome P450 genes of the current disclosure are capable of directing the production of a variety of lactones, such as macrocyclic lactones, and have a substantial percent sequence identity to the polynucleotide sequences provided herein and are encompassed within the scope of this disclosure.

Identity is the fraction of amino acids that are the same between a pair of sequences after an alignment of the sequences (which can be done using only sequence information or structural information or some other information, but usually it is based on sequence information alone), and similarity is the score assigned based on an alignment using some similarity matrix. The similarity index can be any one of the following BLOSUM62, PAM250, or GONNET, or any matrix used by one skilled in the art for the sequence alignment of proteins.

Identity is the degree of correspondence between two sub-sequences (no gaps between the sequences). An identity of 25% or higher implies similarity of function, while 18-25% implies similarity of structure or function. Keep in mind that two completely unrelated or random sequences (that are greater than 100 residues) can have higher than 20% identity. Similarity is the degree of resemblance between two sequences when they are compared. This is dependent on their identity.

As is evident from the foregoing description, certain aspects of the present disclosure are not limited by the particular details of the examples illustrated herein, and it is therefore contemplated that other modifications and applications, or equivalents thereof, will occur to those skilled in the art. It is accordingly intended that the claims shall cover all such modifications and applications that do not depart from the spirit and scope of the present disclosure.

Moreover, unless defined otherwise, all technical and scientific terms used herein have the same meaning as commonly understood by one of ordinary skill in the art to which the disclosure belongs. Although any methods and materials similar to or equivalent to or those described herein can be used in the practice or testing of the present disclosure, the preferred methods and materials are described above.

### EXAMPLES

### Example 1

In this process, a cytochrome P450 enzyme, e.g. P450 BM3 (Miura and Fulco, 1975) that has fatty acid subterminal hydroxylase activity, was used to make ω-1, ω-2 or ω-3 hydroxyl fatty acids or their mixtures (Process 1, Miura Y, Fulco A J. (1975) Omega-1, Omega-2 and Omega-3 hydroxylation of long-chain fatty acids, amides and alcohols by a soluble enzyme system from Bacillus megaterium. Biochim. Biophys. Acta. 388: 305-317).

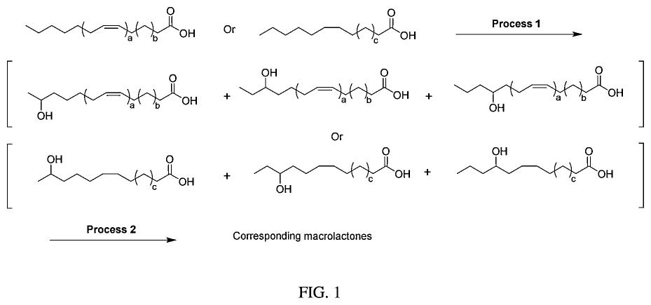

The hydroxyl fatty acid products may be further processed by lipase-catalyzed ring closure reaction in organic solvents for the formation of branched-chain musky macrolactones (Process 2). The overall synthetic scheme is illustrated in FIG. 1.

Process 1 involves a fatty acid hydroxylation by subjecting the saturated or unsaturated fatty acid to the action of a biological catalyst (hydroxylase) present in a fermentation media. The hydroxyl fatty acid obtained from Process 1 may be isolated from the fermentation broth (for example by liquid-liquid extraction using a mixture of hydrocarbon-based organic solvents such as hexane and a water-insoluble polar solvent such as ethyl acetate).

Process 2 involves a reaction of obtaining macrolactones by subjecting the hydroxyl fatty acids to an intramolecular macrolactonization reaction catalyzed by a lipase enzyme. The lipase enzyme may be immobilized on a solid support and used for the macrolacronization process. The immobilized lipase can be easily recovered and recycled repeatedly for a long term.

Process 2 can be carried out in the presence of immobilized lipase catalyst such as Novozyme 435 in a solvent such as toluene of dichloroethane. The concentration of hydroxyl fatty acid is adjusted at 0.05 to 0.025 molar. The concentration of immobilized lipase enzyme may be appropriately selected to amounts that do not decrease the reaction rate. In the case of using Novozyme 435, it is preferable to adjust the concentration at 50 to 100 grams per liter. The reaction is usually shaken and stirred for about 15 to 24 hours at 40 to 60° C.

The target macrolactones can be isolated from the reaction mixture using a proper combination of conventional purification techniques such as filtration, chromatography, and drying.

### Example 2

A total number of eight fatty acids were used for musk lactone production.

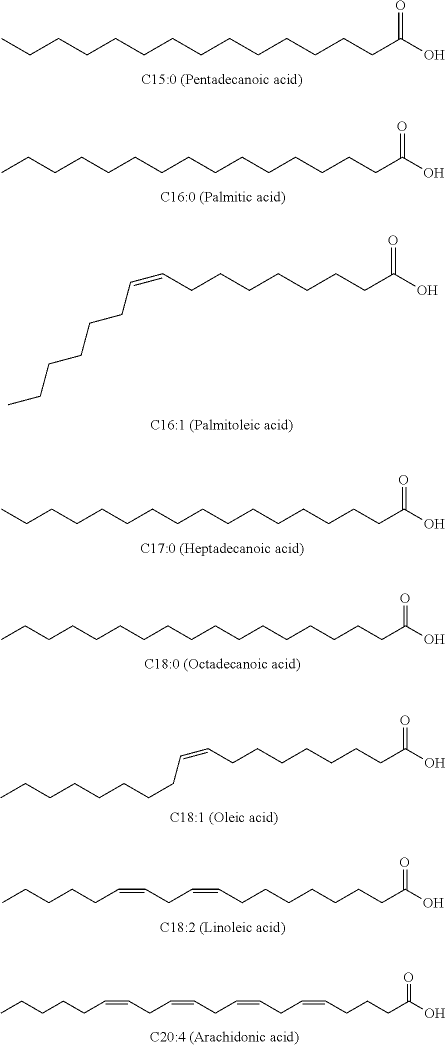

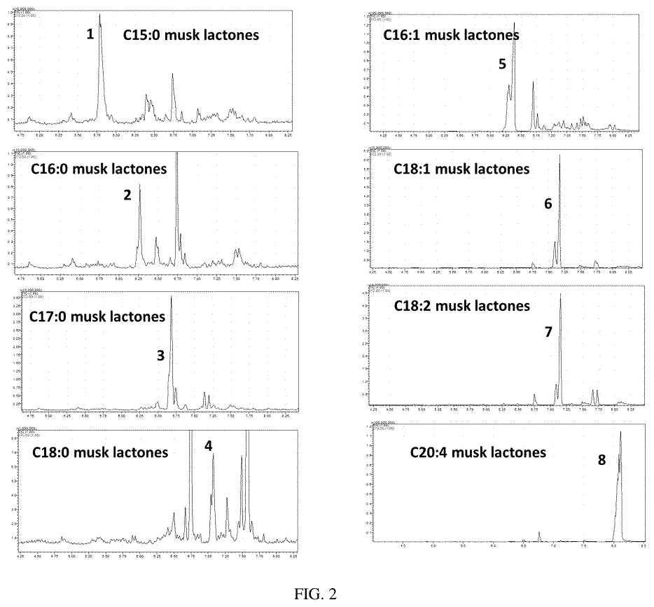

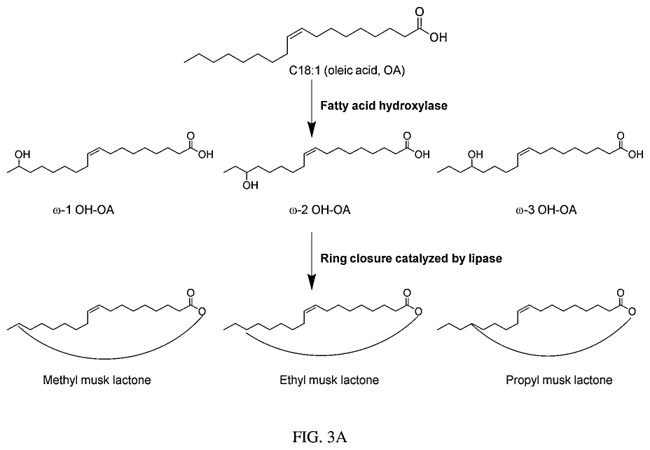

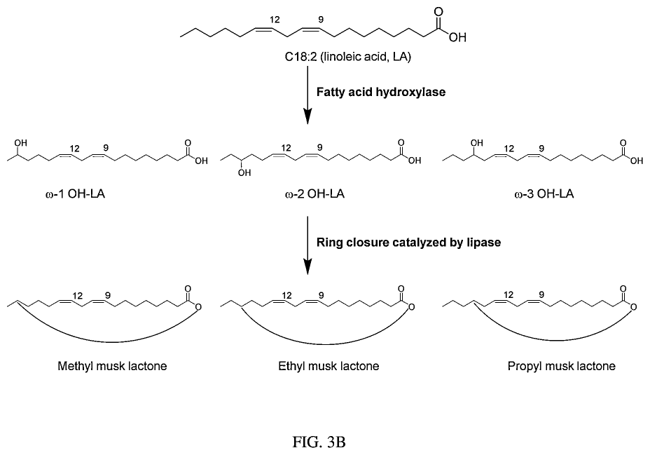

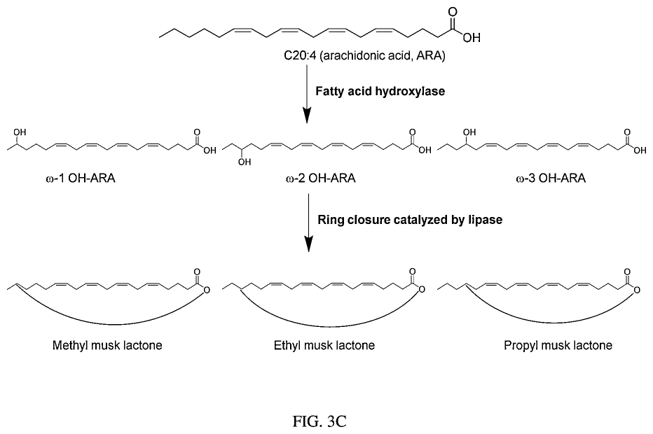

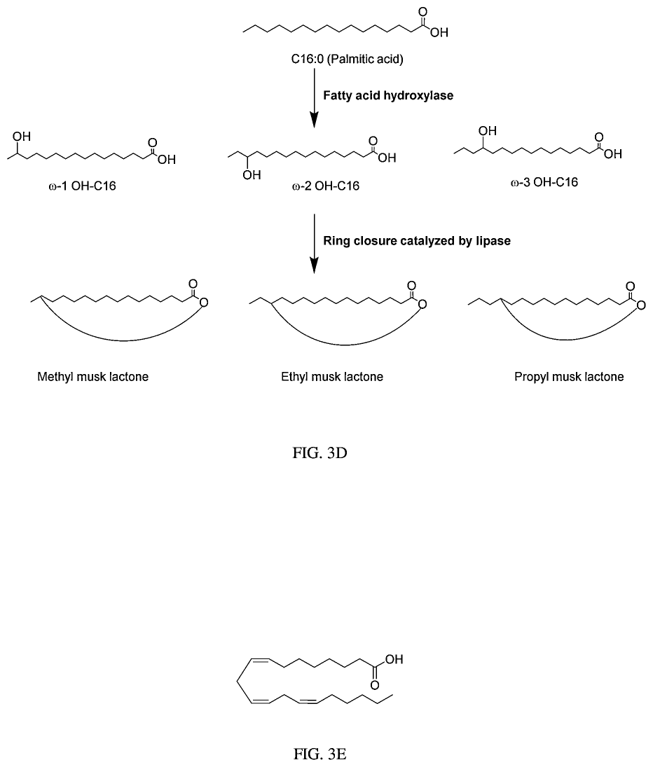

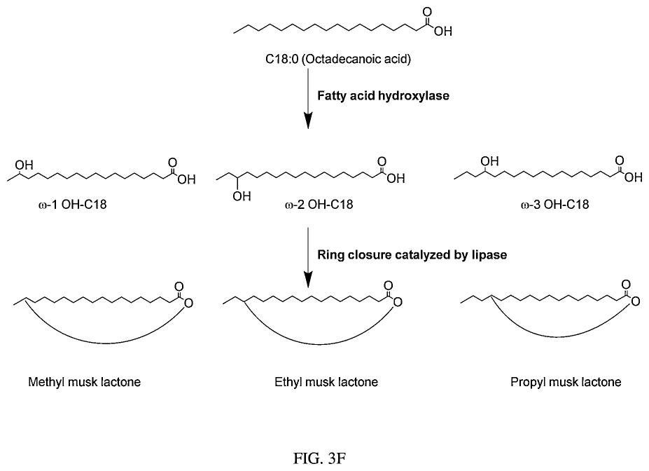

After hydroxylation and ring closure, eight different musk lactone mixtures were produced from these eight fatty acids, respectively as shown in FIG. 2. The eight fatty acids and corresponding musk lactone products is shown in Table 1. Reaction schemes showing fatty acid hydroxylation and ring closure are illustrated for oleic acid, linoleic acid, arachidonic acid, palmitic acid, heptadecanoic acid, and octadecanoic acid in FIGS. 3A-3F, respectively.

TABLE 1

| | | | MuL lactone |
|---|---|---|---|
| # | Parent Fatty Acid | MuL lactone product | product 2 |
| 1 | 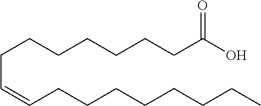 [SMILES: CCCCCCCC/C=C\CCCCCCCC(=O)O] C18:1, Oleic Acid (OA) | 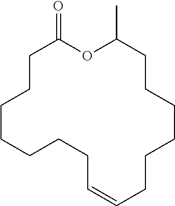 [SMILES: CC1CCCCCC/C=C\CCCCCCCC(=O)O1] | 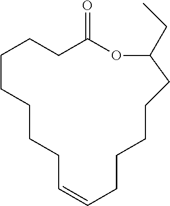 [SMILES: CCC1CCCCC/C=C\CCCCCCCC(=O)O1] |
| | | (Z)-18- | (Z)-17- |
| | | methyloxacyclooctadec- | ethyloxacycloheptadec- |
| | | 10-en-2-one | 10-en-2-one |
| | | racemic, CAS 2055033- | |
| | | 69-5 | |
| | | (18R), CAS 2055033-71-9 | |
| | | (18S), CAS 2055033-70-8 | |
| 2 | 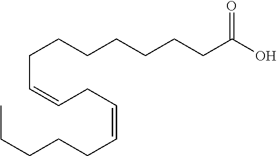 [SMILES: CCCCC/C=C\C/C=C\CCCCCCCC(=O)O] C18:2, Linoleic acid (LA) | 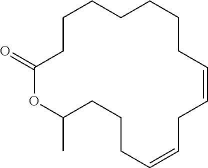 [SMILES: CC1CCC/C=C\C/C=C\CCCCCCCC(=O)O1] (10Z, 13Z)-18- methyloxacyclooctadeca- 10,13-dien-2-one | 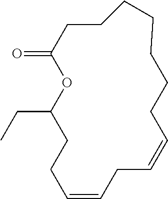 [SMILES: CCC1CC/C=C\C/C=C\CCCCCCCC(=O)O1] (10Z, 13Z)-17- ethyloxacycloheptadeca- |
| | | | 10,13-dien-2-one |
| 3 | 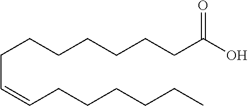 [SMILES: CCCCCC/C=C\CCCCCCCC(=O)O] C16:1, Palmitoleic acid (A) | 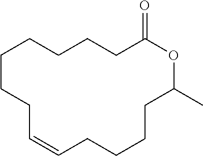 [SMILES: CC1CCCC/C=C\CCCCCCCC(=O)O1] (Z)-16- methyloxacyclohexadec- | 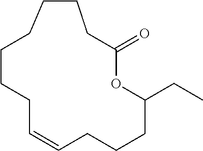 [SMILES: CCC1CCC/C=C\CCCCCCCC(=O)O1] (Z)-15- ethyloxacyclopentadec- 10-en-2-one |
| | | 10-en-2-one | |
| 4 | 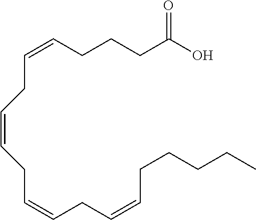 [SMILES: CCCCC/C=C\C/C=C\C/C=C\C/C=C\CCCC(=O)O] | 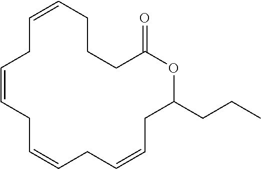 [SMILES: CCCC1C/C=C\C/C=C\C/C=C\C/C=C\CCCC(=O)O1] (6Z, 9Z, 12Z, 15Z)-18- propyloxacyclooctadeca- 6, 9, 12, 15-tetraen-2-one | 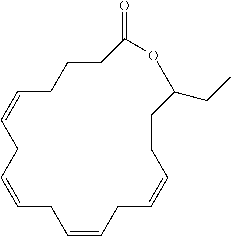 [SMILES: CCC1CC/C=C\C/C=C\C/C=C\C/C=C\CCCC(=O)O1] |
| | C20:4, Arachidonic acid | | |
| | (AA) | | (6Z, 9Z, 12Z, 15Z)-19- |
| | | | ethyloxacyclononade |
| | | | ca-6, 9, 12, 15- |
| | | | tetraen-2-one |
| 5 | 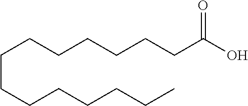 [SMILES: CCCCCCCCCCCCCCC(=O)O] C15:0, pentadecanoic acid | 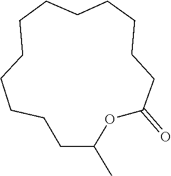 [SMILES: CC1CCCCCCCCCCCCC(=O)O1] 15-methyloxacyclopentadecan-2-one racemic, CAS 32539-85-8 (15R) - CAS 69297-55-8 (15S) - CAS 206124-09-6 | 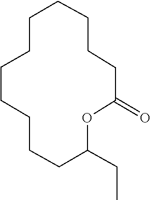 [SMILES: CCC1CCCCCCCCCCCC(=O)O1] 14-ethyloxacyclotetradecan-2-one racemic, CAS 4723-83-5 (14R)-CAS 239801-19-5 |
| | | | (14S)-CAS 239801-33-3 |
| 6 | 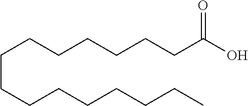 [SMILES: CCCCCCCCCCCCCCCC(=O)O] C16:0, hexadecanoic acid (palmitic acid) | 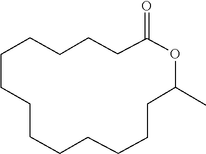 [SMILES: CC1CCCCCCCCCCCCCC(=O)O1] 16-methyloxacyclohexadecan-2-one, racemic, CAS 4459-57-8 (16R), CAS 69297-56-9 (16S), CAS 129214-002 | 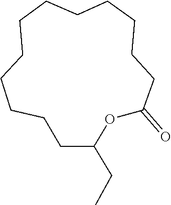 [SMILES: CCC1CCCCCCCCCCCCC(=O)O1] 15-ethyloxacyclopentadecan-2-one racemic, CAS 140389-84-0 |
| | | | 71-4 |
| 7 | 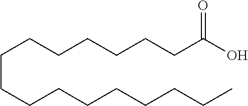 [SMILES: CCCCCCCCCCCCCCCCC(=O)O] C17:0, heptadecanoic acid | 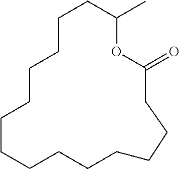 [SMILES: CC1CCCCCCCCCCCCCCC(=O)O1] 17-methyloxacycloheptadecan-2-one CAS 111908-52-2 | 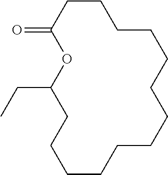 [SMILES: CCC1CCCCCCCCCCCCCC(=O)O1] 16- ethyloxacyclohexadecan-2-one |
| | | | racemic, CAS 89426-66-4 |
| | | | (16R) CAS 239801-26-4 |
| | | | (16S) CAS 239801-36-6 |
| 8 | 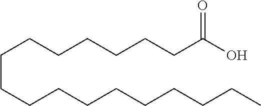 [SMILES: CCCCCCCCCCCCCCCCCC(=O)O] C18:0, octadecanoic acid | 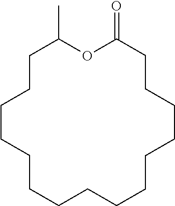 [SMILES: CC1CCCCCCCCCCCCCCCC(=O)O1] 18-methyloxacyclooctadecan-2-one | 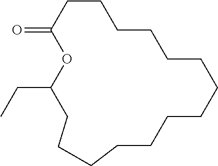 [SMILES: CCC1CCCCCCCCCCCCCCC(=O)O1] 17-ethyloxacycloheptadecan-2-one CAS 140389-85-1 |
| | | CAS 111879-77-7 | |

| | MuL lactone |
|---|---|
| # | product 3 |
| 1 | 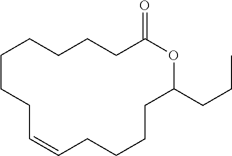 [SMILES: CCCC1CCCC/C=C\CCCCCCCC(=O)O1] |
| | (Z)-16- |
| | propyloxacyclohexadec- |
| | 10-en-2-one |
| 2 | 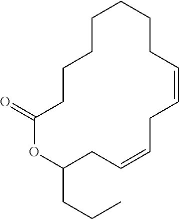 [SMILES: CCCC1C/C=C\C/C=C\CCCCCCCC(=O)O1] (10Z, 13Z)-16- |
| | propyloxacyclohexadeca- |
| | 10,13-dien-2-one |
| | (16R), |
| | CAS 1809795-94-5 |
| | (16S), |
| | CAS 1809894-22-1 |
| 3 | 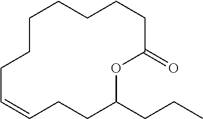 [SMILES: CCCC1CC/C=C\CCCCCCCC(=O)O1] (Z)-14- |
| | propyloxacyclotetradec- |
| | 10-en-2-one |
| 4 | 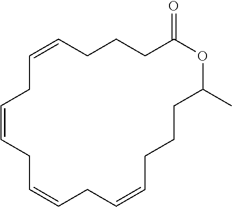 [SMILES: CC1CCC/C=C\C/C=C\C/C=C\C/C=C\CCCC(=O)O1] |
| | (6Z, 9Z, 12Z, 15Z)-20- |
| | methyloxacycloicosa-6, |
| | 9, 12, 15-tetraen-2-one |
| 5 | 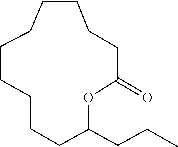 [SMILES: CCCC1CCCCCCCCCCC(=O)O1] |
| | 13- |
| | propyloxacyclotridecan-2-one |
| | CAS 89328-31-4 |
| 6 | 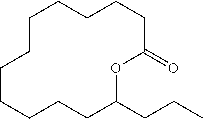 [SMILES: CCCC1CCCCCCCCCCCC(=O)O1] |
| | 14-propyloxacyclotetradecan-2-one |
| | CAS 140390-02-9 |
| 7 | 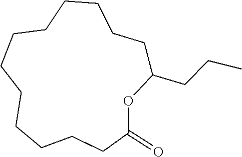 [SMILES: CCCC1CCCCCCCCCCCCC(=O)O1] |
| | 15- |
| | propyloxacyclopentadecan- |
| | 2-one |
| 8 | 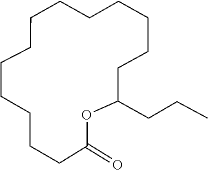 [SMILES: CCCC1CCCCCCCCCCCCCC(=O)O1] |
| | 16- |
| | propyloxacyclopentadecan- |
| | 2-one |
| | CAS 140390-03-0 |

### Fatty Acid Hydroxylation

The amino acid sequence of a BM3 homolog of Myceliophthora thermophile (CYP505A30) was obtained from UniProt (uniprot.org/uniprot/G2QDZ3.fasta) and the corresponding gene was codon optimized for expression in Escherichia coli and synthesized by GenScript (Piscataway, NJ).

The resulting gene product was cloned into pETDuet-1 vector (AMP+, Novagen) through NdeI and XhoI sites. The construct was transformed into BL21(DE3) cells for expression.

In a typical experiment, an overnight culture was used to inoculate liquid LB medium (2%) containing 100 mg/L of carbenicillin and 0.4 mM 5-aminolevulinic acid. The culture was first grown at 37° C. to an OD600 of 0.6 and cooled down to 16° C. Then 1 mM IPTG was added to induce protein expression. After 16 hours of incubation at 16° C., cells were harvested by centrifugation.

Harvested cell pellets were re-suspended at a concentration of 100 g/L fresh weight in 100 mM potassium phosphate buffer (pH7.0) containing 0.1% Tween 40 and 10 mM NADPH. Then 1 g/L of fatty acid substrate was added. The mixture was shaken at 37° C. in a shaker.

Production of Branched-Chain Musky Macrolactones from Palmitic Acid

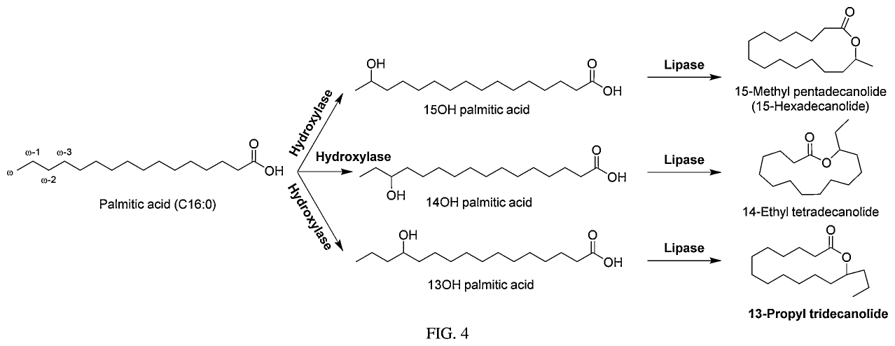

The overall synthetic scheme showing palmitic acid hydroxylation and a reaction of obtaining corresponding musk lactone products is illustrated in FIG. 4. Harvested cell pellets were re-suspended at a concentration of 100 g/L fresh weight in 100 mM potassium phosphate buffer (pH7.0) containing 0.1% Tween 40 and 10 mM NADPH. Then 1 g/L of palmitic acid (C16:0) was added. The mixture was shaken at 37° C. in a shaker.

Hydroxylated palmitic acids were extracted by ethyl acetate and ethyl acetate phase was dried in SpeedVac™ vacuum concentrator. Then Novozym 435 in the form of acrylic resin (Sigma) was added with toluene as solvent for ring closure reaction at 60° C. with shaking. The products from hydroxyl palmitic acids were analyzed by GC/MS.

GC/MS analysis was conducted on Shimadzu GC-2010 system coupled with GCMS-QP2010S detector. The analytical column is SHRXI-5MS (thickness 0.25 μm; length 30 m; diameter 0.25 mm) and the injection temperature is 265° C. under split mode. The temperature gradient is 0-3 min 150° C.; 3-6.7 min 150° C. to 260° C., a gradient of 30; 6.7-15.7 min, 260° C.

Production of Branched-Chain Musky Macrolactones from Oleic Acid

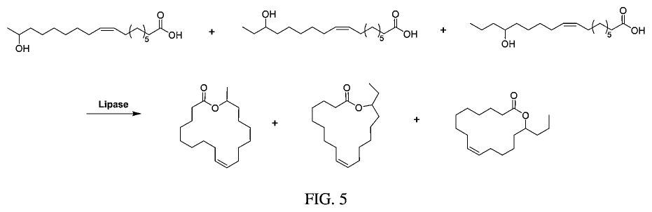

First, oleic acid was hydroxylated to form hydroxyl fatty acid, as outlined in Process 1 in Example 1. Thereafter, the hydroxyl fatty acid were cyclized as illustrated in FIG. 5 (Process 2).

3 liters of fermentation broth containing hydroxyl oleic acid analogs were extracted with 11 liters of 50% ethyl acetate-hexane in 1-liter batches. The solvents were distilled off under reduced pressure to obtain 11.50 g of crude extract as a reddish oil. The crude extract was purified by column chromatography on silica gel eluting with 20 to 50% ethyl acetate-hexane to obtain 1.40 g of hydroxyl oleic acid isomers.

1.40 g (4.66 mmol) of hydroxyl oleic acid analogs was dissolved in 200 ml toluene. 20 g of Novozyme 435 was added to this solution. The reaction mixture was heated to 55° C. and stirred for 15 hours then cooled to room temperature. The immobilized lipase enzyme was separated by filtration and rinsed with dichloromethane. The volatiles were distilled off under reduced pressure to obtain 1.50 g of crude product as a yellow oil. The crude product was purified by column chromatography on silica gel eluting with 5% ethyl acetate-hexane to obtain 1.10 g (3.92 mmol, 62.7% isolated yield, 98% total purity of three isomers) of corresponding macrolactonization products.

It was confirmed by a sensory evaluation that the mixture of the three components has a luxurious and novel musk fragrance.

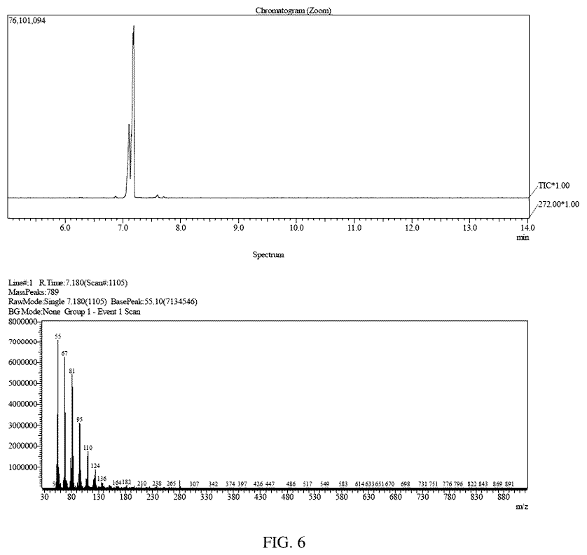

A 1 mg/ml solution of the purified product in hexane was analyzed on a capillary gas chromatograph-mass spectrometer GCMS-QP2020 NX (manufactured by Shimadzu) using a 30 m×0.25 mm 0.25 μm Rtx-5MS (manufactured by Restek). The analysis was carried out using high purity helium for the mobile phase, at a flow rate of 1 mL/min. The temperature program used was 150° C. for 3 minutes, a temperature gradient of 50° C./minute to 260° C., and isothermic at 260° C. for 9 minutes. Results are shown in FIG. 6.

Production of Branched-Chain Musky Macrolactones from Linoleic Acid

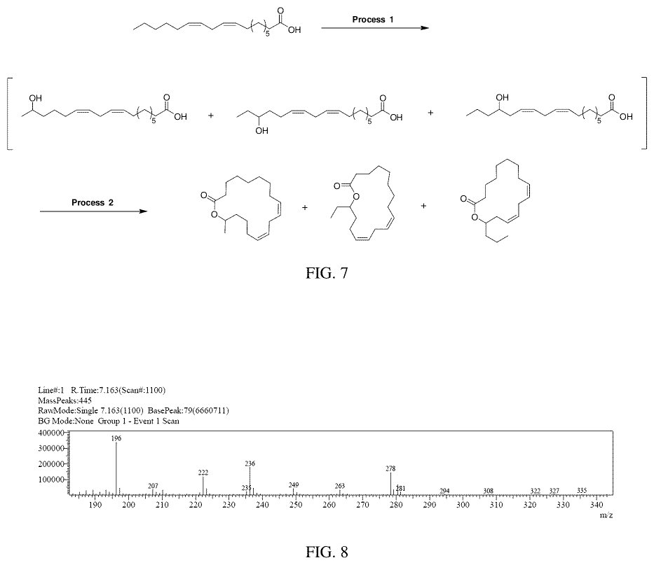

The overall synthetic scheme showing linoleic acid hydroxylation (Process 1) and a reaction of obtaining corresponding musk lactone products (Process 2) is illustrated in FIG. 7. 2 liters of fermentation broth containing hydroxyl linoleic acid analogs was extracted with 7 liters of 50% ethyl acetate-hexane in 0.7-liter batches. The solvents were distilled off under reduced pressure to obtain 18.5 g of crude extract as a reddish oil. The crude extract was purified by column chromatography on silica gel eluting with 20 to 50% ethyl acetate-hexane to obtain 1.2 g of hydroxyl linoleic acid isomers.

1.2 g (4.05 mmol) of hydroxyl linoleic acid analogs was dissolved in 160 ml toluene. 16 g of Novozyme 435 (100 g/L) was added to this solution. The reaction mixture was heated to 50° C. and stirred for 17 hours then cooled to room temperature. The immobilized lipase enzyme was separated by filtration and rinsed with dichloromethane. The volatiles were distilled off under reduced pressure to obtain 1.10 g of crude product as a yellow oil.

The crude product was purified by column chromatography on silica gel eluting with 5% ethyl acetate-hexane to obtain 0.70 g (2.52 mmol, 62.2% isolated yield, 95% total purity of three isomers) of corresponding macrolactonization products.

A 1 mg/ml solution of the purified product in hexane was analyzed on a capillary gas chromatograph-mass spectrometer GCMS-QP2020 NX (manufactured by Shimadzu) using a 30 m×0.25 mm 0.25 μm Rtx-5MS (manufactured by Restek). The analysis was carried out using high purity helium for the mobile phase, at a flow rate of 1 mL/min. The temperature program used was 150° C. for 3 minutes, a temperature gradient of 50° C./minute to 260° C., and isothermic at 260° C. for 9 minutes. Results are shown in FIG. 8.

### Example 3

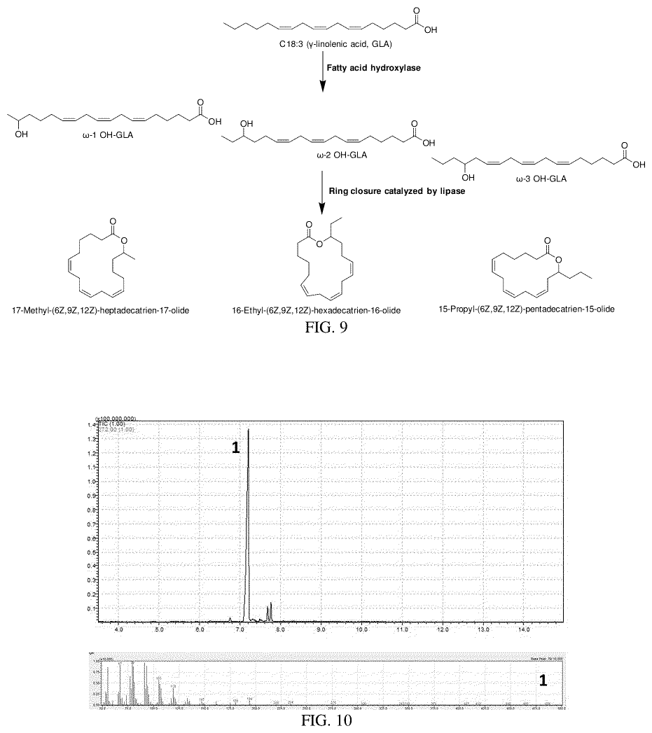

In this process, a cytochrome P450 enzyme e.g. P450 BM3 (Miura and Fulco, 1975; Wen and Fulco, 1987) or its homologs (Baker et. al, 2017) that have fatty acid subterminal hydroxylase activity to make ω-1, ω-2 or ω-3 hydroxy fatty acids or their mixtures was used. After extraction and purification, hyxroxy fatty acid were subject to lipase-catalyzed ring closure reaction in organic solvents for the formation of branched-chain musky macrolactones (FIG. 9).

The amino acid sequence of a BM3 homolog of Myceliophthora thermophile (CYP505A30) was obtained from UniProt (uniprot.org/uniprot/G2QDZ3.fasta) and the corresponding gene was codon optimized for expression in Escherichia coli and synthesized by GenScript (Piscataway, NJ).

The resulting gene product was cloned into pETDuet-1 vector (AMP+, Novagen) through NdeI and XhoI sites. The construct was transformed into BL21(DE3) cells for expression.

In a typical experiment, an overnight culture was used to inoculate liquid LB medium (2%) containing 100 mg/L of carbenicillin and 0.4 mM 5-aminolevulinic acid. The culture was first grown at 37° C. to an OD600 of 0.6 and cooled down to 16° C. Then 1 mM IPTG was added to induce protein expression. After 16 h of incubation at 16° C., cells were harvested by centrifugation.

Harvested cell pellets were re-suspended at a concentration of 100 g/L fresh weight in 100 mM potassium phosphate buffer (pH7.0) containing 0.1% Tween 40 and 10 mM NADPH. Then 1 g/L of γ-linolenic acid (GLA, C18:3) was added. The mixture was shaken at 37° C. in a shaker. Results of GC/MS analysis of musky lactones derived from hydroxy GLA (molecular weight: 276) are shown in FIG. 10.

Additional fatty acid substrates that may be used and the lactone products are provided below and shown in Table 2.

Production of Branched-Chain Musky Macrolactones from α-Linolenic Acid (ALA)

Harvested cell pellets were re-suspended at a concentration of 100 g/L fresh weight in 100 mM potassium phosphate buffer (pH7.0) containing 0.1% Tween 40 and 10 mM NADPH. Then 1 g/L of α-linolenic acid (ALA, C18:3) was added. The mixture was shaken at 37° C. in a shaker for 5 hours.

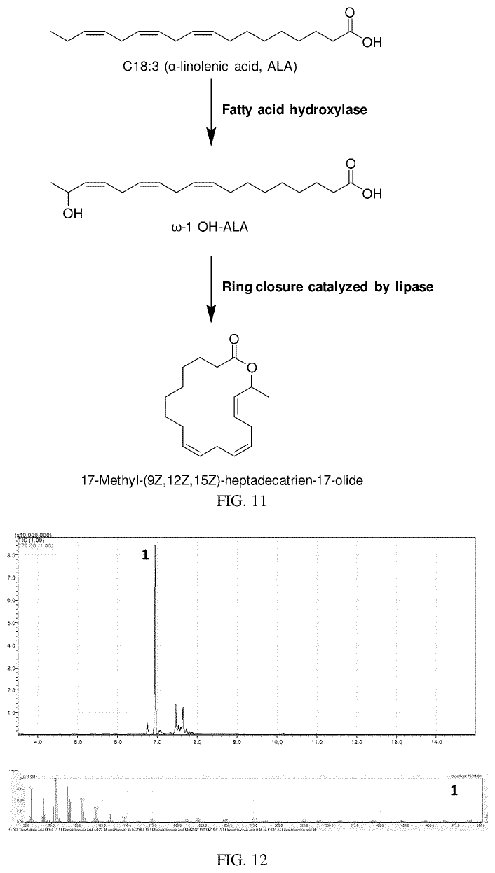

Hydroxylated α-linolenic acid was extracted by ethyl acetate and ethyl acetate phase was dried in SpeedVac™ vacuum concentrator. Then Novozym 435 in the form of acrylic resin (Sigma) was added with toluene as solvent for ring closure reaction at 60° C. with shaking. The products from hydroxy α-linolenic acid were analyzed by GC/MS. See FIGS. 11 and 12.

Production of Branched-Chain Musky Macrolactones from C20:3 (8Z,11Z,14Z-Eicosatrienoic Acid, Dihomo-γ-Linolenic Acid, DGLA)

Harvested cell pellets were re-suspended at a concentration of 100 g/L fresh weight in 100 mM potassium phosphate buffer (pH7.0) containing 0.1% Tween 40 and 10 mM NADPH. Then 1 g/L of dihomo-γ-linolenic acid (DGLA, C20:3) was added. The mixture was shaken at 37° C. in a shaker for 5 hours.

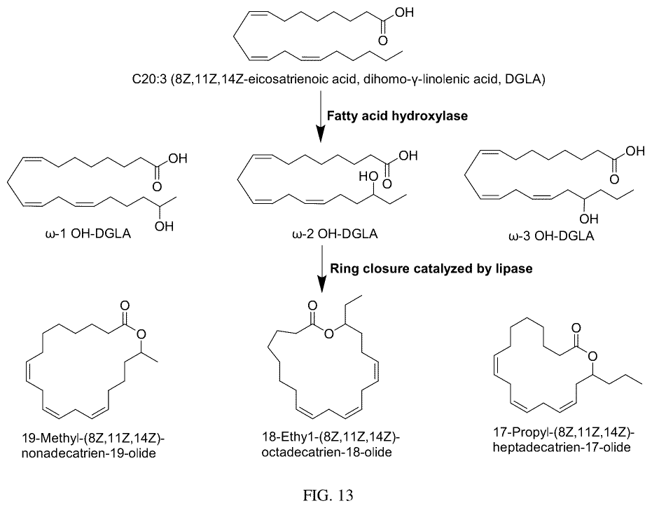

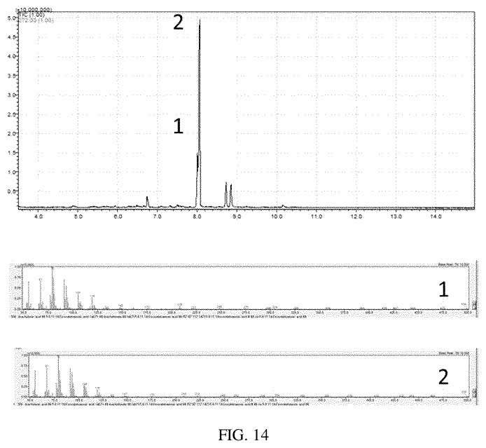

Hydroxylated DGLA was extracted by ethyl acetate and ethyl acetate phase was dried in SpeedVac™ vacuum concentrator. Then Novozym 435 in the form of acrylic resin (Sigma) was added with toluene as solvent for ring closure reaction at 60° C. with shaking. The products from hydroxy DGLA acids were analyzed by GC/MS. See FIGS. 13 and 14.

TABLE 2
Additional fatty acid substrates and lactones

| | | MuL lactone | MuL lactone | MuL lactone |
|---|---|---|---|---|
| # | Parent Fatty Acid | product 1 | product 2 | product 3 |
| 1 | 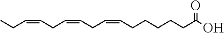 [SMILES: CC/C=C\C/C=C\C/C=C\CCCCCC(=O)O] (Roughanic acid; (7Z,10Z,13Z)- Hexadecatrienoic acid) | 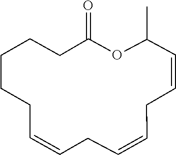 [SMILES: CC1/C=C\C/C=C\C/C=C\CCCCCC(=O)O1] | | |
| 2 | 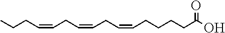 [SMILES: CCC/C=C\C/C=C\C/C=C\CCCCC(=O)O] (Hexadecatrienoic acid; (6Z,9Z,12Z)-Hexadecatrienoic acid) | 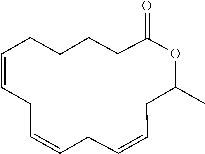 [SMILES: CC1C/C=C\C/C=C\C/C=C\CCCCC(=O)O1] | 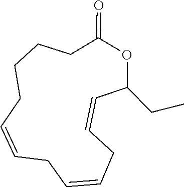 [SMILES: CCC1/C=C\C/C=C\C/C=C\CCCCC(=O)O1] | |
| 3 | 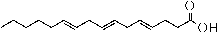 [SMILES: CCCCC/C=C/C/C=C/C/C=C/CCC(=O)O] ((4E,7E,10E)-Hexadecatrienoic acid) | 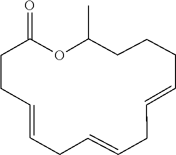 [SMILES: CC1CCC/C=C/C/C=C/C/C=C/CCC(=O)O1] | 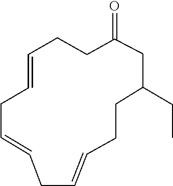 [SMILES: CCC1CC/C=C/C/C=C/C/C=C/CCC(=O)C1] | 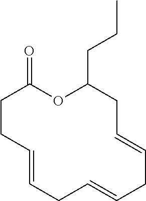 [SMILES: CCCC1C/C=C/C/C=C/C/C=C/CCC(=O)O1] |
| 4 | 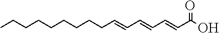 [SMILES: CCCCCCCCC/C=C/C=C/C=C/C(=O)O] ((2E,4E,6E)-Hexadecatrienoic acid) | 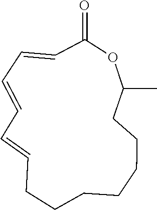 [SMILES: CC1CCCCCCC/C=C/C=C/C=C/C(=O)O1] | 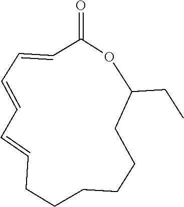 [SMILES: CCC1CCCCCC/C=C/C=C/C=C/C(=O)O1] | 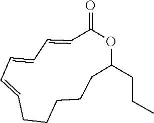 [SMILES: CCCC1CCCCC/C=C/C=C/C=C/C(=O)O1] |
| 5 | 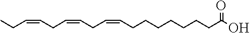 [SMILES: CC/C=C\C/C=C\C/C=C\CCCCCCCC(=O)O] (γ-linolenic acid; GLA) | 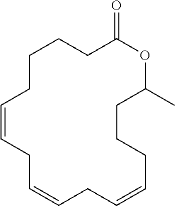 [SMILES: CC1CCC/C=C\C/C=C\C/C=C\CCCCC(=O)O1] | 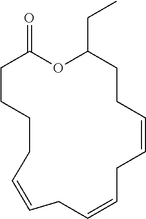 [SMILES: CCC1CC/C=C\C/C=C\C/C=C\CCCCC(=O)O1] | 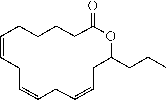 [SMILES: CCCC1C/C=C\C/C=C\C/C=C\CCCCC(=O)O1] |
| 6 | 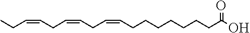 [SMILES: CC/C=C\C/C=C\C/C=C\CCCCCCCC(=O)O] (α-linolenic acid; ALA) | 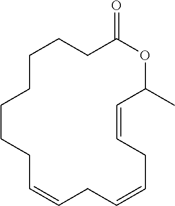 [SMILES: CC1/C=C\C/C=C\C/C=C\CCCCCCCC(=O)O1] | | |
| 7 | 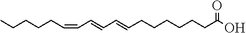 [SMILES: CCCCC\C=C/C=C/C=C/CCCCCCC(=O)O] (α-Calendic acid; (8E,10E,12Z)- Octadecatrienoic acid) | 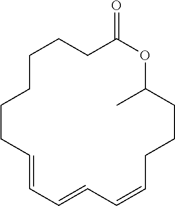 [SMILES: CC1CCC\C=C/C=C/C=C/CCCCCCC(=O)O1] | 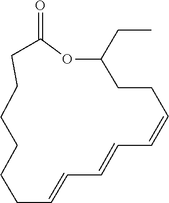 [SMILES: CCC1CC\C=C/C=C/C=C/CCCCCCC(=O)O1] | 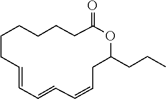 [SMILES: CCCC1C\C=C/C=C/C=C/CCCCCCC(=O)O1] |
| 8 | 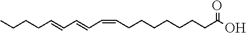 [SMILES: CCCC/C=C/C=C/C=C\CCCCCCCC(=O)O] (α-Eleostearic acid; (9Z,11E,13E)-octadeca-9,11,13- trienoic acid) | 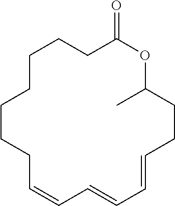 [SMILES: CC1CC/C=C/C=C/C=C\CCCCCCCC(=O)O1] | 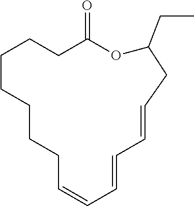 [SMILES: CCC1C/C=C/C=C/C=C\CCCCCCCC(=O)O1] | 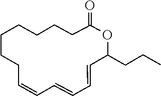 [SMILES: CCCC1/C=C/C=C/C=C\CCCCCCCC(=O)O1] |
| 9 | 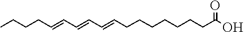 [SMILES: CCCC/C=C/C=C/C=C/CCCCCCCC(=O)O] (β-Eleostearic acid; (9E,11E,13E)-octadeca-9,11,13- trienoic acid) | 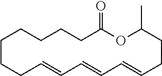 [SMILES: CC1CC/C=C/C=C/C=C/CCCCCCCC(=O)O1] | 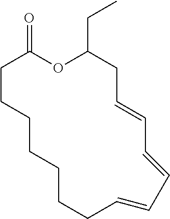 [SMILES: CCC1C/C=C/C=C/C=C/CCCCCCCC(=O)O1] | 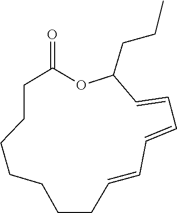 [SMILES: CCCC1/C=C/C=C/C=C/CCCCCCCC(=O)O1] |
| 10 | 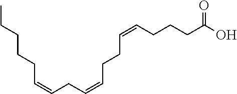 [SMILES: CCCCC/C=C\C/C=C\CC/C=C\CCCC(=O)O] (Pinolenic acid; (5Z,9Z,12Z)- octadeca-5,9,12-trienoic acid) | 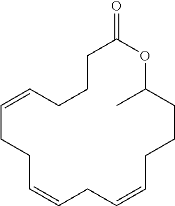 [SMILES: CC1CCC/C=C\C/C=C\CC/C=C\CCCC(=O)O1] | 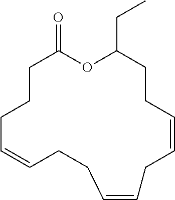 [SMILES: CCC1CC/C=C\C/C=C\CC/C=C\CCCC(=O)O1] | 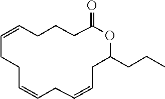 [SMILES: CCCC1C/C=C\C/C=C\CC/C=C\CCCC(=O)O1] |
| 11 | 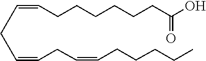 [SMILES: CCCCC/C=C\C/C=C\C/C=C\CCCCCCC(=O)O] (8Z,11Z,14Z-eicosatrienoic acid; dihomo-γ-linolenic acid; DGLA) | 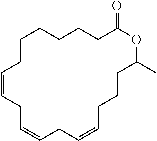 [SMILES: CC1CCC/C=C\C/C=C\C/C=C\CCCCCCC(=O)O1] | 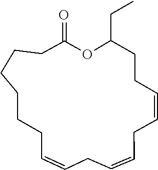 [SMILES: CCC1CC/C=C\C/C=C\C/C=C\CCCCCCC(=O)O1] | 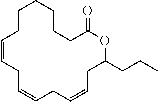 [SMILES: CCCC1C/C=C\C/C=C\C/C=C\CCCCCCC(=O)O1] |
| 12 | 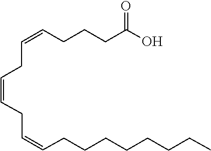 [SMILES: CCCCCCCC/C=C\C/C=C\C/C=C\CCCC(=O)O] (Mead acid; (5Z,8Z,11Z)- | 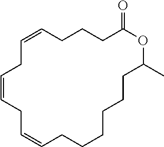 [SMILES: CC1CCCCCC/C=C\C/C=C\C/C=C\CCCC(=O)O1] | 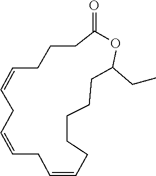 [SMILES: CCC1CCCCC/C=C\C/C=C\C/C=C\CCCC(=O)O1] | 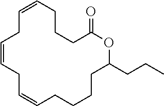 [SMILES: CCCC1CCCC/C=C\C/C=C\C/C=C\CCCC(=O)O1] |
| | Eicosa-5,8,11-trienoic acid) | | | |

### Example 4

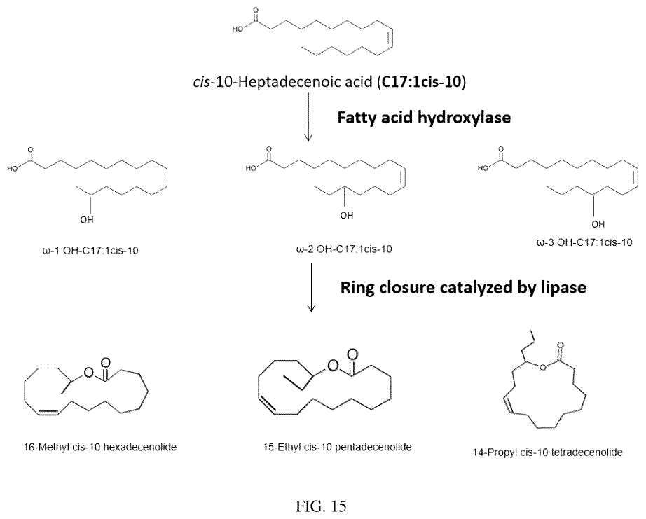

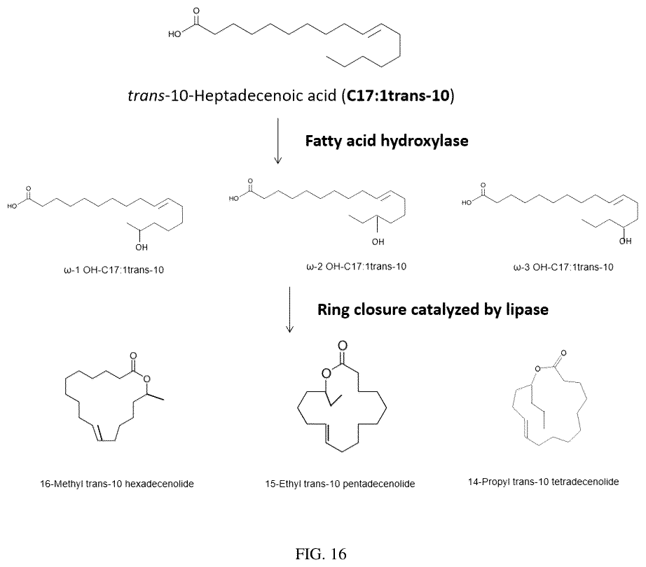

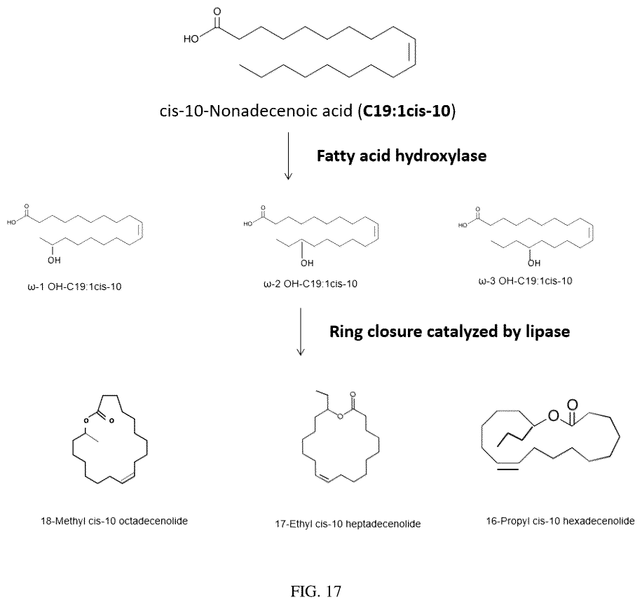

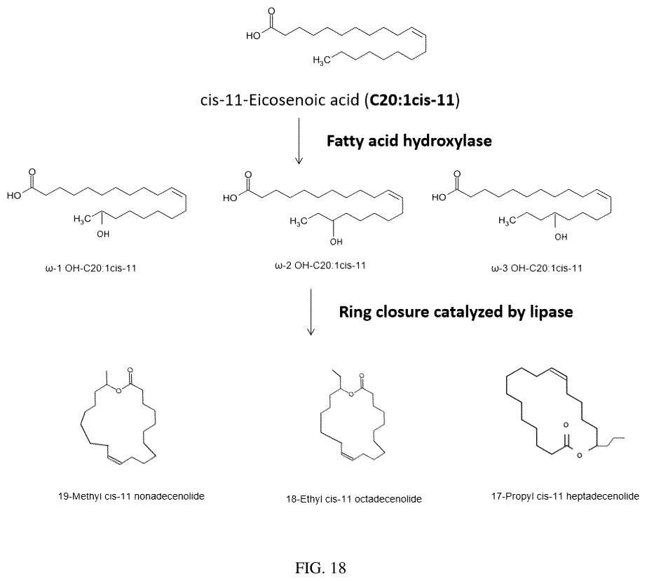

A total number of four monounsaturated fatty acids were used for musk lactone production. The biosynthetic processes are illustrated in FIGS. 15-18. The structures of the monounsaturated fatty acids and the resulting lactones are shown in Table 3.

The lactone production process used involved biosynthetic conversion of the monounsaturated fatty acids using P450 hydroxylase and lipase. The amino acid sequence of a BM3 homolog of Myceliophthora thermophile (CYP505A30) was obtained from UniProt (uniprot.org/uniprot/G2QDZ3.fasta). The corresponding gene was codon optimized for expression in Escherichia coli and synthesized by GenScript (Piscataway, NJ). The resulting gene product was then cloned into pETDuet-1 vector (AMP+, Novagen) through NdeI and XhoI sites. The resulting construct was then transformed into BL21(DE3) cells for expression.

To produce hydroxylated fatty acids, an overnight culture was used to inoculate liquid LB medium (2%) containing 100 mg/L of carbenicillin and 0.4 mM 5-aminolevulinic acid. The culture was first grown at 37° C. to an OD₆₀₀ of 0.6 and then cooled down to 16° C. Then, 1 mM IPTG was added to induce protein expression. After 16 hours of incubation at 16° C., cells were harvested by centrifugation. Harvested cell pellets were re-suspended to a concentration of 100 g/L fresh weight in 100 mM potassium phosphate buffer (pH7.0) containing 0.1% Tween 40 and 10 mM NADPH. Then, 1 g/L or 3 g/L of various fatty acids was added. The mixture was shaken at 37° C. in a shaker.

Hydroxylated fatty acids were extracted by ethyl acetate. The ethyl acetate phase was dried in a SpeedVac™ vacuum concentrator. To perform the ring closure reaction, Novozym 435 in the form of acrylic resin (Sigma) was added with toluene as a solvent and incubated at 60° C. with shaking.

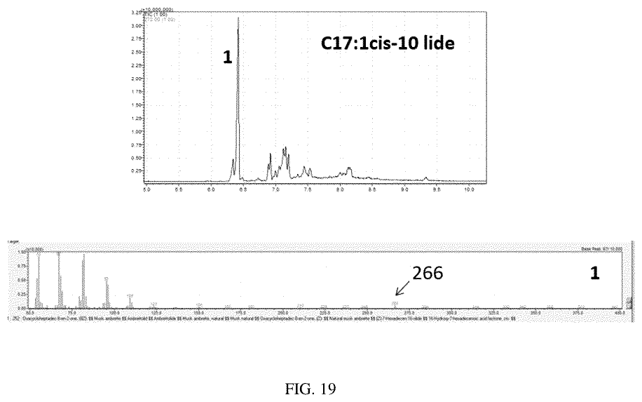

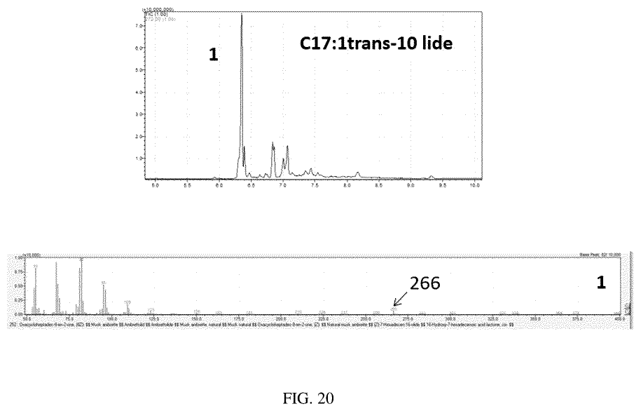

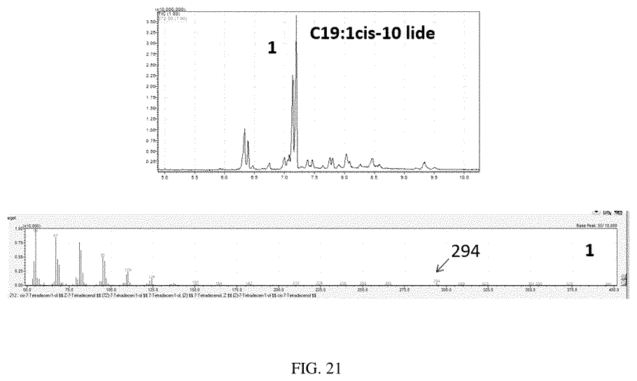

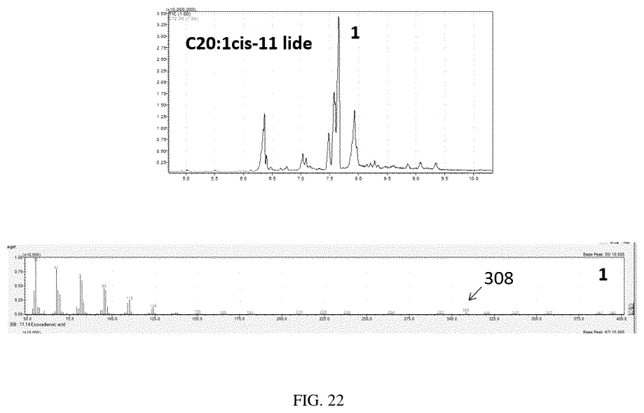

The lactone products were then analyzed by GC/MS. See FIGS. 19-22. GC/MS analysis was conducted on Shimadzu GC-2030 system coupled with GCMS-QP2020NX detector. The analytical column is SHRXI-5MS (thickness 0.25 μm; length 30 m; diameter 0.2 mm) and the injection temperature was 265° C. under split mode. The temperature gradient was 0-3 min 150° C.; 3-6.7 min 150° C. to 260° C., a gradient of 30; 6.7-15.7 min, 260° C.

TABLE 3

| | | MuL Lactone | MuL Lactone | MuL Lactone |
|---|---|---|---|---|
| # | Parent Fatty Acid | Product 1 | Product 2 | Product 3 |
| 1 | 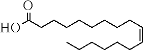 [SMILES: CCCCCC/C=C\CCCCCCCCC(=O)O] cis-10-Heptadecenoic acid (C17:1cis-10) | 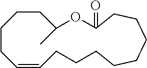 [SMILES: CC1CCCC/C=C\CCCCCCCCC(=O)O1] | 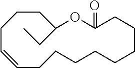 [SMILES: CCC1CCC/C=C\CCCCCCCCC(=O)O1] | 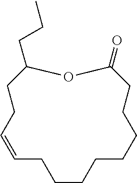 [SMILES: CCCC1CC/C=C\CCCCCCCCC(=O)O1] |
| 2 | 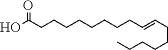 [SMILES: CCCCCC/C=C/CCCCCCCCC(=O)O] trans-10-Heptadecenoic acid (C17:1trans-10) | 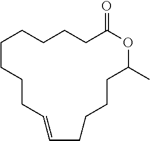 [SMILES: CC1CCCC/C=C/CCCCCCCCC(=O)O1] | 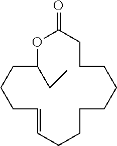 [SMILES: CCC1CCC/C=C/CCCCCCCCC(=O)O1] | 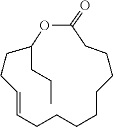 [SMILES: CCCC1CC/C=C/CCCCCCCCC(=O)O1] |
| 3 | 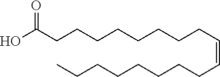 [SMILES: CCCCCCCC/C=C\CCCCCCCCC(=O)O] cis-10-Nonadecenoic acid (C19:1cis-10) | 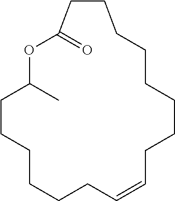 [SMILES: CC1CCCCCC/C=C\CCCCCCCCC(=O)O1] | 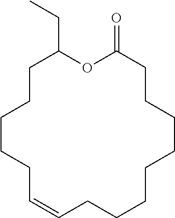 [SMILES: CCC1CCCCC/C=C\CCCCCCCCC(=O)O1] | 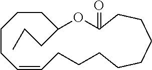 [SMILES: CCCC1CCCC/C=C\CCCCCCCCC(=O)O1] |
| 4 | 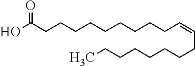 [SMILES: CCCCCCCC/C=C\CCCCCCCCCC(=O)O] cis-11-Eicosenoic acid (C20:1cis-11) | 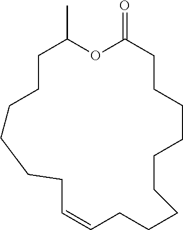 [SMILES: CC1CCCCCC/C=C\CCCCCCCCCC(=O)O1] | 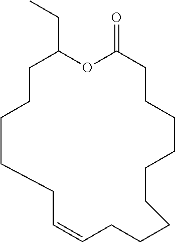 [SMILES: CCC1CCCCC/C=C\CCCCCCCCCC(=O)O1] | 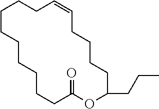 [SMILES: CCCC1CCCC/C=C\CCCCCCCCCC(=O)O1] |

### REFERENCES

Baker G J, Girvan H M, Matthews S, McLean K J, Golovanova M, Waltham T N, Rigby S E J, Nelson D R, Blankley R T, Munro A W. (2017) Expression, Purification, and Biochemical Characterization of the Flavocytochrome P450 CYP505A30 from Myceliophthora thermophile. ACS Omega 2: 4705-4724.
Miura Y, Fulco A J. (1975) Omega-1, Omega-2 and Omega-3 hydroxylation of long-chain fatty acids, amides and alcohols by a soluble enzyme system from Bacillus megaterium. Biochim. Biophys. Acta. 388: 305-317.
Wen L P, Fulco A J. (1987) Cloning of the gene encoding a catalytically self-sufficient cytochrome P-450 fatty acid monooxygenase induced by barbiturates in Bacillus megaterium and its functional expression and regulation in heterologous (Escherichia coli) and homologous (Bacillus megaterium) hosts. J. Biol. Chem. 262: 6676-6682.

### Amino Acid and Nucleotide Sequences

Amino acid sequence of cytochrome P450 CYP102A1 from Bacillus megaterium
SEQ ID NO: 1
MTIKEMPQPKTFGELKNLPLLNTDKPVQALMKIADELGEIFKFEAPGRVTRYLSSQRLI
KEACDESRFDKNLSQALKFVRDFAGDGLFTSWTHEKNWKKAHNILLPSFSQQAMKG
YHAMMVDIAVQLVQKWERLNADEHIEVPEDMTRLTLDTIGLCGFNYRFNSFYRDQPH
PFITSMVRALDEAMNKLQRANPDDPAYDENKRQFQEDIKVMNDLVDKIIADRKASGE
QSDDLLTHMLNGKDPETGEPLDDENIRYQIITFLIAGHETTSGLLSFALYFLVKNPHVL
QKAAEEAARVLVDPVPSYKQVKQLKYVGMVLNEALRLWPTAPAFSLYAKEDTVLGG
EYPLEKGDELMVLIPQLHRDKTIWGDDVEEFRPERFENPSAIPQHAFKPFGNGQRACIG
QQFALHEATLVLGMMLKHFDFEDHTNYELDIKETLTLKPEGFVVKAKSKKIPLGGIPS
PSTEQSAKKVRKKAENAHNTPLLVLYGSNMGTAEGTARDLADIAMSKGFAPQVATL
DSHAGNLPREGAVLIVTASYNGHPPDNAKQFVDWLDQASADEVKGVRYSVFGCGDK
NWATTYQKVPAFIDETLAAKGAENIADRGEADASDDFEGTYEEWREHMWSDVAAYF
NLDIENSEDNKSTLSLQFVDSAADMPLAKMHGAFSTNVVASKELQQPGSARSTRHLEI
ELPKEASYQEGDHLGVIPRNYEGIVNRVTARFGLDASQQIRLEAEEEKLAHLPLAKTVS
VEELLQYVELQDPVTRTQLRAMAAKTVCPPHKVELEALLEKQAYKEQVLAKRLTML
ELLEKYPACEMKFSEFIALLPSIRPRYYSISSSPRVDEKQASITVSVVSGEAWSGYGEYK
GIASNYLAELQEGDTITCFISTPQSEFTLPKDPETPLIMVGPGTGVAPFRGFVQARKQLK
EQGQSLGEAHLYFGCRSPHEDYLYQEELENAQSEGIITLHTAFSRMPNQPKTYVQHVM
EQDGKKLIELLDQGAHFYICGDGSQMAPAVEATLMKSYADVHQVSEADARLWLQQL
EEKGRYAKDVWAG
Nucleotide sequence of cytochrome P450 CYP102A1 from Bacillus megaterium
SEQ ID NO: 2
ATGACAATTAAAGAAATGCCTCAGCCAAAAACGTTTGGAGAGCTTAAAAATTTAC
CGTTATTAAACACAGATAAACCGGTTCAAGCTTTGATGAAAATTGCGGATGAATT
AGGAGAAATCTTTAAATTCGAGGCGCCTGGTCGTGTAACGCGCTACTTATCAAGTC
AGCGTCTAATTAAAGAAGCATGCGATGAATCACGCTTTGATAAAAACTTAAGTCA
AGCGCTTAAATTTGTACGTGATTTTGCAGGAGACGGGTTATTTACAAGCTGGACGC
ATGAAAAAAATTGGAAAAAAGCGCATAATATCTTACTTCCAAGCTTCAGTCAGCA
GGCAATGAAAGGCTATCATGCGATGATGGTCGATATCGCCGTGCAGCTTGTTCAA
AAGTGGGAGCGTCTAAATGCAGATGAGCATATTGAAGTACCGGAAGACATGACAC
GTTTAACGCTTGATACAATTGGTCTTTGCGGCTTTAACTATCGCTTTAACAGCTTTT
ACCGAGATCAGCCTCATCCATTTATTACAAGTATGGTCCGTGCACTGGATGAAGCA
ATGAACAAGCTGCAGCGAGCAAATCCAGACGACCCAGCTTATGATGAAAACAAGC
GCCAGTTTCAAGAAGATATCAAGGTGATGAACGACCTAGTAGATAAAATTATTGC
AGATCGCAAAGCAAGCGGTGAACAAAGCGATGATTTATTAACGCATATGCTAAAC
GGAAAAGATCCAGAAACGGGTGAGCCGCTTGATGACGAGAACATTCGCTATCAAA
TTATTACATTCTTAATTGCGGGACACGAAACAACAAGTGGTCTTTTATCATTTGCG
CTGTATTTCTTAGTGAAAAATCCACATGTATTACAAAAAGCAGCAGAAGAAGCAG
CACGAGTTCTAGTAGATCCTGTTCCAAGCTACAAACAAGTCAAACAGCTTAAATAT
GTCGGCATGGTCTTAAACGAAGCGCTGCGCTTATGGCCAACTGCTCCTGCGTTTTC
CCTATATGCAAAAGAAGATACGGTGCTTGGAGGAGAATATCCTTTAGAAAAAGGC
GACGAACTAATGGTTCTGATTCCTCAGCTTCACCGTGATAAAACAATTTGGGGAGA
CGATGTGGAAGAGTTCCGTCCAGAGCGTTTTGAAAATCCAAGTGCGATTCCGCAG
CATGCGTTTAAACCGTTTGGAAACGGTCAGCGTGCGTGTATCGGTCAGCAGTTCGC
TCTTCATGAAGCAACGCTGGTACTTGGTATGATGCTAAAACACTTTGACTTTGAAG
ATCATACAAACTACGAGCTGGATATTAAAGAAACTTTAACGTTAAAACCTGAAGG
CTTTGTGGTAAAAGCAAAATCGAAAAAAATTCCGCTTGGCGGTATTCCTTCACCTA
GCACTGAACAGTCTGCTAAAAAAGTACGCAAAAAGGCAGAAAACGCTCATAATAC
GCCGCTGCTTGTGCTATACGGTTCAAATATGGGAACAGCTGAAGGAACGGCGCGT
GATTTAGCAGATATTGCAATGAGCAAAGGATTTGCACCGCAGGTCGCAACGCTTG
ATTCACACGCCGGAAATCTTCCGCGCGAAGGAGCTGTATTAATTGTAACGGCGTCT
TATAACGGTCATCCGCCTGATAACGCAAAGCAATTTGTCGACTGGTTAGACCAAG
CGTCTGCTGATGAAGTAAAAGGCGTTCGCTACTCCGTATTTGGATGCGGCGATAAA
AACTGGGCTACTACGTATCAAAAAGTGCCTGCTTTTATCGATGAAACGCTTGCCGC
TAAAGGGGCAGAAAACATCGCTGACCGCGGTGAAGCAGATGCAAGCGACGACTTT
GAAGGCACATATGAAGAATGGCGTGAACATATGTGGAGTGACGTAGCAGCCTACT
TTAACCTCGACATTGAAAACAGTGAAGATAATAAATCTACTCTTTCACTTCAATTT
GTCGACAGCGCCGCGGATATGCCGCTTGCGAAAATGCACGGTGCGTTTTCAACGA
ACGTCGTAGCAAGCAAAGAACTTCAACAGCCAGGCAGTGCACGAAGCACGCGAC
ATCTTGAAATTGAACTTCCAAAAGAAGCTTCTTATCAAGAAGGAGATCATTTAGGT
GTTATTCCTCGCAACTATGAAGGAATAGTAAACCGTGTAACAGCAAGGTTCGGCC
TAGATGCATCACAGCAAATCCGTCTGGAAGCAGAAGAAGAAAAATTAGCTCATTT
GCCACTCGCTAAAACAGTATCCGTAGAAGAGCTTCTGCAATACGTGGAGCTTCAA
GATCCTGTTACGCGCACGCAGCTTCGCGCAATGGCTGCTAAAACGGTCTGCCCGCC
GCATAAAGTAGAGCTTGAAGCCTTGCTTGAAAAGCAAGCCTACAAAGAACAAGTG
CTGGCAAAACGTTTAACAATGCTTGAACTGCTTGAAAAATACCCGGCGTGTGAAA
TGAAATTCAGCGAATTTATCGCCCTTCTGCCAAGCATACGCCCGCGCTATTACTCG
ATTTCTTCATCACCTCGTGTCGATGAAAAACAAGCAAGCATCACGGTCAGCGTTGT
CTCAGGAGAAGCGTGGAGCGGATATGGAGAATATAAAGGAATTGCGTCGAACTAT
CTTGCCGAGCTGCAAGAAGGAGATACGATTACGTGCTTTATTTCCACACCGCAGTC
AGAATTTACGCTGCCAAAAGACCCTGAAACGCCGCTTATCATGGTCGGACCGGGA
ACAGGCGTCGCGCCGTTTAGAGGCTTTGTGCAGGCGCGCAAACAGCTAAAAGAAC
AAGGACAGTCACTTGGAGAAGCACATTTATACTTCGGCTGCCGTTCACCTCATGAA
GACTATCTGTATCAAGAAGAGCTTGAAAACGCCCAAAGCGAAGGCATCATTACGC
TTCATACCGCTTTTTCTCGCATGCCAAATCAGCCGAAAACATACGTTCAGCACGTA
ATGGAACAAGACGGCAAGAAATTGATTGAACTTCTTGATCAAGGAGCGCACTTCT
ATATTTGCGGAGACGGAAGCCAAATGGCACCTGCCGTTGAAGCAACGCTTATGAA
AAGCTATGCTGACGTTCACCAAGTGAGTGAAGCAGACGCTCGCTTATGGCTGCAG
CAGCTAGAAGAAAAAGGCCGATACGCAAAAGACGTGTGGGCTGGGTAA
Amino acid sequence of cytochrome P450 CYP505A30 from Myceliophthora
thermophile
SEQ ID NO: 3
MADKTTETVPIPGPPGLPLVGNALAFDSELPLRTFQEFAEEYGEIYRLTLPTGTTLVVSS
QALVHELCDDKRFKKPVAAALAEVRNGVNDGLFTAREEEPNWGIAHRILMPAFGPAS
IQGMFTEMHEIASQLALKWARHGPDTPIFVTDDFTRLTLDTLALCTMNFRENSYYHDE
LHPFINAMGNFLTESGARAMRPAITSIFHQAANRKYWEDIEVLRKTAQGVLDTRRKHP
TNRKDLLSAMLDGVDAKTGQKLSDSSIIDNLITFLIAGHETTSGLLSFAFYLLIKHQDA
YRKAQEEVDRVIGKGPIKVEHIKKLPYIAAVLRETLRLCPTIPIINRAAKQDEVIGGKYA
VAKDQRLALLLAQSHLDPAVYGETAKQFIPERMLDENFERLNREYPDCWKPFGTGMR
ACIGRPFAWQEAVLVMAMLLQNFDFVLHDPYYELHYKQTLTTKPKDFYMRAILRDG
LTATELEHRLAGNAASVARSGGGGGGPSKPTAQKTSPAEAKPMSIFYGSNTGTCESLA
QRLATDAASHGYAAAAVEPLDTATEKLPTDRPVVIITASFEGQPPDNAAKFCGWLKN
LEGDELKNVSYAVFGCGHHDWSQTFHRIPKLVHQTMKAHGASPICDEGLTDVAEGN
MFTDFEQWEDDVFWPAVRARYGAAGAVAETEDAPGSDGLNIHFSSPRSSTLRQDVRE
ATVVGEALLTAPDAPPKKHIEVQLPDGATYKVGDYLAVLPVNSKESIGRVMRKFQLS
WDSHVTIASDRWTALPTGTPVPAYDVLGSYVELSQPATKRGILRLADAAEDEATKAE
LQKLAGDLYTSEISLKRASVLDLLDRFPSISLPFGTFLSLLPPIRPRQYSISSSPLNDPSRA
TLTYSLLDSPSLANPSRRFVGVATSYLSSLVRGDKLLVSVRPTHTAFRLPDEDKMGET
AIICVGAGSGLAPFRGFIQERAALLAKGTQLAAALLFYGCRSPEKDDLYRDEFDKWQE
SGAVDVRRAFSRVDSDDTEARGCRHVQDRLWHDREEVKALWDRGARVYVCGSRQV
GEGVKTAMGRIVLGEEDAEDAISKWYETVRNDRYATDVFD
Codon optimized nucleotide sequence of cytochrome P450 CYP505A30 from
Myceliophthora thermophile
SEQ ID NO: 4
ATGGCGGATAAGACCACCGAAACCGTGCCGATTCCGGGTCCGCCGGGCCTGCCGC
TGGTTGGTAATGCGCTGGCGTTTGATAGCGAACTGCCGCTGCGTACCTTCCAGGAA
TTTGCGGAGGAATACGGCGAGATCTATCGTCTGACCCTGCCGACCGGTACCACCCT
GGTGGTTAGCAGCCAAGCGCTGGTTCACGAACTGTGCGACGATAAGCGTTTCAAG
AAGCCGGTTGCTGCGGCGCTGGCGGAAGTGCGTAACGGCGTTAACGACGGTCTGT
TTACCGCGCGTGAAGAGGAGCCGAACTGGGGCATCGCGCACCGTATTCTGATGCC
GGCGTTTGGTCCGGCGAGCATTCAGGGCATGTTTACCGAAATGCACGAGATCGCG
AGCCAACTGGCGCTGAAATGGGCGCGTCACGGTCCGGACACCCCGATTTTCGTTA
CCGACGATTTTACCCGTCTGACCCTGGATACCCTGGCGCTGTGCACCATGAACTTC
CGTTTTAACAGCTACTATCACGACGAACTGCACCCGTTCATCAACGCGATGGGCAA
CTTTCTGACCGAGAGCGGTGCGCGTGCGATGCGTCCGGCGATCACCAGCATTTTCC
ACCAGGCGGCGAACCGTAAGTACTGGGAAGATATTGAGGTTCTGCGTAAAACCGC
GCAAGGTGTGCTGGACACCCGTCGTAAGCACCCGACCAACCGTAAAGATCTGCTG
AGCGCGATGCTGGACGGCGTGGATGCGAAAACCGGTCAGAAACTGAGCGACAGC
AGCATCATTGATAACCTGATCACCTTTCTGATTGCGGGCCACGAAACCACCAGCGG
TCTGCTGAGCTTCGCGTTTTACCTGCTGATTAAGCACCAGGACGCGTATCGTAAAG
CGCAAGAAGAGGTGGATCGTGTTATCGGCAAGGGCCCGATTAAAGTTGAACACAT
CAAGAAACTGCCGTACATCGCGGCGGTGCTGCGTGAAACCCTGCGTCTGTGCCCG
ACCATTCCGATCATTAACCGTGCGGCGAAGCAGGACGAAGTTATCGGTGGCAAGT
ACGCGGTGGCGAAAGATCAGCGTCTGGCGCTGCTGCTGGCGCAAAGCCACCTGGA
CCCGGCGGTTTATGGCGAAACCGCGAAGCAATTCATTCCGGAGCGTATGCTGGAC
GAAAACTTTGAGCGTCTGAACCGTGAGTATCCGGATTGCTGGAAACCGTTCGGTA
CCGGCATGCGTGCGTGCATCGGTCGTCCGTTTGCGTGGCAGGAAGCGGTGCTGGTT
ATGGCGATGCTGCTGCAAAACTTCGACTTTGTTCTGCACGATCCGTACTATGAGCT
GCACTACAAGCAGACCCTGACCACCAAGCCGAAAGACTTCTATATGCGTGCGATC
CTGCGTGATGGCCTGACCGCGACCGAACTGGAGCACCGTCTGGCGGGTAACGCGG
CGAGCGTGGCGCGTAGCGGTGGCGGTGGCGGTGGCCCGAGCAAACCGACCGCGC
AGAAAACCAGCCCGGCGGAAGCGAAACCGATGAGCATCTTCTACGGCAGCAACA
CCGGTACCTGCGAGAGCCTGGCGCAACGTCTGGCGACCGATGCGGCGAGCCACGG
TTATGCTGCGGCGGCGGTGGAACCGCTGGACACCGCGACCGAGAAGCTGCCGACC
GATCGTCCGGTGGTTATCATTACCGCGAGCTTCGAGGGTCAGCCGCCGGACAACG
CGGCGAAGTTTTGCGGCTGGCTGAAAAACCTGGAAGGTGATGAGCTGAAAAACGT
GAGCTACGCGGTTTTCGGTTGCGGCCACCACGACTGGAGCCAGACCTTTCACCGTA
TTCCGAAGCTGGTTCACCAAACCATGAAAGCGCACGGTGCGAGCCCGATCTGCGA
CGAAGGCCTGACCGATGTGGCGGAGGGTAACATGTTCACCGATTTTGAACAATGG
GAGGACGATGTGTTCTGGCCGGCGGTTCGTGCGCGTTATGGCGCGGCGGGTGCGG
TTGCGGAAACCGAGGACGCGCCGGGTAGCGATGGTCTGAACATCCACTTTAGCAG
CCCGCGTAGCAGCACCCTGCGTCAGGACGTGCGTGAAGCGACCGTGGTTGGTGAA
GCGCTGCTGACCGCGCCGGATGCGCCGCCGAAGAAACACATTGAAGTTCAACTGC
CGGACGGCGCGACCTACAAAGTGGGTGATTATCTGGCGGTGCTGCCGGTTAACAG
CAAGGAGAGCATTGGTCGTGTTATGCGTAAATTCCAGCTGAGCTGGGACAGCCAC
GTGACCATCGCGAGCGATCGTTGGACCGCGCTGCCGACCGGTACCCCGGTGCCGG
CGTACGACGTTCTGGGTAGCTATGTGGAGCTGAGCCAACCGGCGACCAAACGTGG
TATCCTGCGTCTGGCGGATGCGGCGGAAGATGAGGCGACCAAGGCGGAACTGCAA
AAACTGGCGGGTGATCTGTACACCAGCGAGATTAGCCTGAAACGTGCGAGCGTTC
TGGACCTGCTGGATCGTTTCCCGAGCATCAGCCTGCCGTTCGGTACCTTTCTGAGC
CTGCTGCCGCCGATTCGTCCGCGTCAATACAGCATCAGCAGCAGCCCGCTGAACG
ACCCGAGCCGTGCGACCCTGACCTATAGCCTGCTGGATAGCCCGAGCCTGGCGAA
CCCGAGCCGTCGTTTCGTGGGCGTTGCGACCAGCTACCTGAGCAGCCTGGTTCGTG
GTGACAAGCTGCTGGTGAGCGTTCGTCCGACCCACACCGCGTTTCGTCTGCCGGAC
GAAGATAAAATGGGTGAAACCGCGATCATTTGCGTGGGTGCGGGTAGCGGTCTGG
CGCCGTTCCGTGGTTTTATCCAGGAACGTGCGGCGCTGCTGGCGAAAGGTACCCA
ACTGGCGGCGGCGCTGCTGTTCTACGGTTGCCGTAGCCCGGAGAAGGACGATCTG
TATCGTGACGAATTCGATAAATGGCAAGAGAGCGGTGCGGTGGATGTTCGTCGTG
CGTTTAGCCGTGTTGATAGCGACGATACCGAGGCGCGTGGTTGCCGTCACGTTCAG
GACCGTCTGTGGCACGATCGTGAAGAGGTGAAGGCGCTGTGGGACCGTGGCGCGC
GTGTGTACGTTTGCGGTAGCCGTCAAGTGGGCGAAGGTGTTAAAACCGCGATGGG
CCGTATCGTGCTGGGTGAAGAGGACGCGGAGGATGCGATCAGCAAGTGGTATGAA
ACCGTGCGTAATGACCGTTATGCGACCGATGTGTTCGACTAA
Amino acid sequence of Lipase B from Candida antarctica
SEQ ID NO: 5
MKLLSLTGVAGVLATCVAATPLVKRLPSGSDPAFSQPKSVLDAGLTCQGASPSSVSKP
ILLVPGTGTTGPQSFDSNWIPLSTQLGYTPCWISPPPFMLNDTQVNTEYMVNAITALYA
GSGNNKLPVLTWSQGGLVAQWGLTFFPSIRSKVDRLMAFAPDYKGTVLAGPLDALA
VSAPSVWQQTTGSALTTALRNAGGLTQIVPTTNLYSATDEIVQPQVSNSPLDSSYLENG
KNVQAQAVCGPLFVIDHAGSLTSQFSYVVGRSALRSTTGQARSADYGITDCNPLPAND
LTPEQKVAAAALLAPAAAAIVAGPKQNCEPDLMPYARPFAVGKRTCSGIVTP

1. A method of producing a lactone, the method comprising:
(i) preparing a first reaction mixture comprising one or more fatty acids, a cytochrome P450 hydroxylase, and NADPH;
(ii) incubating the first reaction mixture of for a sufficient time to produce hydroxyl fatty acids selected from ω-1 hydroxyl fatty acids, ω-2 hydroxyl fatty acid, ω-3 hydroxyl fatty acid, and combinations thereof;
(iii) preparing a second reaction mixture comprising the hydroxyl fatty acids produced in step (ii) and a lipase; and
(iv) incubating the second reaction mixture for a sufficient time to produce the lactone.

2. The method of claim 1, wherein step (ii) further comprises isolating the hydroxyl fatty acids from the first reaction mixture.

3. The method of claim 1, wherein the cytochrome P450 hydroxylase comprises:
an amino acid sequence that is at least 80% identical to the amino acid sequence of SEQ ID NO: 1 or SEQ ID NO: 3; or
the amino acid sequence of SEQ ID NO: 1 or SEQ ID NO: 3.

4. (canceled)

5. The method of claim 1, wherein the lipase:
is lipase B from Candida antarctica;
comprises an amino acid sequence that is at least 80% identical to the amino acid sequence of SEQ ID NO: 5; and/or
comprises the amino acid sequence of SEQ ID NO: 5.

6.-7. (canceled)

8. The method of claim 1, wherein the lipase is immobilized on a solid support.

9. The method of claim 1, wherein the second reaction mixture further comprises a solvent, optionally wherein the solvent is toluene or dichloroethane.

10. The method of claim 1, wherein:
the hydroxyl fatty acids in the second reaction mixture are at a total concentration of 0.02-0.1 M, optionally wherein the hydroxyl fatty acids are at a total concentration of 0.025-0.5 M; and/or
the lipase in the second reaction mixture is at a concentration of 20-150 g/L, optionally wherein the lipase is at a concentration of 50-100 g/L.

11. (canceled)

12. The method of claim 1, wherein step (iv) further comprises isolating the lactone.

13. The method of claim 1, wherein the one or more fatty acids of step (i) comprise a linear fatty acid comprising 12-28 carbon atoms, optionally wherein the one or more fatty acids of step (i) comprise a linear fatty acid comprising 15, 16, 17, 18, or 20 carbon atoms.

14. The method of claim 1, wherein the one or more fatty acids of step (i) comprise a saturated fatty acid.

15. The method of claim 1, wherein the one or more fatty acids of step (i) comprise an unsaturated fatty acid, optionally wherein the unsaturated fatty acid comprises at least one double bond, optionally wherein the unsaturated fatty acid comprises at least one Z double bond.

16. The method of claim 1, wherein the one or more fatty acids of step (i) are selected from the group consisting of: 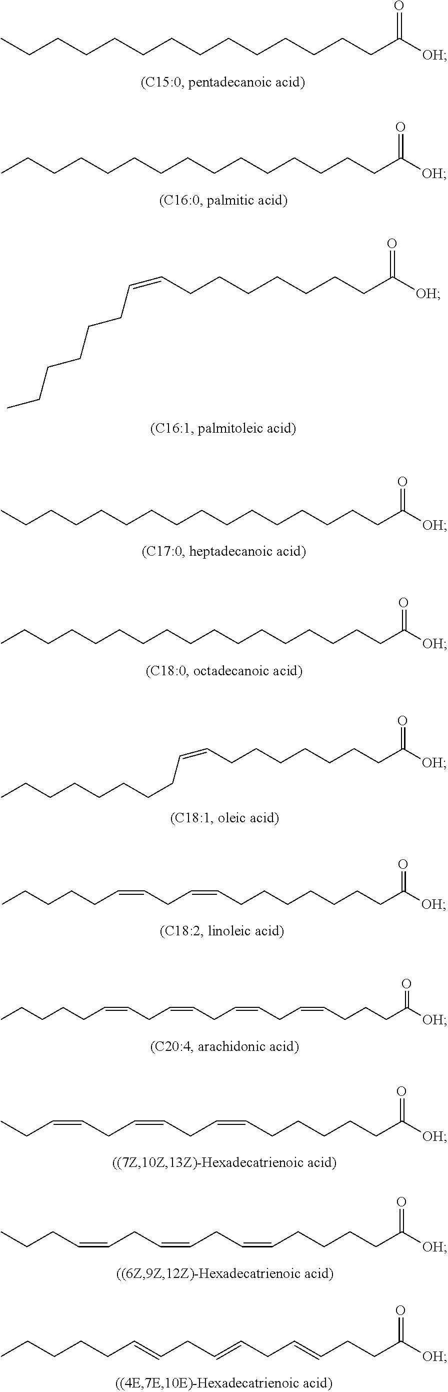 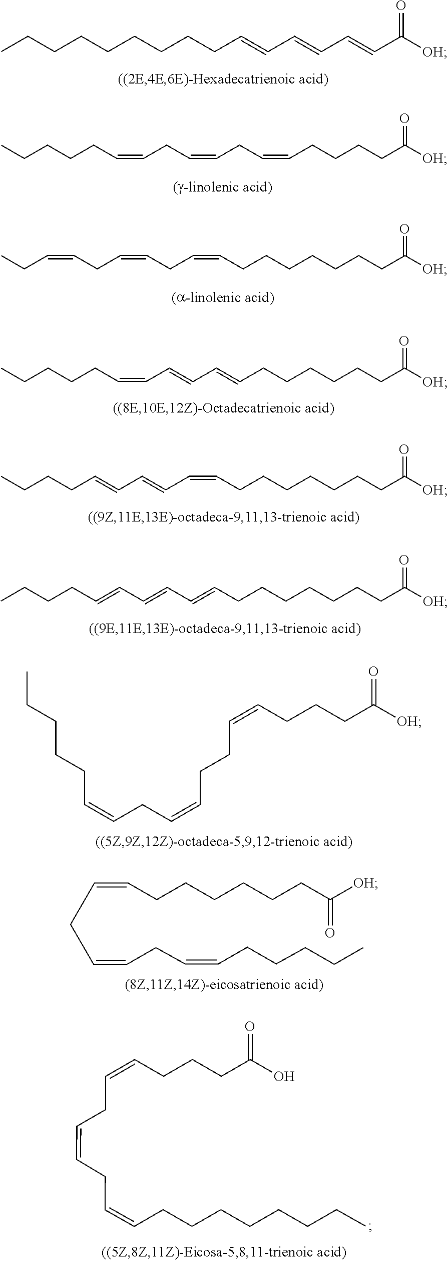
and combinations thereof.

17. The method of claim 1, wherein the one or more fatty acids of step (i) are selected from the group consisting of:

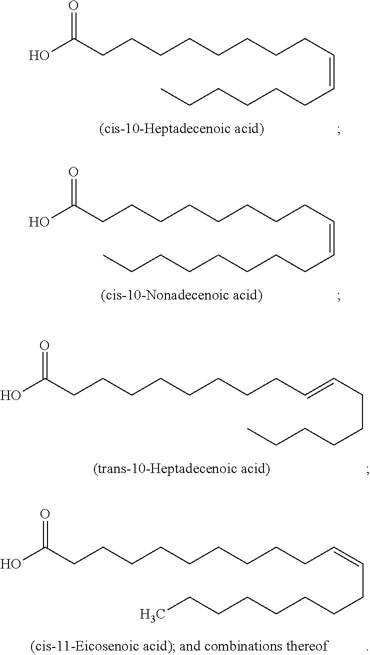 [SMILES: CCCCCC/C=C\CCCCCCCCC(=O)O ; CCCCCCCC/C=C\CCCCCCCCC(=O)O ; CCCCCC/C=C/CCCCCCCCC(=O)O ; CCCCCCCC/C=C\CCCCCCCCCC(=O)O]

18. The method of claim 1, wherein the lactone comprises one or more compounds of the formula: 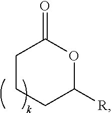
wherein:
R is methyl, ethyl, or n-propyl;
each [symbol] is independently a single bond, E double bond, Z double bond, or triple bond, as valency permits; and
k is an integer between 6 and 30, inclusive.

19. The method of claim 1, wherein the lactone comprises one or more compounds of the formula: 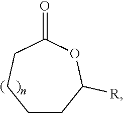
wherein:
R is methyl, ethyl, or n-propyl;
each [symbol] is independently a single bond, E double bond, Z double bond, or triple bond, as valency permits; and
n is an integer between 6 and 20, inclusive.

20.-43. (canceled)

44. The method of claim 1, wherein the first reaction mixture is in vitro; optionally wherein the first reaction is a cell-based reaction mixture; further optionally wherein the cell-based reaction mixture comprises a cell selected from the group consisting of a yeast, a plant, an alga, a fungus, and a bacterium.

45.-48. (canceled)

49. The method of claim 1, wherein the lactone produced in step (iv);
has a purity of at least 70%; and/or
has musk notes.

50. (canceled)

51. A lactone of the formula: 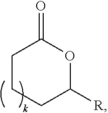
wherein:
R is methyl, ethyl, or n-propyl;
each [symbol] is independently a single bond, E double bond, Z double bond, or triple bond, as valency permits; and
k is an integer between 6 and 30, inclusive;
provided that the lactone is not of the formula:

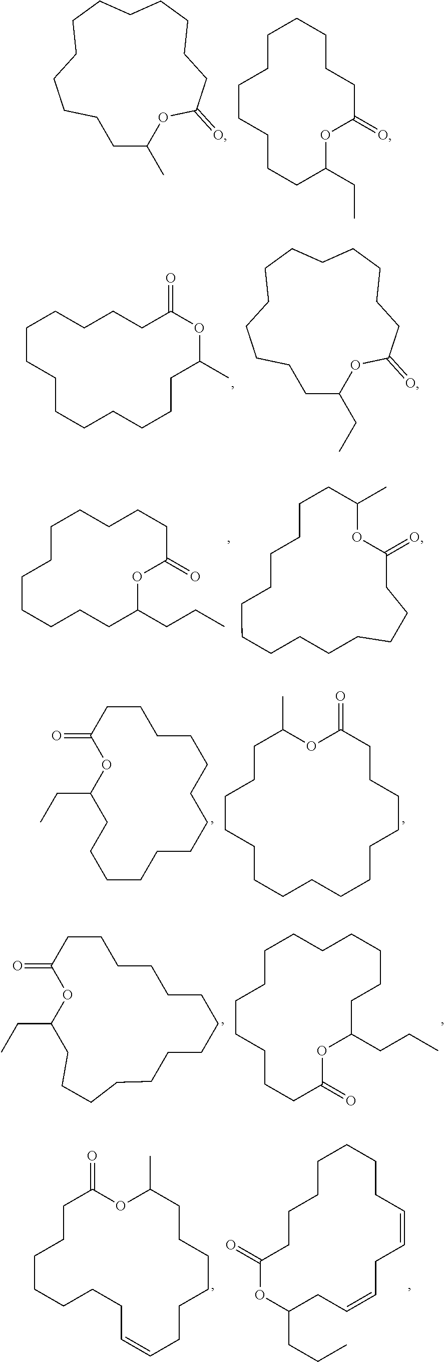

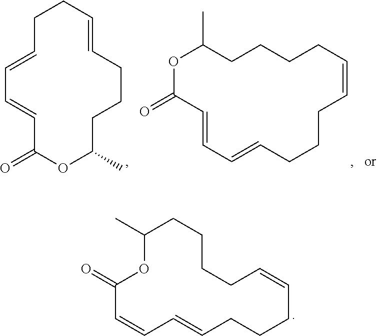

52. The lactone of claim 51, wherein the lactone is of the formula: 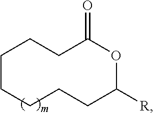
wherein:
R is methyl, ethyl, or n-propyl;
each [symbol] is independently a single bond or Z double bond, as valency permits, wherein 0, 1, 2, or 4 [symbol] are Z double bonds; and
m is an integer between 4 and 11, inclusive;
provided that the lactone is not of the formula:

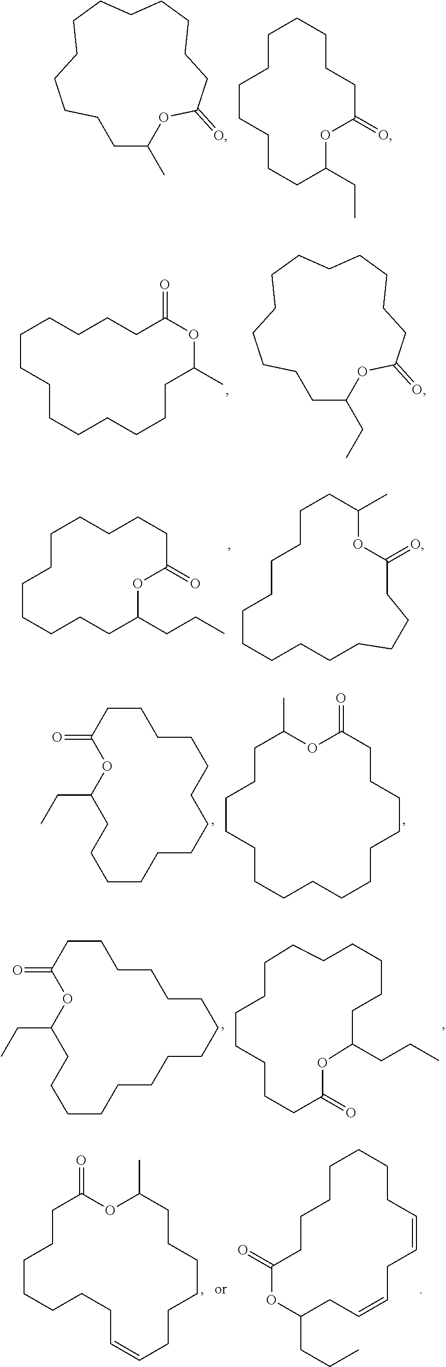

53. The method of claim 18, wherein;
each [symbol] is a single bond;
one [symbol] is an E or Z double bond, and the remining, [symbol] are single bonds;
two [symbol] are independently E or Z double bonds, and the remining, [symbol] are single bonds;
three [symbol] are independently E or Z double bonds, and the remining, [symbol] are single bonds;
four [symbol] are independently E or Z double bonds, and the remining, [symbol] are single bonds;
each double bond if present is a Z double bond;
each double bond if present is an E double bond;
the lactone does not comprise any one of C═C═C, C═C≡C, and C≡C═C;
k is 8, 9, 10, 11, 12, 13, or 15;
the chiral carbon atom is of the S configuration; and/or
the chiral carbon atom is of the R configuration.

54.-70. (canceled)
